(19) United States
(12) Patent Application Publication
(10) Pub. No.: US 2022/0213153 A1
(43) Pub. Date: Jul. 7, 2022

(54) WORMS Scaffolds: Multi-scale protein complexes

(71) Applicants: University of Washington, Seattle, WA (US); NEW YORK UNIVERSITY, New York, NY (US)

(72) Inventors: Yang HSIA, Seattle, WA (US); Rubul MOUT, Seattle, WA (US); Natasha EDMAN, Seattle, WA (US); Ivan VULOVIC, Seattle, WA (US); Una NATTERMANN, Seattle, WA (US); William H. SHEFFLER, Seattle, WA (US); TJ BRUNETTE, Seattle, WA (US); Young-Jun PARK, Seattle, WA (US); Asim BERA, Seattle, WA (US); Matthew BICK, Seattle, WA (US); Rachel REDLER, Seattle, WA (US); Damian EKIERT, Seattle, WA (US); Gira BHABHA, Seattle, WA (US); David VEESLER, Seattle, WA (US); David BAKER, Seattle, WA (US)

(21) Appl. No.: 17/564,467

(22) Filed: Dec. 29, 2021

Related U.S. Application Data
(60) Provisional application No. 63/132,621, filed on Dec. 31, 2020.

(51) Int. Cl. C07K 14/47; A61K 47/62

(52) U.S. Cl. CPC C07K 14/47; C07K 2319/00; A61K 47/62

(57) ABSTRACT

The disclosure provides polypeptides as descried herein that including an amino acid sequence at least 50% identical to the amino acid sequence selected from the group consisting of SEQ ID NOS:1-46, oligomers of such polypeptides, methods for using such polypeptides and oligomers, and methods for designing such polypeptides and oligomers.

### CROSS-REFERENCE

This application claims priority to U.S. Provisional Application Ser. No. 63/132,621 filed Dec. 31, 2020, incorporated by reference herein in its entirety.

### FEDERAL FUNDING STATEMENT

This invention was made with government support under Grant Nos. HHSN272201700059C and R01 GM120553 and T32 GM007270 and T32 GM008268, awarded by the National Institutes of Health and Grant No. CHE-1629214, awarded by the National Science Foundation and Grant No. DE-SC0019288, awarded by the U.S. Department of Energy. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Nov. 29, 2021 having the file name “20-1456-US_SeqList_ST25.txt” and is 125 kb in size.

### BACKGROUND

Computational protein design has been used to create proteins that self-assemble into a wide variety of higher order structures. However, interface design remains challenging, and designable interface quality is heavily dependent on how well the building blocks complement each other during design. An alternative approach which avoids the need for designing new interfaces is to fuse oligomeric protein building blocks with helical linkers; however, lack of rigidity has made the structures of these assemblies difficult to precisely specify. More rigid junctions created by overlapping ideal helices and designing around the junction region has its own set of challenges in comparison to designing a new non-covalent protein-protein interface: first, for any pair of protein building blocks, there are far fewer positions for rigid fusion than are for unconstrained protein-protein docking limiting the space of possible solutions, and second, while in the non-covalent protein interface case the space searched can be limited by restricting building blocks to the symmetry axes of the desired nanomaterial, this is not possible in the case of rigid fusions, making the search more difficult as the number of building blocks increases.

### SUMMARY OF THE INVENTION

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-46 (Table 1), wherein residues in parentheses are optional. In one embodiment, amino acid changes from the reference polypeptide of any one of SEQ ID NOS:1-46 are conservative amino acid substitutions. In another embodiment, at least 1, 2, 3, 4, 5, 6, 7, 8, 9, 10, 11, 12, 13, 14, 15, 16, 17, 18, 19, 20, 21, 22, 23, 24, 25, or more of the non-polar residues in bold font in Table 1 are invariant relative to the reference polypeptide. In a further embodiment, at least 1, 2, 3, 4, 5, 6, 7, 8, 9, 10, 11, 12, 13, 14, 15, 16, 17, 18, 19, 20, 21, 22, 23, 24, 25, or more of the residues in bold font in Table 1 are invariant relative to the reference polypeptide. In another embodiment, the polypeptides further comprise an additional functional domain fused to the polypeptide, including but not limited to detectable proteins, purification tags, protein antigens, and protein therapeutics.

In another aspect, the disclosure provides nucleic acids encoding the polypeptide or fusion protein of any embodiment or combination of embodiments disclosed herein. In one aspect, the disclosure provides expression vectors comprising a nucleic acid of the disclosure operatively linked to a suitable control sequence. In a further aspect, the disclosure provides host cells comprising the polypeptide, nucleic acid, expression vector, and/or oligomer of any embodiment or combination of embodiments disclosed herein.

In one aspect, the disclosure provides oligomers, comprising two or more polypeptides or fusion proteins according to any embodiment or combination of embodiments disclosed herein. In one embodiment, the oligomer comprises a homo-oligomer. In another embodiment, the homo-oligomer comprises two or more identical polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-26, 39-40, and 45-46. In a further embodiment, the oligomer comprises a hetero-oligomer. In one embodiment, the hetero-oligomer comprises two different polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of:

SEQ ID NO: 27 and 29;

SEQ ID NO:27 and 30;

SEQ ID NO:28 and 29;

SEQ ID NO:28 and 30;

SEQ ID NO: 31 and 33;

SEQ ID NO:31 and 34;

SEQ ID NO:32 and 33;

SEQ ID NO:32 and 34;

SEQ ID NO: 35 and 37;

SEQ ID NO:35 and 38;

SEQ ID NO:36 and 37;

SEQ ID NO:36 and 38;

SEQ ID NO: 41 and 43;

SEQ ID NO:41 and 44;

SEQ ID NO:42 and 43; and

SEQ ID NO:42 and 44.

In another embodiment, the oligomer comprises a two-component dihedral assembly. In one embodiment, the two component dihedral assembly comprises two different polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of:

SEQ ID NO: 27 and 29;

SEQ ID NO:27 and 30;

SEQ ID NO:28 and 29;

SEQ ID NO:28 and 30;

SEQ ID NO: 31 and 33;

SEQ ID NO:31 and 34;

SEQ ID NO:32 and 33;

SEQ ID NO:32 and 34;

SEQ ID NO: 35 and 37;

SEQ ID NO:35 and 38;

SEQ ID NO:36 and 37; and

SEQ ID NO:36 and 38.

In a further embodiment, the oligomer comprises a one-component tetrahedral protein cage. In one embodiment, the one-component tetrahedral protein cage comprises the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:39 or 40. In another embodiment, the oligomer comprises a two-component icosahedral protein cage. In one embodiment, the two icosahedral protein cage comprises two different polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of:

SEQ ID NO: 41 and 43;

SEQ ID NO:41 and 44;

SEQ ID NO:42 and 43; and

SEQ ID NO:42 and 44.

The disclosure also provides compositions comprising the polypeptide, fusion protein, nucleic acid, expression vector, host cell, or oligomer of any embodiment or combination of embodiments disclosed herein. In one embodiment, the composition may further comprise a pharmaceutically acceptable carrier. In another embodiment, the composition may further comprise a therapeutic moiety or diagnostic moiety, for example, covalently attached to the oligomer. The disclosure also provides methods for using the polypeptide, fusion protein, nucleic acid, expression vector, host cell, oligomer, or composition of any embodiment or combination of embodiments disclosed herein for any suitable purpose, including but not limited to vaccine development, drug delivery and biomaterial production.

The disclosure further provides methods for designing multi-scale protein complexes by combinatorial assembly of oligomeric helical bundle and repeat protein building blocks, comprising any methods disclosed herein.

### DESCRIPTION OF THE FIGURES

FIG. 1. Overview of the rigid hierarchical fusion approach. (a) Hetero- and homo-oligomeric helical bundles are fused to de novo helical repeat proteins (left) to create a wide range of building blocks using HelixDock and HelixFuse (center). Symmetric units shown in grey. (b) Twenty representative HelixFuse outputs overlaid in groups of five to display the wide range of diversity that can be generated by using a single helical bundle core. (c) These are then further assembled into higher ordered structures through helical fusion (WORMS, right). The examples shown are cyclic crowns (top), dihedral rings (middle), and icosahedral nanocages (bottom).

FIG. 2. Homo-oligomer diversification by repeat protein fusion. Central oligomer units and fused DHRs are shown. Design of (a) C3 HD-1069, (b) C3_HF_Wm-0024A, and (c) C3_nat_HF-0005. Overlay of the design model and crystal structure shows the overall match of the backbone. Inset shows the correct placement of the rotamers in the designed junction region. Design of higher order oligomer fusions (d) C4_nat_HF-7900 and (e) C5_HF-3921 as characterized by cryo-EM. C4_nat_HF-7900 design model and Cryo-EM map, with inset highlighting the high resolution (˜3.8 Å) density. C5_HF-3921 inset showing density surrounding the designed junction. (f) C5_HF-2101, (g) C5_HF-0019, (h) C6_HF-0075, and (i) C6_HF-0080 showed good overall match to its negative-stain EM 2D class averages (top) from one direction; predicted projection map for comparison on the bottom.

FIG. 3. Design of cyclic “crown” (Crn) structures from heterodimeric building blocks. (a) Hetero-dimeric HB fused with different DHRs were fused together using WORMS by enforcing a specific overall cyclic symmetry (C3 and C5 shown). (b) The backbones of the crystal structure of C3_Crn-05 overlaid with the design model. Insets show the backbone matching focused at each of the fusion locations. (c) A C5 crown (C5_Crn-07, asymmetric unit) was fused to DHR units on either exterminal (“C5_Crn_HF-12”, arrow) or internal termini (“C5_Crn_HF-26”, dark arrow). The two structures were then merged together to generate a double fusion (“C5_Crn_HF-12_26”, darkest arrow). (d) Cryo-EM class average of the fused 12_26 structure; the major C5 species shown. 3D reconstruction shows the main features of the designed structure are present, as is also evident in the class average (right).

FIG. 4. Design of two-component dihedral rings using WORMS (Wm). (a) Two different homodimeric HBs with DHR extensions were aligned to their respective symmetrical axes with dihedral symmetry. An additional heterodimer was placed between them and systematically scanned and fused together to design an 8-chain D2 ring. (b) The final asymmetric unit shown while the inset preserves the original. (c) Negative-stain EM followed by 2D average and 3D reconstruction of D2_Wm-01 and D2_Wm-01_trunc show that the major features of the designs were recapitulated (left) designed model, (middle) overlay of the designed models with the 3D reconstructions, (right) 2D averages.

FIG. 5. Design of assemblies with point group symmetry through helical fusion with WORMS. (a) Tetrahedron design schematic. A HB and a C2 homo-oligomeric made from ankyrin repeat proteins were aligned to their respective tetrahedral symmetry axis, and connected via fusion to Ankyrin repeat monomers to generate the target architecture. (b) 3D reconstruction reveals a well-fitting map of T_Wm-1606. (c) Icosahedral design schematic. Libraries of unverified cyclic fusion homo-dimers and trimers were aligned to the corresponding icosahedral symmetry axes. Using WORMS, fusions to DHRs split in the center that hold the two homo-oligomers in the orientations which generate icosahedral structures were identified. (d) Cryo-EM 3D reconstruction of I32_Wm-42 closely matches the designed model.

FIG. 6. SEC and SAXS characterizations of C2 symmetric oligomers which were designed using the HelixDock protocol. The left panel shows the designed models; the middle shows the SEC curves (Superdex™ 200), and the right shows the SAXS fitting comparison between the designed model and the experimental data.

FIG. 7A. SEC and SAXS characterizations of C3 symmetric oligomers which were designed using the HelixDock protocol. The left panel shows the designed models; the middle shows the SEC curves (Superdex™ 200), and the right shows the SAXS fitting comparison between the designed model and the experimental data.

FIG. 7B. SEC and SAXS characterizations of C3 symmetric oligomers which were designed using the HelixDock protocol. The left panel shows the designed models; the middle shows the SEC curves (Superdex™ 200), and the right shows the SAXS fitting comparison between the designed model and the experimental data.

FIG. 8, SEC and SAXS characterizations of C6 symmetric oligomers which were designed using the HelixDock protocol. The left panel shows the designed models; the middle shows the SEC curves (Superdex™ 200), and the right shows the SAXS fitting comparison between the designed model and the experimental data.

FIG. 9. SEC and SAXS characterizations of C5 symmetric oligomers which were designed using the HelixFuse protocol. The left panel shows the designed models; the middle shows the SEC curves (C5_HF-2101 and C5_HF-3921 by Superose™ 6, remaining by Superdex™ 200), and the right shows the SAXS fitting comparison between the designed model and the experimental data.

FIG. 10. SEC and SAXS characterizations of C4 and C6 symmetric oligomers which were designed using the HelixFuse protocol. The left panel shows the designed models; the middle shows the SEC curves (C4_nat_HF-7900 by Superose™ 6, C6_HF-0069 and C6_HF-0080 by Superdex™ 200), and the right shows the SAXS fitting comparison between the designed model and the experimental data.

FIG. 11. Cryo-EM data and reconstruction for C4_nat_HF-7900. (A) Representative motion-corrected micrograph. (B) Representative 2D class averages. (C) Locally-filtered cryo-EM map colored by local resolution. (D) Fit of cryo-EM structure (sticks) to density (mesh) in areas of high, intermediate, and low local resolution.

FIG. 12. Cryo-EM data processing workflow for C4_nat_HF-7900.

FIG. 13. Cryo-EM data for C5_HF-3921. (A) Representative motion-corrected micrograph. (B) Representative 2D class averages. (C) Cryo-EM map colored by local resolution.

FIG. 14. Cryo-EM data processing workflow of C5_HF-3921.

FIG. 15. Negative stain EM data for C5_HF-2101. (A) Representative micrograph. (B) Most populated 2D class averages; numbers on each class image indicate the number of particles in that class. (C) 2D projections of a 20 Å-filtered volume generated from the atomic coordinates of the design model. (D) Selected 2D class averages shown alongside matching model projections.

FIG. 16. Negative stain EM data for C5_HF-0007. (A) Representative micrograph. (B) Most populated 2D class averages; numbers on each class image indicate the number of particles in that class. (C) 2D projections of a 20 Å-filtered volume generated from the atomic coordinates of the design model. (D) Selected 2D class average shown alongside matching model projection.

FIG. 17. Cryo-EM data for C5_HF-0019. (A) Representative motion-corrected micrograph. (B) Most populated 2D class averages; numbers on each class image indicate the number of particles in that class. (C) 2D projections of a 15 Å-filtered volume generated from the atomic coordinates of the design model. (D) Selected 2D class average shown alongside matching model projection.

FIG. 18. Negative stain EM data for C6_HF-0075. (A) Representative micrograph. (B) Most populated 2D class averages; numbers on each class image indicate the number of particles in that class. (C) 2D projections of a 20 Å-filtered volume generated from the atomic coordinates of the design model. (D) Selected 2D class average shown alongside matching model projection.

FIG. 19. Negative stain EM data for C6_HF-0080. (A) Representative micrograph. (B) Most populated 2D class averages; numbers on each class image indicate the number of particles in that class. (C) 2D projections of a 20 Å-filtered volume generated from the atomic coordinates of the design model. (D) Selected 2D class average shown alongside matching model projection.

FIG. 20. Alignment of the original scaffolds to C3_nat_HF-0005's crystal structure and C4_nat_HF-7900's cryo-EM model. Symmetric units hidden for clarity. A) C3_nat_HF-0005 design mode and crystal structure, aligned at the 1wa3 hub. DHR49 model and DHR49's original crystal structure aligned at the junction helix. B) A small deviation in the loop region of the first helix (darkest arrow) propagates into a large deviation towards the distal portion of the protein (lighter arrows). C) tpr1C4_pm3 aligned to C4_nat_HF-7900's cryo-EM model. D) DHR79 aligned to C4_nat_HF-7900's cryo-EM model. While the majority of the DHR aligns well, the N-terminal helices align less well to the model regardless of the new fusion region (arrow). The C-terminal helix is not present in the cryo-EM map (arrow).

FIG. 21. Characterization of C3 and C5 crowns. SEC of A) C3_Crn-05 (Superdex™ 200), and B) C5_Crn-07 (Superdex™ 200). C) C5_Crn-07 negative stain micrograph, D) C5_Cm-07 negative stain 2D average showing all alternative states.

FIG. 22. Characterization of C5_Crn-07 with extended arms. SEC of A) C5_Cm_HF-12 (Superose™ 6), B) C5_Cm_HF-26 (Superose™ 6), and C) C5_Crn_HF-12_26 (Superose™ 6). Arrows indicate the correct elution fractions; aggregate fraction for A and B were disregarded. Cryo electron microscopy characterization of C5_Crn_HF-12_26. D) Representative micrograph; E) class averages showing off-target states. Cryo-EM density maps for additional off-target states: F) C6 (left—top view, right—side view), G) D5 (left—top view, right—side view).

FIG. 23. Characterization of D2_Wm-01 (A) and D2_Wm-01_trunc (B) dihedral rings. The left panel shows the designed models; the middle panel shows the SEC curves (Superose™ Increase 10/300 S6 column); and the right panel shows SAXS fitting curves which were compared between the designed model and the experimental data.

FIG. 24. Characterization of D2_Wm-02 dihedral ring. Two-component D2_Wm-02 ring was designed using the WORMS protocol, which was then expressed and subsequently purified using SEC (Superose™ Increase 10/300 S6 column). Purified protein was characterized by either SAXS or NS EM. 2D average of the NS EM shows features resembling the designed model, and the 3D density map (upper right) overlays accurately with the designed model. Likewise, SAXS fitting (lower right) shows the close resemblance between the designed model and the experimental data.

FIG. 25. SEC, SAXS, and Negative stain characterization of T_Wm-1606 tetrahedron. A) SEC, B) SAXS, C) Representative micrograph, and D) 2D class averages.

FIG. 26. SEC and Cryo electron microscopy characterization of I32_Wm-42 icosahedral nanocage. SEC of I32_Wm-42 A) after Ni-NTA purification (Superose™ 6), and B) collected void fraction from A (arrow) to re-run on Sephacryl™ 500. Fractions ˜15 mL were collected for further analysis (arrow). C) Representative micrograph; D) class averages.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-46 (see Table 1), wherein residues in parentheses are optional.

TABLE 1

| | |
|---|---|
| HelixDock | NDEKEKLKELLKRAEELAKSPDPEDLKEAVRLAEEVVRERPGSNLAKKALEIILRAAEELAK |
| >C3_HD-1069 | LPDPKALIAAVLAAIKVVREQPGSNLAKKALEIILRAAEELAKLPDPLALAAAVVAATIVVL |
| | TQPGSELAKKALEIIERAAEELKKSPDPLAQLLAIAAEALVIALKSSSEETIKEMVKLITLA |
| | LLTSLLILILILLDLKEMLERLEKNPDKDVIVKVLKVIVKAIEASVLNQAISAINQILLALS |
| | D |
| | (SEQ ID NO: 1) |
| | (MGHHHHHHGG)NDEKEKLKELLKRAEELAKSPDPEDLKEAVRLAEEVVRERPGSNLAKKAL |
| | EIILRAAEELAKLPDPKALIAAVLAAIKVVREQPGSNLAKKALEIILRAAEELAKLPDPLAL |
| | AAAVVAATIVVLTQPGSELAKKALEIIERAAEELKKSPDPLAQLLAIAAEALVIALKSSSEE |
| | TIKEMVKLTTLALLTSLLILILILLDLKEMLERLEKNPDKDVIVKVLKVIVKAIEASVLNQA |
| | ISAINQILLALSD |
| | (SEQ ID NO: 2) |
| HelixFuse | SEEEQERIRRILKEARKSGTEESLRQAIEDVAQLAKKSQDSEVLEEAIRVILRIAKESGSEE |
| >C3_nat_HF- | ALRQAIRAVAEIAKEAQDSEVLEEAIRVILRIAKESGSEEALRQALRAVAEIAEEAKDERVR |
| 0005 | KEAVRVMLQIAKESGSKEAVKLAFEMILRVVRIIAVLRANSVEEAKEKALAVFEGGVLAIEI |
| | TFTVPDADTVIKELSFLEKEGAIIGAGTVISVEQCRKAVESGALFIVSPHLDEEISQFCDEA |
| | GVAYAPGVMTPTELVKAMKLGHRILKLFPGEVVGPQFVKAMKGPFPNVRFVPTGGVNLDNVA |
| | EWFKAGVLAVGVGSALVKGTPDEVREKAKAFVEKIKAA (SEQ ID NO: 3) |
| | (MGHHHHHHGGS)SEEEQERIRRILKEARKSGTEESLRQAIEDVAQLAKKSQDSEVLEEAIR |
| | VILRIAKESGSEEALRQAIRAVAEIAKEAQDSEVLEEAIRVILRIAKESGSEEALRQALRAV |
| | AEIAEEAKDERVRKEAVRVMLQIAKESGSKEAVKLAFEMILRVVRIIAVLRANSVEEAKEKA |
| | LAVFEGGVLAIEITFTVPDADTVIKELSFLEKEGAIIGAGTVISVEQCRKAVESGALFIVSP |
| | HLDEEISQFCDEAGVAYAPGVMTPTELVKAMKLGHRILKLFPGEVVGPQFVKAMKGPFPNVR |
| | FVPIGGVNLDNVAEWFKAGVLAVGVGSALVKGTPDEVREKAKAFVEKIKAA (SEQ ID |
| | NO: 4) |
| >C4_nat_HF- | ASSWVMLGLLLSLLNRLSLAAEAYKKAIELDPNDALAWLLLGSVLLLLGREEEAEEAARKAI |
| 7900 | ELKPEMDSARRLEGIIELIRRAREAAERAQEAAERTGDPRVRELARELKRLAQEAAEEVRRD |
| | PDSKDVNEALKLIVEAIEAAVRALEAAERTGDPEVRELARELVRLAVEAAEEVQRNPSSSDV |
| | NEALKLIVEAIDAAVRALEAAEKTGDPEVRELARELVRLAVEAAEEVQRNPSSEEVNEALKD |
| | IVKAIQEAVESLREAEESGDPEKREKARERVREAVERAEEVQRDPSS (SEQ ID NO: 5) |
| | (M)ASSWVMLGLLLSLLNRLSLAAEAYKKAIELDPNDALAWLLLGSVLLLLGREEEAEEAAR |
| | KAIELKPEMDSARRLEGIIELIRRAREAAERAQEAAERTGDPRVRELARELKRLAQEAAEEV |
| | RRDPDSKDVNEALKLIVEAIEAAVRALEAAERTGDPEVRELARELVRLAVEAAEEVQRNPSS |
| | SDVNEALKLIVEAIDAAVRALEAAEKTGDPEVRELARELVRLAVEAAEEVQRNPSSEEVNEA |
| | LKDIVKAIQEAVESLREAEESGDPEKREKARERVREAVERAEEVQRDPSS(GGSWGLEHHHH |
| | HH) (SEQ ID NO: 6) |
| >C5_HF-3921 | SDLQEVADRIVEQLKREGRSPEEARKEARRLIEEIKQSAGGDSELIEVAVRIVKELEEQGRS |
| | PSEAAKEAVELIERIRRAAGGDSELIEVAVRIVKELEEQGRSPSEAAKEAVELIERIRRAAG |
| | GDSELIEVAVRIVKELEEQGRSPSEAAKEAVELIERIRRAAGGDSELIEVAVRIVKELEEQG |
| | RSPSEAAKEAVELIERIRRAAGGDSELIEVAVRIVKFLEEAGMSPSEAAKVAVELIERIRRA |
| | AGGDSELIEKAVRIVRRLERRGLSPAEAAKIAVAIIAAEVLSREAEKIREETEEVKKEIEES |
| | KKRPQSESAKNLILIMQLLINQIRLLALQIQMLRLQLEL (SEQ ID NO: 7) |
| | (MGHHHHHHGSGSENLYFQGGS)SDLQEVADRIVEQLKREGRSPEEARKEARRLIEEIKQSA |
| | GGDSELIEVAVRIVKELEEQGRSPSEAAKEAVELIERIRRAAGGDSELIEVAVRIVKELEEQ |
| | GRSPSEAAKEAVELIERIRRAAGGDSELIEVAVRIVKELEEQGRSPSEAAKEAVELIERIRR |
| | AAGGDSELIEVAVRIVKELEEQGRSPSEAAKEAVELIERIRRAAGGDSELIEVAVRIVKFLE |
| | EAGMSPSEAAKVAVELIERIRRAAGGDSELIEKAVRIVRRLERRGLSPAEAAKIAVAIIAAE |
| | VLSREAEKIREETEEVKKEIEESKKRPQSESAKNLILIMQLLINQIRLLALQIQMLRLQLEL |
| | (SEQ ID NO: 8) |
| >C5_HF-2101 | SEKEKVEELAQRIREQLPDTELAREAQELADEARKSDDSEALKVVYLALRIVQQLPDTELAR |
| | EALELAKEAVKSTDSEALKVVYLALRIVQQLPDTELAREALELAKEAVKSTDSEALKVVYLA |
| | LRIVQQLPDTELAREALELAKEAVKSTDSEALKVVYLALRIVQQLPDTELAREALELAKEAV |
| | KSTDSEALKVVYLALRIVQQLPDTELAREALELAKEAVKSTDSEALKVVYLALRIVQLLPDT |
| | DLARKALELAKEAVKMDDQEVLKVVYKALQIVADKPNTEEADEALRDARLKLEAARLRREME |
| | KIREETEEVKKEIEESKKRPQSESAKNLILIMQLLINQIRLLALQIRMLDLQLKL (SEQ ID |
| | NO: 9) |
| | (MGHHHHHHGSGSENLYFQGGS)SEKEKVEELAQRIREQLPDTELAREAQELADEARKSDDS |
| | EALKVVYLALRIVQQLPDTELAREALELAKEAVKSTDSEALKVVYLALRIVQQLPDTELARE |
| | ALELAKEAVKSTDSEALKVVYLALRIVQQLPDTELAREALELAKEAVKSTDSEALKVVYLAL |
| | RIVQQLPDTELAREALELAKEAVKSTDSEALKVVYLALRIVQQLPDTELAREALELAKEAVK |
| | STDSEALKVVYLALRIVQLLPDTDLARKALELAKEAVKMDDQEVLKVVYKALQIVADKPNTE |
| | EADEALRDARLKLEAARLRREMEKIREETEEVKKEIEESKKRPQSESAKNLILIMQLLINQI |
| | RLLALQIRMLDLQLKL (SEQ ID NO: 10) |
| >C5_HF-0019 | NDEKEKLKELLKRAEELAKSPDPEDLKEAVRLAEEVVRERPGSNLAKKALEIILRAAEELAK |
| | LPDPEALKEAVKAAEKVVREQPGSNLAKKAQEIILRAAEELAKLEDEEALKEAIKAAEKVIE |
| | LEPGSELAKEAKRIIEKAAKMLADILRKEMEKIREETEEVKKEIEESKKRPQSESAKNLILI |
| | MQLLINQIRLLALQIRMLVLQLIL (SEQ ID NO: 11) |
| | (MGHHHHHHGGSENLYFQSGG)NDEKEKLKELLKRAEELAKSPDPEDLKEAVRLAEEVVRER |
| | PGSNLAKKALEIILRAAEELAKLPDPEALKEAVKAAEKVVREQPGSNLAKKAQEIILRAAEE |
| | LAKLEDEEALKEAIKAAEKVIELEPGSELAKEAKRIIEKAAKMLADILRKEMEKIREETEEV |
| | KKEIEESKKRPQSESAKNLILIMQLLINQIRLLALQIRMLVLQLIL (SEQ ID NO: 12) |
| >C6_HF-0075 | SIQEKAKQSVIRKVKEEGGSEEEARERAKEVEERLKKEADDSTLVRAAAAVVLYVLEKGGST |
| | EEAVQRAREVIERLKKEASDSTLVRAAAAVVLYVLEKGGSTEEAVQRAREVIERLKKEASDS |
| | TLVRAAAAVVLYVLEKGGSTEEAVQRAREVIERLKKEASDSTLVRAAAAVVLYVLEKGGSTE |
| | EAVQRAREVIERLKKEASDSTLVRAAAAVVLYVLEKGGSTEEAVQRAREVIERLKKEASDST |
| | LVRAAAAVVLYVLEKGGSTEEAVDRAREVIEALKKFANDEEEIRRAAKVVLKVLETGGSVEE |
| | AMIRAALEILLDMLKEAAKKLKKLEDKIRRSEEISKTDDDPKAQSLQLIAESLMLIAESLLI |
| | IAISLLLSSLAG (SEQ ID NO: 13) |
| | (MGHHHHHHGWSG)SIQEKAKQSVIRKVKEEGGSEEEARERAKEVEERLKKEADDSTLVRAA |
| | AAVVLYVLEKGGSTEEAVQRAREVIERLKKEASDSTLVRAAAAVVLYVLEKGGSTEEAVQRA |
| | REVIERLKKEASDSTLVRAAAAVVLYVLEKGGSTEEAVQRAREVIERLKKEASDSTLVRAAA |
| | AVVLYVLEKGGSTEEAVQRAREVIERLKKEASDSTLVRAAAAVVLYVLEKGGSTEEAVQRAR |
| | EVIERLKKEASDSTLVRAAAAVVLYVLEKGGSTEEAVDRAREVIEALKKFANDEEEIRRAAK |
| | VVLKVLETGGSVEEAMIRAALEILLDMLKEAAKKLKKLEDKIRRSEEISKTDDDPKAQSLQL |
| | IAESLMLIAESLLIIAISLLLSSLAG (SEQ ID NO: 14) |
| >C6_HF-0080 | STKEKARQLAEEAKETAEKVGDPELIKLAEQASQEGDSEKAKAILLAAEAARVAKEVGDPEL |
| | IKLALEAARRGDSEKAKAILLAAEAARVAKEVGDPELIKLALEAARRGDSEKAKAILLAAEA |
| | ARVAKEVGDPELIKLALEAARRGDSEKAKAILLAAEAARVAKEVGDPELIKLALEAARRGDS |
| | EKAKAILLAAEAARVAKEVGDPELIKLALEAARRGDSEKAKAILLAAEAARVAKEAGIPEMI |
| | KAALRAARLGASDAAQAILEAADEARKAREEGDKKKEKSAELKALLALAKVKLKRLEDKIRR |
| | SEEISKTDDDPKAQSLQLIAESLMLIAESLLIIAISLLLSSDAG (SEQ ID NO: 15) |
| | (MGHHHHHHGWSG)STKEKARQLAEEAKETAEKVGDPELIKLAEQASQEGDSEKAKAILLAA |
| | EAARVAKEVGDPELIKLALEAARRGDSEKAKAILLAAEAARVAKEVGDPELIKLALEAARRG |
| | DSEKAKAILLAAEAARVAKEVGDPELIKLALEAARRGDSEKAKAILLAAEAARVAKEVGDPE |
| | LIKLALEAARRGDSEKAKAILLAAEAARVAKEVGDPELIKLALEAARRGDSEKAKAILLAAE |
| | AARVAKEAGIPEMIKAALRAARLGASDAAQAILEAADEARKAREEGDKKKEKSAELKALLAL |
| | AKVKLKRLEDKIRRSEEISKTDDDPKAQSLQLIAESLMLIAESLLIIAISLLLSSDAG |
| | (SEQ ID NO: 16) |
| Crowns | |
| >C3_Crn-05 | GDRSDHAKKLKTFLENLRRHLDRLDKHIKQLRDILSENPEDERVKDVIDLSERSVRIVKIVI |
| | KIFEDSVRKLLKQINKEAEELAKSPDPEDLKRAVELAEAVVRADPGSNLSKKALEIILRAAA |
| | ELAKLPDPDALAAAARAASKVQQEQPGSNLAKAAQEIMRQASRAAEEAARRAKETLEKAEKD |
| | GDPETALKAVETVVKVARALNQIATMAGSEEAQERAARVASEAARLAERVLELAEKQGDPEV |
| | ARRARELQEKVLDILLDILEQILQTATKIIDDANKLLEKLRRSERKDPKVVETYVELLKRHE |
| | RLVKQLLEIAKAHAEAVE (SEQ ID NO: 17) |
| | (M)GDRSDHAKKLKTFLENLRRHLDRLDKHIKQLRDILSENPEDERVKDVIDLSERSVRIVK |
| | TVIKIFEDSVRKLLKQINKEAEELAKSPDPEDLKRAVELAEAVVRADPGSNLSKKALEIILR |
| | AAAELAKLPDPDALAAAARAASKVQQEQPGSNLAKAAQEIMRQASRAAEEAARRAKETLEKA |
| | EKDGDPETALKAVETVVKVARALNQIATMAGSEEAQERAARVASEAARLAERVLELAEKQGD |
| | PEVARRARELQEKVLDILLDILEQILQTATKIIDDANKLLEKLRRSERKDPKVVETYVELLK |
| | RHERLVKQLLEIAKAHAEAVE(GGSLEHHHHHH) (SEQ ID NO: 18) |
| >C5_Crn-07 | GDRSEHAKKLKTFLENLRRHLDRLDKHIKQLRDILSENPEDERVKDVIDLSERSVRIVKTVI |
| | KIFEDSVRKLEKQILKEAEELAKSPDPEDLKRAVELARAVIEANPGSNLSRKAMEIIERAAR |
| | ELSKLPDPEAQRTAIEAASQLATMAAATGNTDQVRRAAELMKEIARLAGTEEAKDLALDALL |
| | DVLETALQIATKIIDDANKLLEKLRRSERKDPKVVETYVELLKRHEEAVRLLLEVAKTHADI |
| | VE (SEQ ID NO: 19) |
| | (M)GDRSEHAKKLKTFLENLRRHLDRLDKHIKQLRDILSENPEDERVKDVIDLSERSVRIVK |
| | TVIKIFEDSVRKLEKQILKEAEELAKSPDPEDLKRAVELARAVIEANPGSNLSRKAMEIIER |
| | AARELSKLPDPEAQRTAIEAASQLATMAAATGNTDQVRRAAELMKEIARLAGTEEAKDLALD |
| | ALLDVLETALQIATKIIDDANKLLEKLRRSERKDPKVVETYVELLKRHEEAVRLLLEVAKTH |
| | ADIVE(GGSLEHHHHHH) (SEQ ID NO: 20) |
| >C5_Crn_HF-12 | GDRSEHAKKLKTFLENLRRHLDRLDKHIKQLRDILSEHPHDERVKDVIDLSERSVRIVKKVIKIFEDSV |
| | RELEKMILKEAEELAKSPDPEDLKRAVELARAVIEANPGSNLSRKAMEIIERAARELSKLPDPEAQRTA |
| | IEAASQLATMAAATGNTDQVRRAAKLMMRIAILAGTEEASDLALDALLDVLETALQIATKIIDDANKLL |
| | EKLRRSHHHDPKVVETYVELLKRHEEAVRLLLDVAIMHALIVVMQDAIEAAREGDKDRARKALQDALEL |
| | ARLAGTTEAVEAALLVVEAVAVAAARAGATDVVREALEVALEIARESGTTEAVKLALEVVASVAIEAAR |
| | RGNTDAVREALEVALEIARESGTEEAVRLALEVVKRVSDEAKKQGNEDAVKEAEEVRKKIEEES (SEQ |
| | ID NO: 21) |
| | (M)GDRSEHAKKLKTFLENLRRHLDRLDKHIKQLRDILSEHPHDERVKDVIDLSERSVRIVKKVIKIFE |
| | DSVRELEKMILKEAEELAKSPDPEDLKRAVELARAVIEANPGSNLSRKAMEIIERAARELSKLPDPEAQ |
| | RTAIEAASQLATMAAATGNTDQVRRAAKLMMRIAILAGTEEASDLALDALLDVLETALQIATKIIDDAN |
| | KLLEKLRRSHHHDPKVVETYVELLKRHEEAVRLLLDVAIMHALIVVMQDAIEAAREGDKDRARKALQDA |
| | LELARLAGTTEAVEAALLVVEAVAVAAARAGATDVVREALEVALEIARESGTTEAVKLALEVVASVAIE |
| | AARRGNTDAVREALEVALEIARESGTEEAVRLALEVVKRVSDEAKKQGNEDAVKEAEEVRKKIEEES |
| | (SEQ ID NO: 22) |
| >C5_Crn_HF-26 | GTESKVLEAEMSIKKAEWSAREGNPEKATEDLMRAMLLIRELDVLAQKTGSAEVLVKAAALAEKLAKVA |
| | REVGDPEMAREAEKLARALAAKLLSMHAKLLATFLENLRRHLDRLDKHIKQLRDILSEHPHDERVKDVI |
| | DLSERSVRIVKTVIKIFEDSVRKLLKEMLKRAEELAKSPDPLDLKAAVDVARAVIEANPGSNLSRKAME |
| | IIERAARELSKLPDPLAIATAIEAASQLATMAAATGNTDQVRRAAELMKEIARLAGTDLAKAAALLALL |
| | RVLETALQIATKIIDDANKLLEKLRRSHHHDPKVVETYVELLKRHEEAVRLLLEVAKTHADIVE (SEQ |
| | ID NO: 23) |
| | (M)GTESKVLEAEMSIKKAEWSAREGNPEKATEDLMRAMLLIRELDVLAQKTGSAEVLVKAAALAEKLA |
| | KVAREVGDPEMAREAEKLARALAAKLLSMHAKLLATFLENLRRHLDRLDKHIKQLRDILSEHPHDERVK |
| | DVIDLSERSVRIVKTVIKIFEDSVRKLLKEMLKRAEELAKSPDPLDLKAAVDVARAVIEANPGSNLSRK |
| | AMEIIERAARELSKLPDPLAIATAIEAASQLATMAAATGNTDQVRRAAELMKEIARLAGTDLAKAAALL |
| | ALLRVLETALQIATKIIDDANKLLEKLRRSHHHDPKVVETYVELLKRHEEAVRLLLEVAKTHADIVE |
| | (SEQ ID NO: 24) |
| >C5_Crn_HF- | GTESKVLEAEMSIKKAEWSAREGNPEKATEDLMRAMLLIRELDVLAQKTGSAEVLVKAAALAEKLAKVA |
| 12_26 | REVGDPEMAREAEKLARALAAKLLSMHAKLLATFLENLRRHLDRLDKHIKQLRDILSEHPHDERVKDVI |
| | DLSERSVRIVKKVIKIFEDSVRELLKMMLKRAEELAKSPDPEDLKAAVDVARAVIEANPGSNLSRKAME |
| | IIERAARELSKLPDPEAIATAIEAASQLATMAAATGNTDQVRRAAKLMMRIAILAGTDLASAAALDALL |
| | RVLETALQIATKIIDDANKLLEKLRRSHHHDPKVVETYVELLKRHEEAVRLLLDVAIMHALIVVMQDAI |
| | EAAREGDKDRARKALQDALELARLAGTTEAVEAALLVVEAVAVAAARAGATDVVREALEVALEIARESG |
| | TTEAVKLALEVVASVAIEAARRGNTDAVREALEVALEIARESGTEEAVRLALEVVKRVSDEAKKQGNED |
| | AVKEAEEVRKKIEEES (SEQ ID NO: 25) |
| | (M)GTESKVLEAEMSIKKAEWSAREGNPEKATEDLMRAMLLIRELDVLAQKTGSAEVLVKAAALAEKLA |
| | KVAREVGDPEMAREAEKLARALAAKLLSMHAKLLATFLENLRRHLDRLDKHIKQLRDILSEHPHDERVK |
| | DVIDLSERSVRIVKKVIKIFEDSVRELLKMMLKRAEELAKSPDPEDLKAAVDVARAVIEANPGSNLSRK |
| | AMEIIERAARELSKLPDPEAIATAIEAASQLATMAAATGNTDQVRRAAKLMMRIAILAGTDLASAAALD |
| | ALLRVLETALQIATKIIDDANKLLEKLRRSHHHDPKVVETYVELLKRHEEAVRLLLDVAIMHALIVVMQ |
| | DAIEAAREGDKDRARKALQDALELARLAGTTEAVEAALLVVEAVAVAAARAGATDVVREALEVALEIAR |
| | ESGTTEAVKLALEVVASVAIEAARRGNTDAVREALEVALEIARESGTEEAVRLALEVVKRVSDEAKKQG |
| | NEDAVKEAEEVRKKIEEES (SEQ ID NO: 26) |
| Dihedral | |
| rings | |
| >D2_Wm-01A | GTREESLKEQLRSLREQAELAARLLRLLKELERLQREGSSDEDVRELLREIKELVAEIIKLI |
| | MEQLLLIAEQLLGRSEAAELALRAIRLALELCRQSTDLEECLRLLKTAIKALENALRHPDST |
| | TAKARLMAITARLLAQQLRTQHPDSQAARDAEKLADQAERAVRLATRLYEEHPNAEISEMCS |
| | QAAYAAALMASIAAILAQRHPDSQIARDLIRLASELAEMVKRMCER (SEQ ID NO: 27) |
| | (M)GTREESLKEQLRSLREQAELAARLLRLLKELERLQREGSSDEDVRELLREIKELVAEII |
| | KLIMEQLLLIAEQLLGRSEAAELALRAIRLALELCRQSTDLEECLRLLKTAIKALENALRHP |
| | DSTTAKARLMAITARLLAQQLRTQHPDSQAARDAEKLADQAERAVRLATRLYEEHPNAEISE |
| | MCSQAAYAAALMASIAAILAQRHPDSQIARDLIRLASELAEMVKRMCER(GGSWGLEHHHHH |
| | H) (SEQ ID NO: 28) |
| >D2_Wm-01B | GTREELAKELLRSLREQAESLARQLRLLKELERLQREGSSDEDVRELLREIKELAAEQIKLI |
| | MEQLLLIAELTLGRSEAAELALDAIRQALEACRTMDNQEACTRLLKLAIQMLELATRAPDAE |
| | AAKLALEAAKKAIELANRHPGSQAAEDATKLAQQAMEAVRLALKLYEEHPNADIADLCRRAA |
| | AEAAEAASKAAELAQRHPDSQAARDAIKLASQAAEAVKLACELAQEHPNADKAKLCILLASA |
| | AALLASIAAMLAQRHPDSQEARDMIRIASELAELVKEICER (SEQ ID NO: 29) |
| | (M)GTREELAKELLRSLREQAESLARQLRLLKELERLQREGSSDEDVRELLREIKELAAEQI |
| | KLIMEQLLLIAELTLGRSEAAELALDAIRQALEACRTMDNQEACTRLLKLAIQMLELATRAP |
| | DAEAAKLALEAAKKAIELANRHPGSQAAEDATKLAQQAMEAVRLALKLYEEHPNADIADLCR |
| | RAAAEAAEAASKAAELAQRHPDSQAARDAIKLASQAAEAVKLACELAQEHPNADKAKLCILL |
| | ASAAALLASIAAMLAQRHPDSQEARDMIRIASELAELVKEICER (SEQ ID NO: 30) |
| D2_Wm-02A | GTREEIIRELARSLAEQAELTARLERSLREQERLQREGSSDEDVRELIREQKELVREILKLI |
| | AEQILLIAELLLASTRSEAAELALRAIRNAIEACKNADNEEMCRQLMRMAQNALELATQAPD |
| | AEAAKAALRAIDLAVELASRHPGSQAADDALKLAQQAAEAVKLALDLYREHPNADIADLCRK |
| | AAKEAAEAASKAAELAQRHPDSQAARDAIKLASQAAEAVKLACELAQEHPNAEIAKMCILAA |
| | SAAALMASIAAILAQRHPDSQIARDLIRLASELAEMVKRMCER (SEQ ID NO: 31) |
| | (M)GTREEIIRELARSLAEQAELTARLERSLREQERLQREGSSDEDVRELIREQKELVREIL |
| | KLIAEQILLIAELLLASTRSEAAELALRAIRNAIEACKNADNEEMCRQLMRMAQNALELATQ |
| | APDAEAAKAALRAIDLAVELASRHPGSQAADDALKLAQQAAEAVKLALDLYREHPNADIADL |
| | CRKAAKEAAEAASKAAELAQRHPDSQAARDAIKLASQAAEAVKLACELAQEHPNAEIAKMCI |
| | LAASAAALMASIAAILAQRHPDSQIARDLIRLASELAEMVKRMCER(GGSWGLEHHHHHH) |
| | (SEQ ID NO: 32) |
| >D2_Wm-02B | GTREELAKELLRSLREQAESLARQLRLLKELERLQREGSSDEDVRELLREIKELAAEQIKLI |
| | MEQLLLIAELMLGRSEAAELALEAIRLALELCRQSTDQEQCTDLLRQATEALETATRYPDDT |
| | NAKAKLMAITARLLAQQLRTQHPDSQAARDAEKLADQAEKAVRLAKRLYEEHPNADKSELCS |
| | QLAYAAALLASIAAMLAQRHPDSQEARDMIRIASELAELVKEICER (SEQ ID NO: 33) |
| | (M)GTREELAKELLRSLREQAESLARQLRLLKELERLQREGSSDEDVRELLREIKELAAEQI |
| | KLIMEQLLLIAELMLGRSEAAELALEAIRLALELCRQSTDQEQCTDLLRQATEALETATRYP |
| | DDTNAKAKLMAITARLLAQQLRTQHPDSQAARDAEKLADQAEKAVRLAKRLYEEHPNADKSE |
| | LCSQLAYAAALLASIAAMLAQRHPDSQEARDMIRIASELAELVKEICER (SEQ ID |
| | NO: 34) |
| >D2_Wm- | GTREESLKEQLRSLREQAELAARLLRLQREGSSDEDVKELVAEIIKLIMEQLLLIAEQLLGR |
| 01_truncA | SEAAELALRAIRLALELCRQSTDLEECLRLLKTAIKALENALRHPDSTTAKARLMAITARLL |
| | AQQLRTQHPDSQAARDAEKLADQAERAVRLATRLYEEHPNAEISEMCSQAAYAAALMASIAA |
| | ILAQRHPDSQIARDLIRLASELAEMVKRMCER (SEQ ID NO: 35) |
| | (M)GTREESLKEQLRSLREQAELAARLLRLQREGSSDEDVKELVAEIIKLIMEQLLLIAEQL |
| | LGRSEAAELALRAIRLALELCRQSTDLEECLRLLKTAIKALENALRHPDSTTAKARLMAITA |
| | RLLAQQLRTQHPDSQAARDAEKLADQAERAVRLATRLYEEHPNAEISEMCSQAAYAAALMAS |
| | IAAILAQRHPDSQIARDLIRLASELAEMVKRMCER(GGSWGLEHHHHHH) (SEQ ID |
| | NO: 36) |
| >D2_Wm- | GTREELAKELLRSLREQAESLARQLRLQREGSSDEDVKELAAEQIKLIMEQLLLIAELTLGR |
| 01_truncB | SEAAELALDAIRQALEACRTMDNQEACTRLLKLAIQMLELATRAPDAEAAKLALEAAKKAIE |
| | LANRHPGSQAAEDATKLAQQAMEAVRLALKLYEEHPNADIADLCRRAAAEAAEAASKAAELA |
| | QRHPDSQAARDAIKLASQAAEAVKLACELAQEHPNADKAKLCILLASAAALLASIAAMLAQR |
| | HPDSQEARDMIRIASELAELVKEICER (SEQ ID NO: 37) |
| | (M)GTREELAKELLRSLREQAESLARQLRLQREGSSDEDVKELAAEQIKLIMEQLLLIAELT |
| | LGRSEAAELALDAIRQALEACRTMDNQEACTRLLKLAIQMLELATRAPDAEAAKLALEAAKK |
| | AIELANRHPGSQAAEDATKLAQQAMEAVRLALKLYEEHPNADIADLCRRAAAEAAEAASKAA |
| | ELAQRHPDSQAARDAIKLASQAAEAVKLACELAQEHPNADKAKLCILLASAAALLASIAAML |
| | AQRHPDSQEARDMIRIASELAELVKEICER (SEQ ID NO: 38) |
| Point Group | |
| nanocage | |
| >T_Wm-1606 | GDEEKKKELLKQLEDSLIELIRILAELKEMLERLEKNPDKDTIVKVLKVIVKAIEASVANQA |
| | ISAMNQGADANAKDSDGRTPLHHAAEAGAAAVVKVAIDAGADVNEKDSDGRTPLHHAAENGH |
| | AEVVTLLIEKGADVNEKDSDGRTPLHHAAENGHDEVVLILLLKGADVNAKDSDGRIPLHHAA |
| | ENGHKRVVLVLILAGADVNTSDSDGRTPLDLAREHGNEEVVKALEKQ (SEQ ID NO: 39) |
| | (M)GDEEKKKELLKQLEDSLIELIRILAELKEMLERLEKNPDKDTIVKVLKVIVKAIEASVA |
| | NQAISAMNQGADANAKDSDGRTPLHHAAEAGAAAVVKVAIDAGADVNEKDSDGRTPLHHAAE |
| | NGHAEVVTLLIEKGADVNEKDSDGRIPLHHAAENGHDEVVLILLLKGADVNAKDSDGRIPLH |
| | HAAENGHKRVVLVLILAGADVNTSDSDGRIPLDLAREHGNEEVVKALEKQ(GGWLEHHHHHH) |
| | (SEQ ID NO: 40) |
| >I32_Wm-42A | GGSELEIVIRLQILNLELARKLLEAVARLQELNIDLVRKTSELTDEKTIREEIRKVKEESKR |
| | IVKEAEDEIKKAALISADLAAKAIKRAIDRAKKLLEKGEKEDAEDVLREARSAIRLVTELLE |
| | RIAKNSSTPEEALRAAELLVRLIILLIKIAALLAAAGNKEEADKVLDEAKELIERVRELLEK |
| | ISKNSDTPELSKRAKELELILRLADLAIKAMKNTGSDEARQAVKEMARLAKEALEMGMSEAA |
| | KAAIELLELLAEAFAGSDVASLAVKAIAKIAETALRNGS (SEQ ID NO: 41) |
| | (M)GGSELEIVIRLQILNLELARKLLEAVARLQELNIDLVRKTSELTDEKTIREEIRKVKEE |
| | SKRIVKEAEDEIKKAALISADLAAKAIKRAIDRAKKLLEKGEKEDAEDVLREARSAIRLVTE |
| | LLERIAKNSSTPEEALRAAELLVRLIILLIKIAALLAAAGNKEEADKVLDEAKELIERVREL |
| | LEKISKNSDTPELSKRAKELELILRLADLAIKAMKNTGSDEARQAVKEMARLAKEALEMGMS |
| | EAAKAAIELLELLAEAFAGSDVASLAVKAIAKIAETALRNGS (SEQ ID NO: 42) |
| | *the bolded Ser residue may, for example, be modified to a Cys |
| | residue |
| >I32_Wm-42B | GSDTAKEAIQRLEDLARKYSGSDVASLAVKAIEKIARTAVENGSEETAEEAEKRLRELAEDY |
| | QGSNVASLAASAIAEIAAARARFAAREMGDPRVEEIAKELERLAKEAAERVERRPDSEEDYR |
| | KLELAALIIKLFVSLLKQKRLAERLKELLRELERLQREGSSDEDVRELLREIKELVEEIEKL |
| | ARKQEYLVTELAKMM (SEQ ID NO: 43) |
| | (M)GSDTAKEAIQRLEDLARKYSGSDVASLAVKAIEKIARTAVENGSEETAEEAEKRLRELA |
| | EDYQGSNVASLAASAIAEIAAARARFAAREMGDPRVEEIAKELERLAKEAAERVERRPDSEE |
| | DYRKLELAALIIKLFVSLLKQKRLAERLKELLRELERLQREGSSDEDVRELLREIKELVEEI |
| | EKLARKQEYLVTELAKMM(GGSGGSGGSGGSLEHHHHHH) (SEQ ID NO: 44) |
| | *the bolded Ser residue may, for example, be modified to a Cys |
| | residue |
| >C3_HF_Wm_ | GKELEIVARLQQLNIELARKLLEAVARLQELNIDLVRKTSELTDEKTIREEIRKVKEESKRI |
| 0024A | VEEAEQEIRKAEAESLRLTAEAAADAARKAALRMGDERVRRLAAELVRLAQEAAEEATRDPN |
| | SSDQNEALRLIILAIEAAVRALDKAIEKGDPEDRERAREMVRAAVRAAELVQRYPSASAANE |
| | ALKALVAAIDEGDKDAARCAEELVEQAEEALRKKNPEEARAVYEAARDVLEALQRLEEAKRR |
| | GDEEERREAEERLRQACERARKKN (SEQ ID NO: 45) |
| | (M)GKELEIVARLQQLNIELARKLLEAVARLQELNIDLVRKTSELTDEKTIREEIRKVKEES |
| | KRIVEEAEQEIRKAEAESLRLTAEAAADAARKAALRMGDERVRRLAAELVRLAQEAAEEATR |
| | DPNSSDQNEALRLIILAIEAAVRALDKAIEKGDPEDRERAREMVRAAVRAAELVQRYPSASA |
| | ANEALKALVAAIDEGDKDAARCAEELVEQAEEALRKKNPEEARAVYEAARDVLEALQRLEEA |
| | KRRGDEEERREAEERLRQACERARKKN(GGSLEHHHHHH) (SEQ ID NO: 46) |

The polypeptides of the disclosure can be used, for example to prepare multi-scale protein complexes by combinatorial assembly of oligomeric helical bundle and repeat protein building blocks. As disclosed in the example, the inventors have developed of methods for creating large and modular libraries of building blocks by fusing modified helical repeat proteins to parametric helical bundles, exemplified by the polypeptides disclosed herein. These polypeptides can then be used to generate symmetric assemblies, as exemplified by the oligomers disclosed herein.

As disclosed in the examples, the polypeptides may have substantial sequence variability while retaining their structures. In one embodiment, amino acid changes from the reference polypeptide of any one of SEQ ID NOS:1-46 are conservative amino acid substitutions. As used here, “conservative amino acid substitution” means that:
hydrophobic amino acids (Ala, Cys, Gly, Pro, Met, Val, Ile, Leu) can only be substituted with other hydrophobic amino acids;
hydrophobic amino acids with bulky side chains (Phe, Tyr, Trp) can only be substituted with other hydrophobic amino acids with bulky side chains;
amino acids with positively charged side chains (Arg, His, Lys) can only be substituted with other amino acids with positively charged side chains;
amino acids with negatively charged side chains (Asp, Glu) can only be substituted with other amino acids with negatively charged side chains; and
amino acids with polar uncharged side chains (Ser, Thr, Asn, Gln) can only be substituted with other amino acids with polar uncharged side chains.

The amino acid sequences of the polypeptides in Table 1 include highlighted amino acid residues. In one embodiment, at least 1, 2, 3, 4, 5, 6, 7, 8, 9, 10, 11, 12, 13, 14, 15, 16, 17, 18, 19, 20, 21, 22, 23, 24, 25, or more, or all of the non-polar residues in bold font are invariant relative to the reference polypeptide. These residues may serve to stabilize the structure of the polypeptide. As will be understood by those of skill reviewing Table 1, not all of the bold-font residues are non-polar amino acids. As used herein, “non-polar residues” are Ala, Cys, Gly, Pro, Met, Val, Ile, Leu, Phe, Tyr, Trp. In another embodiment, at least 1, 2, 3, 4, 5, 6, 7, 8, 9, 10, 11, 12, 13, 14, 15, 16, 17, 18, 19, 20, 21, 22, 23, 24, 25, or more, or all of the residues in bold font (including polar residues in bold font) as shown in Table 1 are invariant relative to the reference polypeptide.

In all polypeptide embodiments for all aspects of the disclosure, in one embodiment the optional residues are present in the polypeptides and considered in determining percent identity relative to the reference sequence; in other embodiments, the optional residues are not present and are not considered in determining percent identity relative to the reference sequence.

The polypeptides may comprise any further functional domain fused to the polypeptide that may be of use for an intended purpose of oligomers comprising the polypeptides. In various non-limiting embodiments, the resulting fusion protein comprises an additional functional domain such as detectable proteins, purification tags, protein antigens, and protein therapeutics.

In another aspect, the present disclosure provides nucleic acids, including isolated nucleic acids, encoding the polypeptides or fusion protein of any embodiment or combination of embodiments of the present disclosure. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, secretory signals, nuclear localization signals, and plasma membrane localization signals. It will be apparent to those of skill in the art, based on the teachings herein, what nucleic acid sequences will encode the polypeptides and fusion proteins of the invention.

In a further aspect, the present disclosure provides expression vectors comprising the nucleic acid of any embodiment of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector.

In one aspect, the present disclosure provides host cells that comprise the polypeptides, oligomers, expression vectors and/or nucleic acids disclosed herein, wherein the host cells can be either prokaryotic or eukaryotic. The cells can, for example, be transiently or stably engineered to incorporate the polypeptides, oligomers, expression vectors and/or nucleic acids of the disclosure, using standard techniques. A method of producing a polypeptide according to the invention is an additional part of the disclosure. The method comprises the steps of (a) culturing a host according to this aspect of the disclosure under conditions conducive to the expression of the polypeptide, and (b) optionally, recovering the expressed polypeptide.

In another aspect, the disclosure provides oligomers, comprising two or more polypeptides or fusion proteins disclosed herein. As disclosed in the examples, the inventors have used the polypeptides of the disclosure to generate a broad range of symmetric assemblies.

In one embodiment, the oligomer comprises a homo-oligomer. In exemplary such embodiments, the homo-oligomer comprises two or more identical polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-26, 39-40, and 45-46.

In another embodiment, the oligomer comprises a hetero-oligomer. In exemplary embodiments, the hetero-oligomer comprises two different polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of:

SEQ ID NO: 27 and 29;

SEQ ID NO:27 and 30;

SEQ ID NO:28 and 29;

SEQ ID NO:28 and 30;

SEQ ID NO: 31 and 33;

SEQ ID NO:31 and 34;

SEQ ID NO:32 and 33;

SEQ ID NO:32 and 34;

SEQ ID NO: 35 and 37;

SEQ ID NO:35 and 38;

SEQ ID NO:36 and 37;

SEQ ID NO:36 and 38;

SEQ ID NO: 41 and 43;

SEQ ID NO:41 and 44;

SEQ ID NO:42 and 43; and

SEQ ID NO:42 and 44.

In a further embodiment, the oligomer comprises a two-component dihedral assembly. In exemplary embodiments, the two component dihedral assembly comprises two different polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of:

SEQ ID NO: 27 and 29;

SEQ ID NO:27 and 30;

SEQ ID NO:28 and 29;

SEQ ID NO:28 and 30;

SEQ ID NO: 31 and 33;

SEQ ID NO:31 and 34;

SEQ ID NO:32 and 33;

SEQ ID NO:32 and 34;

SEQ ID NO: 35 and 37;

SEQ ID NO:35 and 38;

SEQ ID NO:36 and 37; and

SEQ ID NO:36 and 38.

In a further embodiment, the oligomer comprises a one-component tetrahedral protein cage. In exemplary embodiments, the one-component tetrahedral protein cage comprises the polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:39 or 40.

In one embodiment, the oligomer comprises a two-component icosahedral protein cage. In exemplary embodiments, the two icosahedral protein cage comprises two different polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of:

SEQ ID NO: 41 and 43;

SEQ ID NO:41 and 44;

SEQ ID NO:42 and 43; and

SEQ ID NO:42 and 44.

The oligomers may be used for any suitable purpose. In some embodiments, the polypeptides that make up the oligomers may include polypeptides fused to an immunogen, and the resulting oligomers may be used as a vaccine. In other embodiments, the oligomers may include polypeptides fused to a polypeptide therapeutic, and the resulting oligomers may be used for delivery of the therapeutic. In other embodiments, the oligomers may be used for biomaterial production.

In another aspect, the disclosure provides method for designing multi-scale protein complexes by combinatorial assembly of oligomeric helical bundle and repeat protein building blocks, comprising any methods as described in the examples that follow.

### EXAMPLES

### Hierarchical Design of Multi-Scale Protein Complexes by Combinatorial Assembly of Oligomeric Helical Bundle and Repeat Protein Building Blocks

A goal of de novo protein design is to develop a systematic and robust approach to generating complex nanomaterials from stable building blocks. Due to their structural regularity and simplicity, a wide range of monomeric repeat proteins and oligomeric helical bundle structures have been designed and characterized. Here we describe a stepwise hierarchical approach to building up multi-component symmetric protein assemblies using these structures. We first connect designed helical repeat proteins (DHRs) to designed helical bundle proteins (HBs) to generate a large library of heterodimeric and homooligomeric building blocks; the latter have cyclic symmetries ranging from C2 to C6. All of the building blocks have repeat proteins with accessible termini, which we take advantage of in a second round of architecture guided rigid helical fusion (WORMS) to generate larger symmetric assemblies including C3 and C5 cyclic and D2 dihedral rings, a tetrahedral cage, and a 120 subunit icosahedral cage. Characterization of the structures by small angle x-ray scattering, x-ray crystallography, and cryo-electron microscopy demonstrates that the hierarchical design approach can accurately and robustly generate a wide range of macromolecular assemblies; with a diameter of 43 nm, the icosahedral nanocage is the largest structurally validated designed cage to date. The computational methods and building block sets described here provide a very general route to new de novo designed symmetric protein nanomaterials.

Computational protein design has been used to create proteins that self-assemble into a wide variety of higher order structures. However, interface design remains challenging, and designable interface quality is heavily dependent on how well the building blocks complement each other during design. An alternative approach which avoids the need for designing new interfaces is to fuse oligomeric protein building blocks with helical linkers; however, lack of rigidity has made the structures of these assemblies difficult to precisely specify. More rigid junctions created by overlapping ideal helices and designing around the junction region has its own set of challenges in comparison to designing a new non-covalent protein-protein interface: first, for any pair of protein building blocks, there are far fewer positions for rigid fusion than are for unconstrained protein-protein docking limiting the space of possible solutions, and second, while in the non-covalent protein interface case the space searched can be limited by restricting building blocks to the symmetry axes of the desired nanomaterial, this is not possible in the case of rigid fusions, making the search more difficult as the number of building blocks increases.

A potential solution to the issue of having smaller numbers of possible fusion positions for a given pair of building blocks in the rigid helix fusion method is to systematically generate large numbers of building blocks having properties ideal for helix fusion. Attractive candidates for such an approach are de novo helical repeat proteins (DHRs) consisting of a tandemly repeated structural unit, which provide a wide range of struts of different shape and curvature for building nanomaterials, and parametric helical bundles (HBs) which provide a wide range of preformed protein-protein interfaces for locking together different protein subunits in a designed nanomaterial. Many examples of both classes of designed proteins have been solved by x-ray crystallography, and they are typically very stable. We reasoned that by systematically fusing DHR “arms” to central HB “hubs” we could generate building blocks with a wide range of geometries and valencies that, because of the modular nature of repeat proteins, enable a very large number of rigid helix fusions: given two such building blocks with N- and C-terminally extending repeat protein arms, the potentially rigid fusion sites are any pair of internal helical residues in the DHR arms.

With a large library of building blocks, the challenge is then to develop a method to very quickly traverse all possible combinations of fusion locations. We present here WORMS, a software package that uses geometric hashing of transforms to very quickly and systematically identify the fusion positions in large sets of building blocks that generate any specified symmetric architecture, and describe the use of the software to design a broad range of symmetric assemblies.

### Results

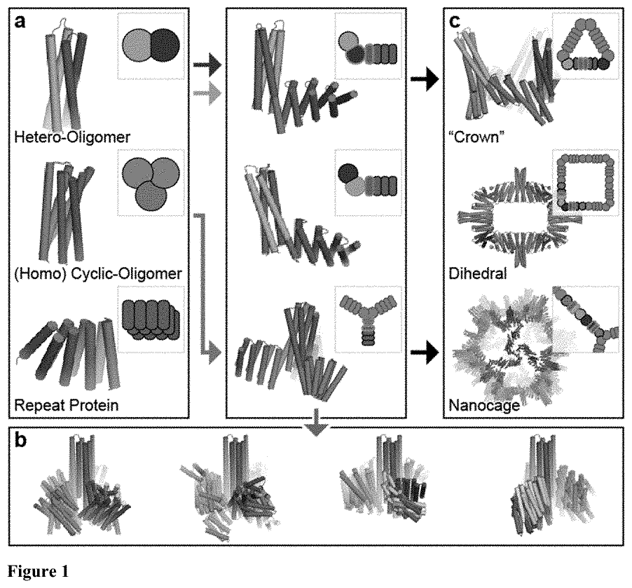

We describe the development of methods for creating large and modular libraries of building blocks by fusing DHRs to HBs, and then using them to generate symmetric assemblies by rapidly scanning through the combinatorially large number of possible rigid helix fusions for those generating the desired architecture. We present the new methodology and results in two sections. In section one, we describe the systematic generation of homo- and hetero-oligomeric building blocks from de novo designed helical bundles, helical oligomers, and repeat proteins (FIG. 1a). In the second section, we describe the use of these building blocks to assemble a wide variety of higher order symmetric architectures (FIG. 1c).

### Section 1: Systematic Generation of Oligomeric Building Blocks

To generate a wide variety of building blocks, we explored two different methodologies for fusing DHRs to HBs (FIG. 1a). The first is to dock the DHR units to the HBs, redesign the residues at the newly created interface, and then build loops between nearby termini (HelixDock, HD). The second protocol simplifies the process by overlapping the helical termini of the DHRs and HBs and designing only the immediate residues around the junction (HelixFuse, HF). As an example of the combinatorial diversity that can be generated due to the large number of possible internal helical fusion sites in a DHR (nearly all helical residues), a single terminus from a single helical bundle (2L6HC3-12²⁰, N-terminus) combined with the library of 44 verified DHRs resulted in 259 different structures (FIG. 1b).

HelixDock (HD) approach: 44 DHRs with validated structures²³ and 11 HBs^{20,24} (including some without pre-verified structures) were selected as input scaffolds for symmetrical docking using a modified version of the SICDOCK™ software³. In each case, N copies of the DHR, one for each monomer in the helical bundle, were symmetrically docked onto the HB, sampling all six degrees of freedom, to generate star shaped structures with repeat protein arms emanating symmetrically from the helical bundle in the center. Docked configurations with linkable N- and C-termini within a distance cutoff of 9 Å with interfaces predicted to yield low energy designs²⁵ were then subjected to Rosetta™ sequence design to optimize the residue identity and packing at the newly formed interface. Designs with high predicted domain-domain binding energy and shape complementarity²⁶ were identified, and loops connecting chain the termini were built using the ConnectChainsMover¹⁷. Structures with good loop geometry (passing worse9merFilter and FoldabilityFilter) were forward folded with Rosetta™ Remodel™²⁷ symmetrically, and those with sequences which fold into the designed structure in silico were identified.

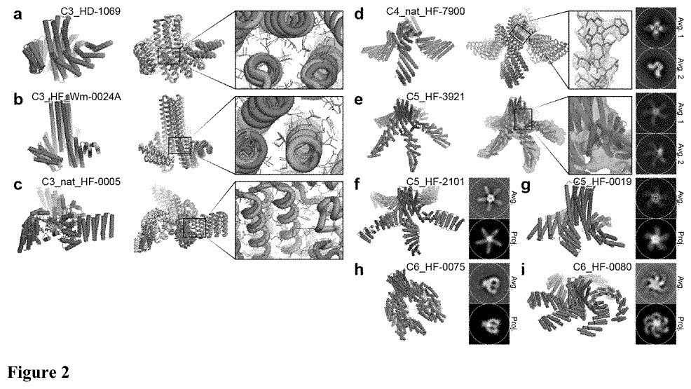

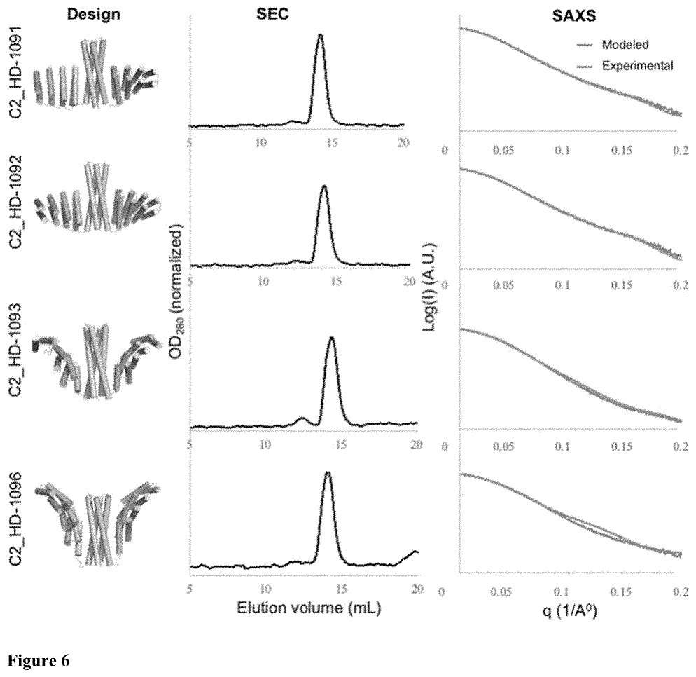

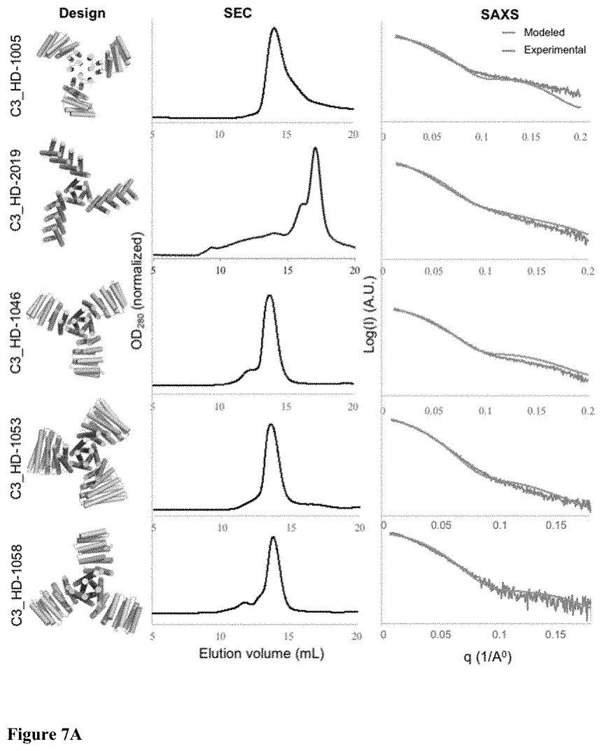

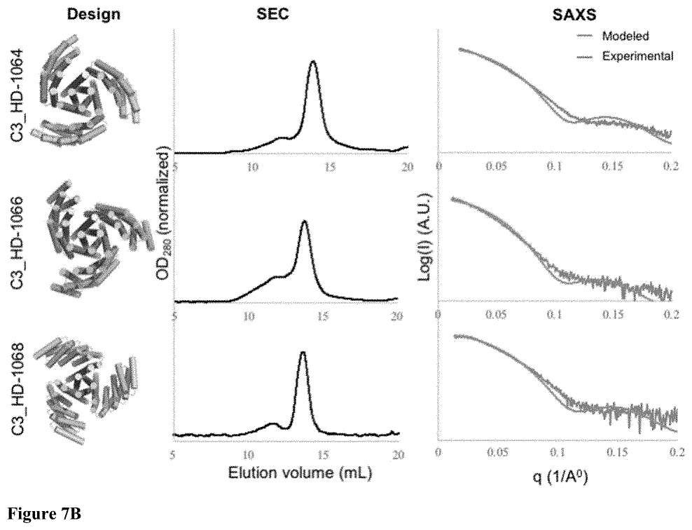

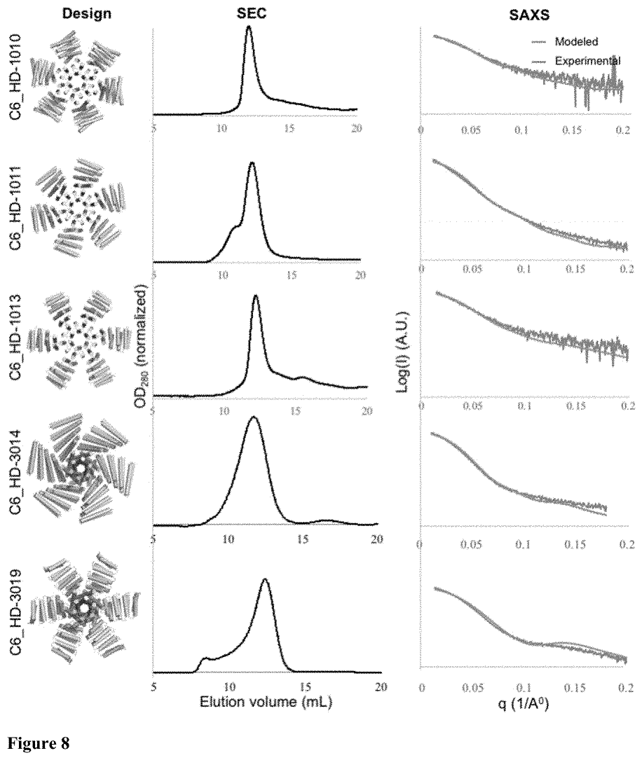

Synthetic genes encoding a subset of the selected designs with a wide range of shapes were synthesized and the proteins expressed in E. coli. Of the 115 sequences ordered successfully synthesized, 65 resulted in soluble protein. Those with poor expression and/or solution behavior were discarded. Of the remaining, 39 had relatively monodisperse Size Exclusion Chromatography (SEC) profiles that matched what was expected from the design. Of the ones selected for small angle X-ray scattering (SAXS), 17 had profiles close to those computed from the design models (FIG. 6-8). Design C3_HD-1069, was crystallized and solved to 2.4 Å (FIG. 2a). Although the two loops connecting to the HB are unresolved in the structure, the resulting placement of the DHR remains correct (unresolved loops were also present in the original HB structure (2L6HC3_6)²⁰. The resolved rotamers at the newly designed interface between the HB and DHR are also as designed.

HelixFuse (HF) approach: The same set of DHRs and HBs were combinatorially fused together by overlapping the terminal helix residues in both directions (“AB”: c-terminus of HB to n-terminus of DHR, “BA”: n-terminus of HB to c-terminus of DHR)¹⁷. On the HB end, up to 4 residues were allowed to be deleted to maximize the sampling space of the fusion while maintaining the structural integrity of the oligomeric interface. On the DHR end, deletions up to a single repeat were allowed. After the C-beta atoms are superimposed, a RMSD check across 9 residues was performed to ensure that the fusion results in a continuous helix. If no residues in the fused structure clash (Rosetta™ centroid energy<10), sequence design was carried out at all positions within 8 Å of the junction. This first step of the fusion sampling is wrapped into the Rosetta™ MergePDBMover¹⁷. After sequence design around the junction region^{14,28}, fusions were then evaluated based on the number of helices interacting across the interface (at least 3), buried surface (sasa>800) across the junction, and shape complementarity (sc>0.6) to identify designs likely to be rigid across the junction point. In total, the building block library generated in silico by HelixFuse using HB hubs and DHR arms in this set consists of 490 C2s, 1255 C3s, 107 C5s, and 87 C6s.

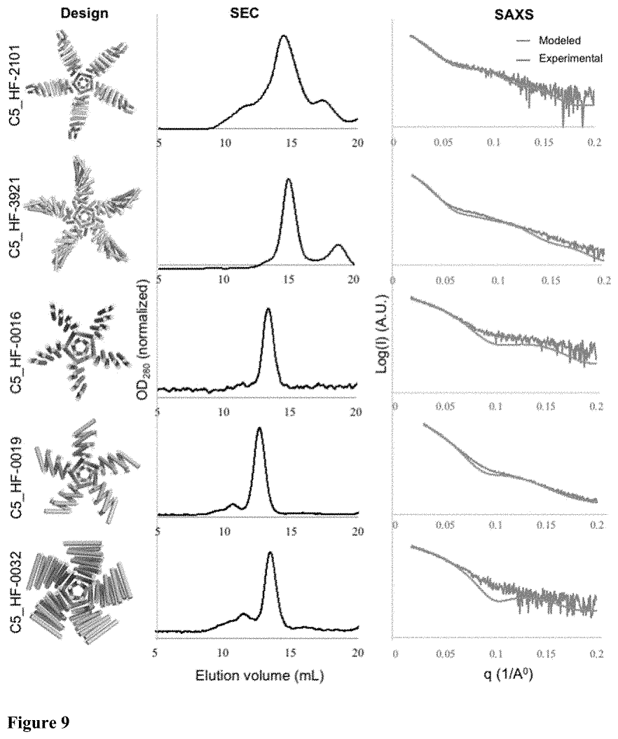

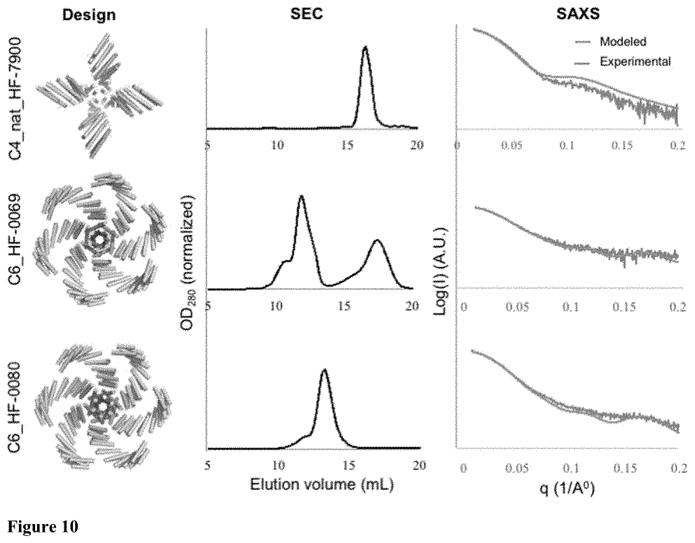

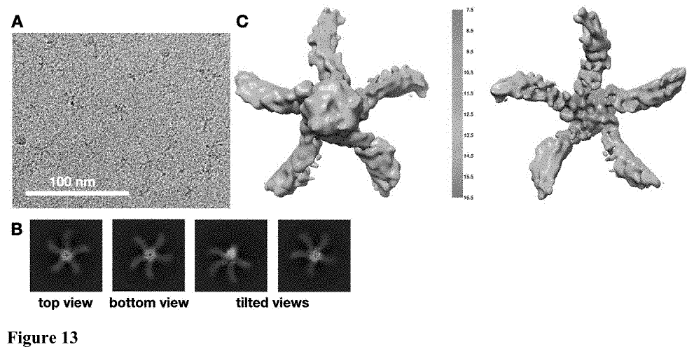

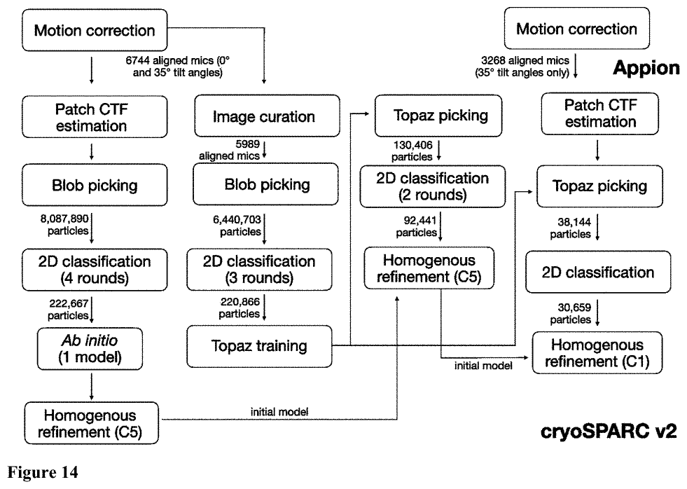

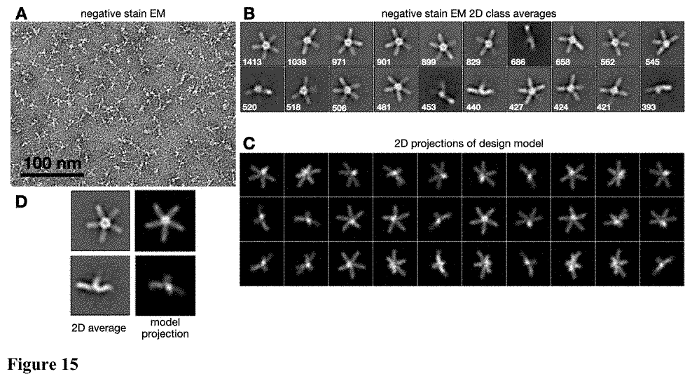

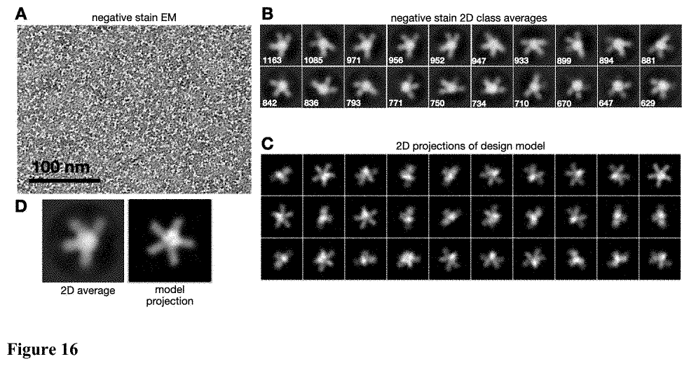

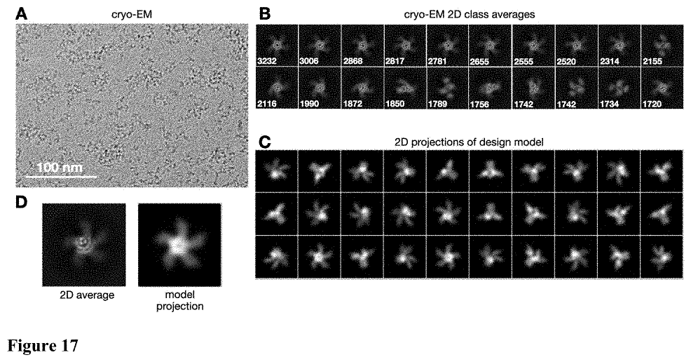

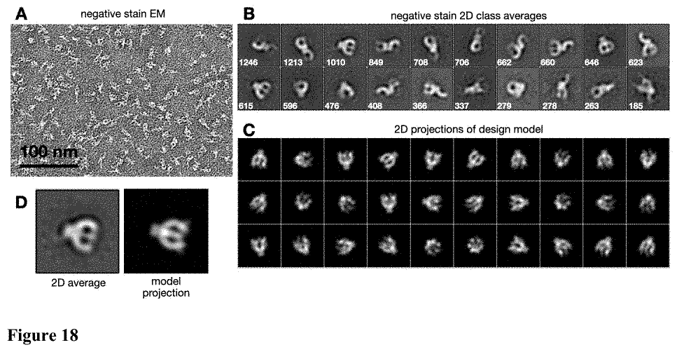

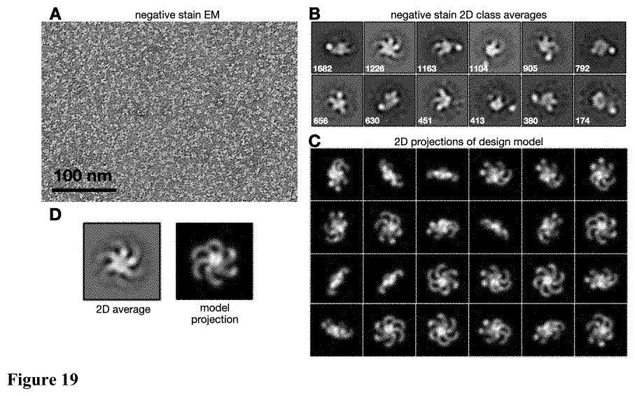

As a proof of concept, select fusions to C5 (5H2LD-10⁷) and C6 (6H2LD-8) (in press) helical bundles were tested experimentally, as structures of higher cyclic symmetries were historically more difficult to design thus resulting in a lack of available scaffolds. Contrarily, larger structures are easier to experimentally characterize via electron microscopy due to their size. A total of 65 designs whose genes encoding the designs were synthesized and subsequently expressed in E. coli, 45 were soluble, and 23 were monodisperse by SEC. Of the ones that were selected for SAXS analysis, 7 had matching SAXS profiles (FIG. 9-10). Cryo-electron microscopy of C5_HF-3921 followed by 3D reconstruction showed that the positions of the helical arms are close to the design model (FIG. 2e, FIG. 13-14). By negative-stain electron microscopy (EM), C5_HF-2101, C5_HF-0019, C6_HF-0075, and C6_HFuse-0080 (FIG. 2f-i respectively) were class averaged and the top-down view clearly resembles that of the designed model and its predicted projection map (FIGS. 15, and 17-19 respectively). From negative-stain EM class averaging, off-target states can sometimes be observed; most obvious in C5_HF-0007 (FIG. 16) and C6_HF-0075 (FIG. 18), and less in C5_HF-0019 (FIG. 17), where in some cases an incorrect number of DHR arms can be observed in the 2D class averages.

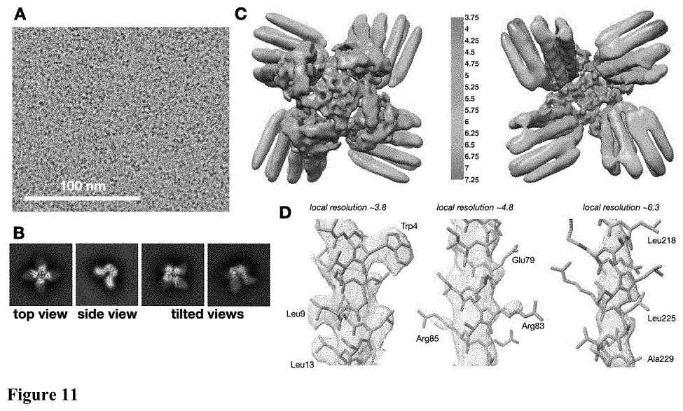

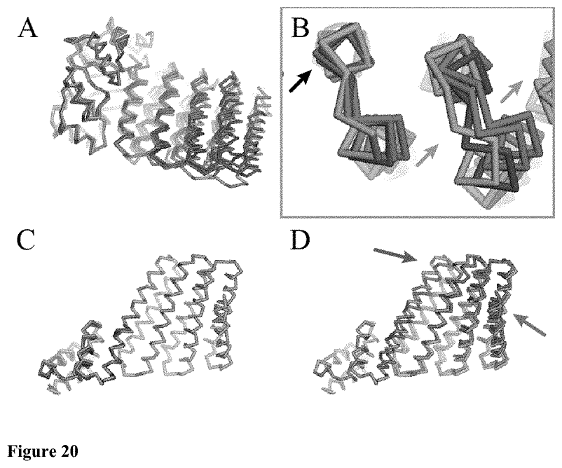

We also applied the method to two non-helical bundle oligomers—1wa3, a native homo-trimer²⁹ and tpr1C4_pm3, a designed homo-tetramer²⁵. As described above, we fused DHRs to the N-terminal helix of 1wa3 and the C-terminal helix of tpr1C4_pm3. For 1wa3, from the 13 designs were expressed for experimental validation, 10 displayed soluble expression and showed clean monodispersed peaks by SEC. Through X-ray crystallography, we were able to solve C3_nat_HF-0005 to 3.32 Å resolution (FIG. 2c). A total of 16 tpr1C4_pm3 fusions were tested, 14 found to be soluble, and 10 displayed monodispersed peaks by SEC. The best behaving designs were analyzed by electron microscopy. C4_nat_HF-7900 was found to form monodisperse particles by cryo EM, with the 3D reconstruction modeled to 3.7 Å resolution (FIG. 2d, FIG. 10-12). Both the crystal structure of C3_nat_HF-0005 and the model of the cryo-EM reconstruction of C4_nat_HF-7900 show very good matches near the oligomeric hub of the protein where side chains are clearly resolved and as expected. However, it can be seen that they deviate from the design model at the most distal portions of the structure. This is likely due to the inherent flexibility of the unsupported terminal helices of the DHRs^{17,23,30} and lever arm effects which increase with increasing distance from the fusion site (FIG. 20).

To extend the complexity of structures that can be generated, we built libraries of heteromeric two chain building block by fusing repeat proteins to two hetero-dimeric helical bundles (DHD-13, DHD-37)²¹ (FIG. 1a). The fusion steps are identical, except for an additional step of merging the chain A and chain B fusions and checking for clashes and incompatible residues. In total, 2740 heterodimers were generated in silico to be part of the library. While the homo-oligomeric fusions are good building blocks for objects with higher order point group symmetries, hetero-oligomeric fusions are needed at segments without symmetry, such as building cyclic structures and/or connecting different axes of symmetry in higher order architectures (described below).

With a sufficiently high design success rate, the individual oligomers do not need to be experimentally verified before being used to build larger structures. Since all building blocks terminate in repeat proteins which can be fused anywhere along their length, the total number of possible three building block fusions which can be built from this set is extremely large, which could offset the degree of freedoms lost to symmetry constraints. The combined library for higher order oligomers consists of both HelixDock and HelixFuse generated building blocks; overall, the HelixFuse structures tended to have smaller interfaces across the junction, and thus less overall hydrophobicity than those generated by HelixDock. While the HelixFuse are less globular than their HelixDock counterparts, the smaller interface may contribute to the higher fraction of designs being soluble (˜70% vs ˜55%). The HelixDock method also requires an additional step of building a new loop between the HB and DHR, which is another potential source of modeling error, and takes significantly more computational hours. Overall, the final fraction with single dominant species in SEC traces (examples shown in FIG. 6-10) profiles are similar (˜35%).

Section II: Assembly of Higher Order Symmetric Structures from Repeat Protein-Helical Bundle Fusion Building Blocks

To generate a wide range of novel protein assemblies without interface design, we took advantage of the protein interfaces in the library of building blocks described in the previous section, which are oligomers with repeat protein arms. Assemblies are formed by splicing together alpha helices of the repeat protein arms in different building blocks. In our implementation, the user specifies a desired architecture and the symmetries and connectivity of the constituent building blocks. The method then iterates through splices of all pairs of building blocks at all pairs of (user specified, see methods) helical positions; this very large set is filtered on the fly based on the rms of the spliced helices, a clash check, off-architecture angle tolerance, residue contact counts around splice, helix contact count, and redundancy; all of which can be user specified parameters (see methods). The rigid body transform associated with each splice passing the above criteria is computed; for typical pairs of building blocks allowing 100 residues, 100×100=10,000 unfiltered splices are possible.

Assemblies of these building blocks are modeled as chains of rigid bodies, using the transform between coordinate frames of entry and exit splices, as well as transform between entry splice and coordinate frames of the building blocks. Assemblies are built, in enumerative fashion or with monte carlo, by simple matrix multiplication. For efficiency, only prefiltered splices are used. This technique allows billions of potential assemblies to be generated per cpu hour. Criteria for a given assembly design problem can include any operation defined on the rigid body positions of the building blocks. In this work, we use the transform from the start and end building blocks. To form Cx cyclic oligomers, the rotation angle of the transform must be 360/x, and the translation along the rotation axis must be zero. To form tetrahedral, octahedral, icosahedral, and dihedral point group symmetries from cyclic building blocks, the symmetry axes of the start and end building blocks must intersect, and form the appropriate angle for the desired point group; for example, a 900 angle creates dihedral symmetry.

This rapid symmetric architecture assembler through building block fusion has been implemented in a program called WORMS (Wm) which provides users with considerable control over building block sets, geometric tolerances, and other parameters and enables rapid generation of a wide range of macromolecular assemblies. The desired architecture is entered as a config file (or command line option) in the following format illustrated for a 3-part fusion with icosahedral symmetry:

[‘C3 N’,orient(None,‘N’)),(‘Het:CN’,orient(‘C’,‘N’)),(‘C2_C’,orient(‘C’,None)]

Icosahedral(c3=0,c2=−1)

The architecture is specified first, here an icosahedral structure constructed from a C3 and a C2 building block, and then how the selected building blocks types from the loaded databases are to be linked together (like a worm). In this example, a C3 building block with an available N-terminus ‘C3_N’ is to be fused to a hetero-dimeric building block ‘Het:CN’ via an available C-terminus, and the N-terminus of the same ‘Het:CN’ is in turn to be fused to a third C2 building block ‘C2_C’ through an available C-terminus. The ‘None’ designation marks that there are no additional unique connections to be made on that segment. Through the assignment of ‘c3=0’ and ‘c2=−1’, the first and last building blocks are declared as the C3 component and the C2 component, respectively. The building blocks are cached the first time they are read in from the database files, which can range from a single entry per type to thousands, due to the combinatorial nature of the first fusion step. See supplementary information for more details regarding additional options, architecture definitions, and database syntax. With hundreds to thousands of building blocks each with ˜100 residues available for fusion, the total number of three way fusions is on the order of greater than 10¹⁴, so optimization of efficiency in both memory usage and CPU requirements was critical in WORMS software development.

Once building block combinations are identified that generate the designed architecture (within a user specifiable tolerance), explicit atomic coordinates are calculated and used for clash checking, redundancy filtering, and any other filtering that requires atomic coordinates. Models for each assembly passing user specified tolerances are constructed in Rosetta™, scored and output for subsequent sequence design.

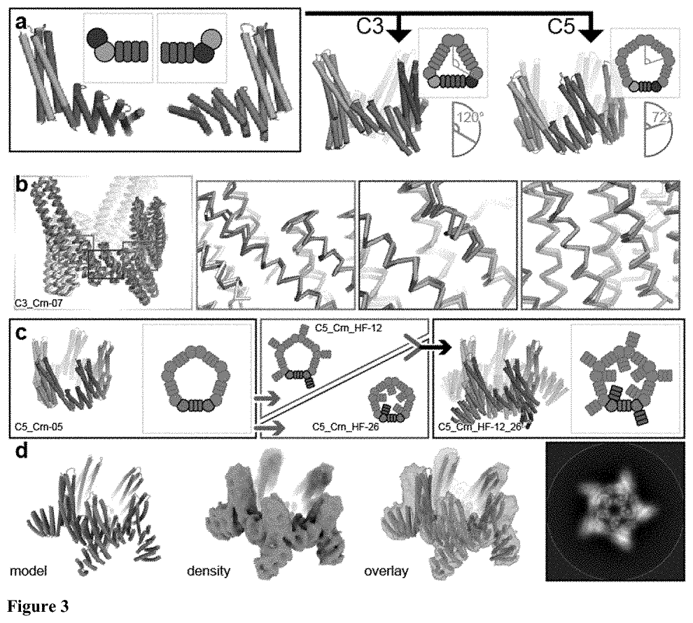

Generation of cyclic “crowns” (Crn): We generated C3, C4, and C5 assemblies with WORMS using two designed heterodimer fusions from HelixFuse, as described above. This resulted in head-to-tail cyclic ring structures (FIG. 3a), generated by the following configuration (C3 as an example):

[(‘Het:CN’,orient(None,‘N’)),(‘Het:CN’,orient(‘C’,None))]

Cyclic (3)

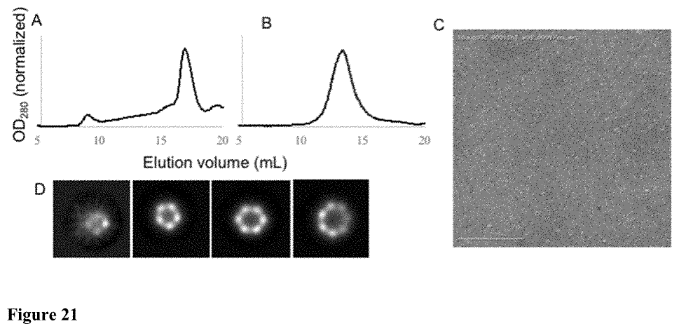

Following fusion, the junction residues were redesigned to favor the fusion geometry and filtered as above. Seven C3s, seven C4s, and eight C5s were selected and tested experimentally. All yielded soluble protein, and 6, 2, and 1 respectively showed a single peak at the expected elution volume via SEC. We solved the structure of the C3_Cm-05 to 3.19A resolution (FIG. 3b). The overall topology is as designed and the backbone geometry at each of the three junctions is close to the design model. A deviation at the tip of the undesigned heterodimeric HB is likely to due to crystal packing. C5_Crn-07 chromatographed as a single peak by SEC and was found to be predominantly C5 by negative-stain EM (FIG. 3d), but minor off-target species (C4, C6, and C7) were also observed (FIG. 21). Each of these structures experimentally verifies three distinct helical fusions (two HelixFuse, one WORMS) from a previously unverified building block library.

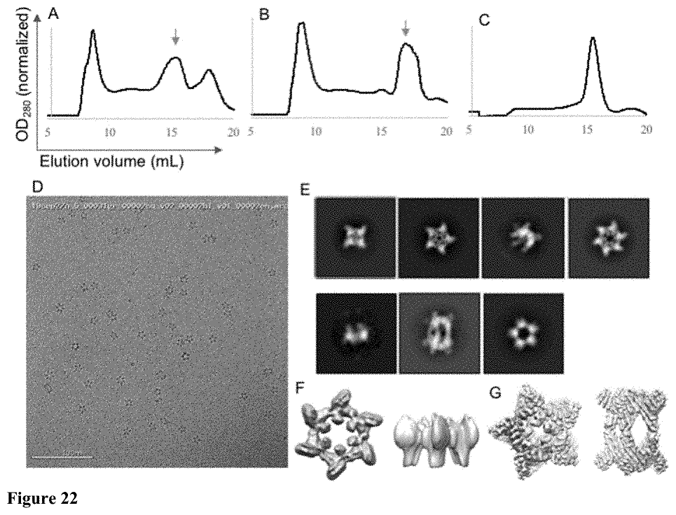

To further increase the diversity of the crown structures, we recursively ran HelixFuse on both termini of C5_Crn-07 (FIG. 3c). Six (6) N-terminal and 24 C-terminal fusions were selected and experimentally tested. All were soluble, but had large soluble aggregate fractions when analyzed by SEC. When the peaks around the expected elution volumes were analyzed by negative-stain EM, ring-like structures were found in many of the samples. To facilitate EM structure determination, we combined a c-terminal fusion (C5_Crn_HF-12) and an n-terminal (C5_Crn_HF-26) fusion to generate C5_Crn_HF-12_26 (FIG. 3c), which resulted in a much cleaner and monodisperse SEC profile (FIG. 22). Cryo-electron microscopy of 1226 revealed the major population of C5 (77%) structures in addition to C4 (1%), D5 (8%), and C6 (12%) subpopulations (FIG. 22). We hypothesize that the D5 structure is due to transient interactions of histidines placed on the loops for protein purification. The final 3D reconstruction to 5.6 Å resolution shows that the major characteristics of the design model are present, despite some splaying of the undesigned portion of the heterodimeric HB relative to the design model (FIG. 3d).

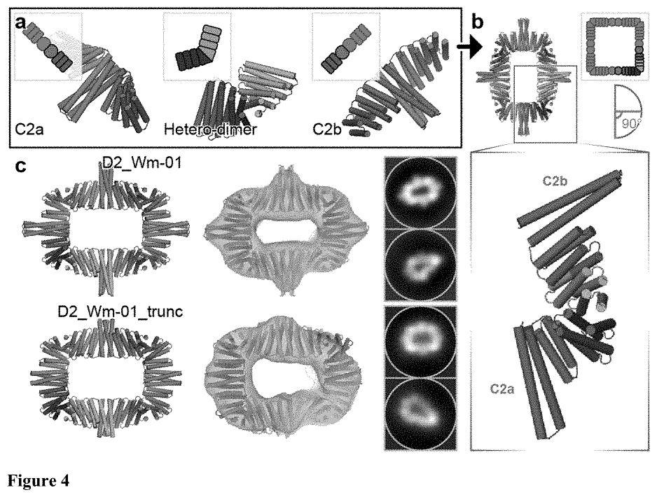

Generation of two-component dihedral assemblies: Dihedral symmetry protein complexes are attractive building blocks for making higher order 2D arrays and 3D crystal protein assemblies, and can be useful for receptor clustering in cellular engineering³¹. We first set out to design dihedral protein assemblies of D2 symmetry. A set of C2 homo-oligomers with DHR termini (described above) were fused with select de novo hetero-dimers (tj18_asym13, unpublished work) using WORMS (schematics shown in FIGS. 4a-b). The D2 rings harbored total 8 protein chains with 2 chains (two-component) as the asymmetric unit. To generate these rings, we used a database of building blocks containing 7 homo-dimers and 1 heterodimer using the following configuration:

[(‘C2_C’,orient(None,‘C’)),(‘Het:CN’,orient(‘N’,‘C’)),
(‘C2_N’,orient(‘N’,None))
]
D2(c2=0, c2b=−1)

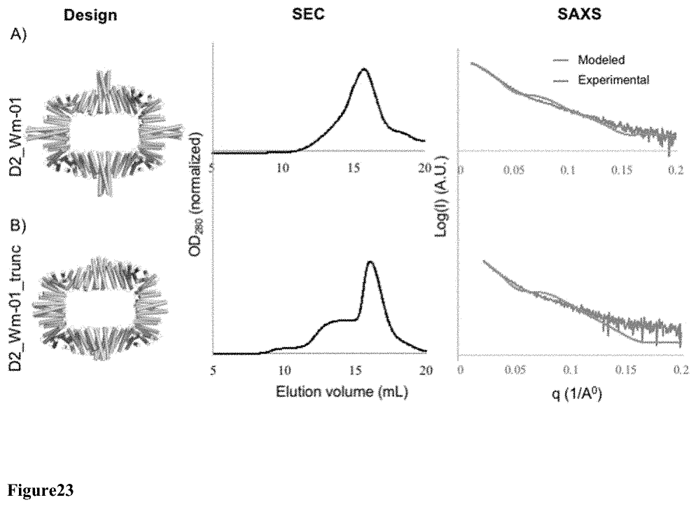

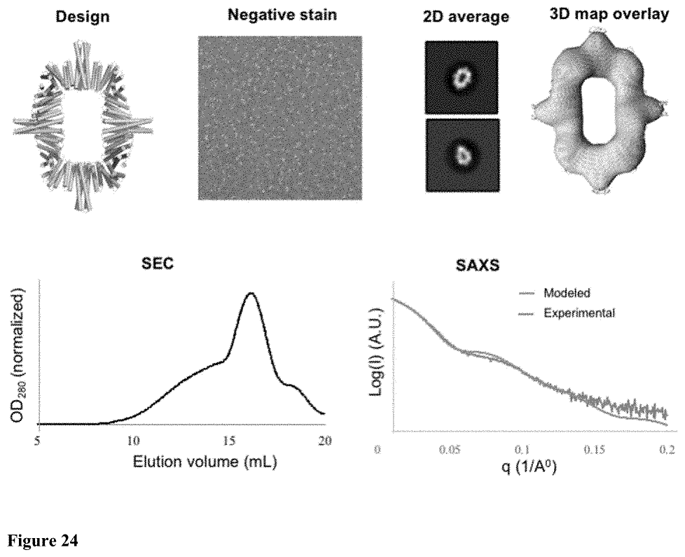

Of 208 outputs, we selected 6 designs to test, out of which three expressed as soluble two-component protein assemblies as indicated by Ni-NTA pulldown and subsequent SDS-PAGE experiments. Of these, two designs (designated as D2_Wm-01 and D2_Wm-02) eluted as expected by SEC and had SAXS profiles that matched with the designed models (FIG. 23-24).

To characterize the structures of D2 Wm-01 and D2 Wm-02 in more detail, we performed negative-stain EM and subsequent 2D averaging and 3D refinement. 2D averaging shows the resemblance of the designed model with the experiment-determined structures, whereas 3D refinement indicated accurate design of D2_Wm-01 and D2_Wm-02 at ˜16 Å resolution (FIG. 4c, FIG. 24).

The homo dimeric building blocks used in D2 Wm-01 and D2_Wm-02 have large interface areas (˜35 residues long; 5 heptads). We sought to reduce the interface area by truncating the helices to facilitate expression of the components and reduce off target interactions. Deletion of one heptad from either of the homodimers of D2_Wm-01 (designated D2_Wm-01_trunc) resulted in a single and much narrower SEC peak of the expected molecular weight (FIG. 23). Negative-stain EM followed by 2D averaging and 3D refinement indicated monodispersed particles with accurate structure as of the designed model (FIG. 4c).

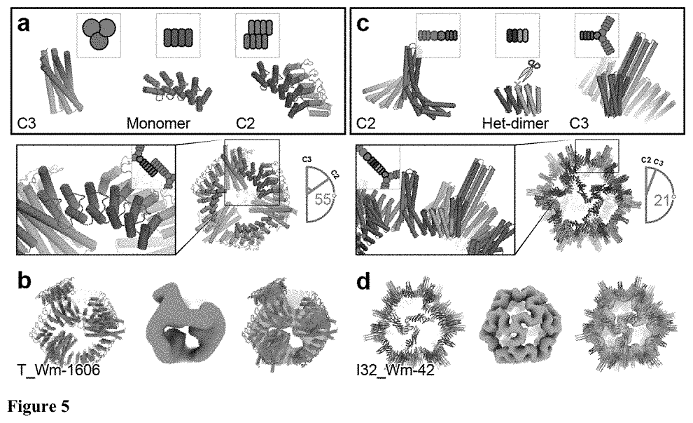

Generation of one-component tetrahedral protein cages: Idealized ankyrin homo-dimers²⁵ based on ANK1 and ANK3 and selected HBs²⁰ were combined to design one-component tetrahedral cages capable of hosting engineered DARPIN binding sites. For each combination, a monomeric ankyrin that perfectly matches the homo-dimer backbone was added as a spacer in between the homo-oligomers, thus extending the ankyrin homo-dimer by several repeats (FIG. 5a). To set up this architecture, the following configuration can be used:

[(‘C2_N’,orient(None,‘N’)),(‘Monomer’,orient(‘C’,‘N’)),(‘C3_C’,orient(‘C’,None))]

Tetrahedral(c2=0, c3=−1)

Due to the relatively small space of possibilities because of the limited building block set, only 27 valid fusion combinations were identified, of which 20 involved ankyrin homo-dimer extension at its N-terminus and the remaining 7 at its C-terminus. Eight (8) were selected by manual inspection for further sequence design at fusion regions and experimental characterization.

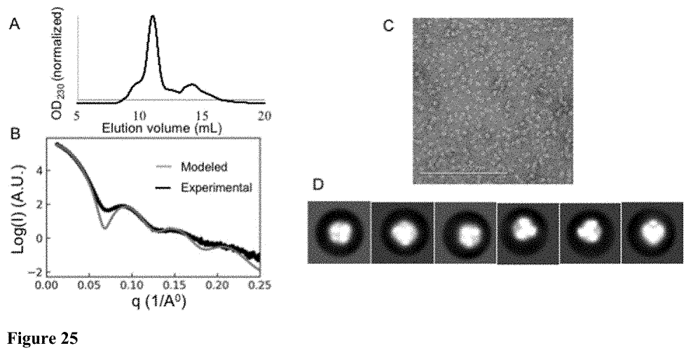

All 8 constructs were expressed and two were found to be soluble with mono-disperse elution profile peaks by SEC. The two promising structures were very similar, containing different helical bundles whose backbone geometry was identical, but with different internal hydrogen-bond networks. As the two were so similar, only one (T_Wm-1606) was selected for negative-stain EM and discrete particles were observed whose 2D class averages and 3D reconstruction to 20 Å matched the computational model (FIG. 5b). There was also good agreement between experimental SAXS profiles and profiles computed from the design model (FIG. 25).

Generation of two-component icosahedral protein cages: Point group symmetry nanocages have been successfully designed using docking followed by interface design⁵⁻⁷. To build such structure using our building blocks with the smaller and weaker interfaces that give rise to cooperative assembly³²⁻³⁴, we systematically split each DHR at the loop in the center of four repeats, resulting in a hetero-dimeric structure with two repeats on each side. The resulting interfaces are considerably smaller than in for example our de novo designed helical bundles. The WORMS protocol was then applied using the C5, C3, and C2 HelixFuse libraries described above at their corresponding tetrahedral, octahedral, and icosahedral symmetry axes. The split DHRs were then sampled to be connected in the center to each of the two symmetrical oligomers (FIG. 5c), using the configuration described above. Following fusion, sequence design was performed at each of the two new junctions.

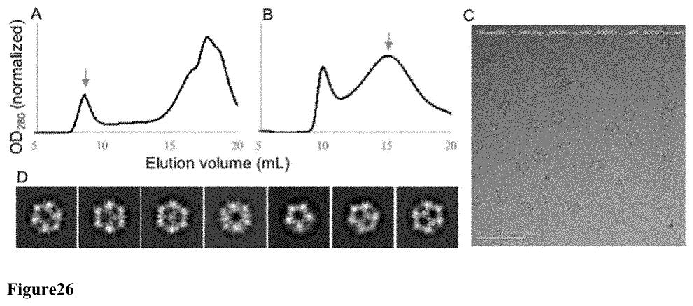

57 total designs were selected for experimental characterization; 25 co-eluted by Ni-NTA chromatography, and of these 7 designs had large peaks in the void volume in SEC chromatography as expected for particles of this size. When the peaks were collected and re-analyzed with a Sephacryl™ 500 column, one design, 132_Wm-42 (icosahedral architecture) was resolved into a void and a resolved peak (FIG. 26). Cryo-EM analysis of the resolved peak reveals well formed particles that when reconstructed to 9 Å resolution, accurately match the design model, including the distinct “S” shaped turn between the C3 and C2 axes (FIG. 5d). This structure is considerably more open than previous icosahedral cages built by designing non-covalent interfaces between homo-oligomers. For another design, T32_Wm-24, while cage was not formed, we were able to crystallize the polar-capped trimer component (C3_HF_Wm-0024A) and solve the structure by x-ray diffraction to 2.69 Å (FIG. 2B). The structure clearly shows that both of the newly designed junctions (from HelixFuse and WORMS) are as designed, matching the design model.

The 120 subunit I32_Wm-42 icosahedral nanocage has a molecular weight of 3.4 MDa and a diameter of 42.7 nm and illustrates the power of our combined hierarchical approach. I32_Wm-42 is constructed from five building blocks (two helical bundles and three repeat proteins) combined via four unique rigid junctions; the EM structure demonstrates that all were modeled with reasonable accuracy. The combination of the HelixDock and HelixFuse helix fusion methods created a large set of over 1500 oligomeric building blocks from which WORMS was able to identify combinations and fusion points that generated the icosahedral architecture; this example is notable because none of the oligomeric building blocks had been previously characterized experimentally. With fewer unknowns, either using less segments or a larger fraction of previously validated building blocks, we expect considerable improvement of the overall success rate.

### DISCUSSION

Our general rigid helix-fusion based pipeline provides a robust and accurate procedure for generating large protein assemblies by fusing symmetric building blocks and avoiding interface design, and should streamline assembly design for applications in vaccine development, drug delivery and biomaterials more generally. The set of structures generated here goes considerably beyond our previous work with rigid helical fusions, and the “WORMS” software introduced here is quite general and readily configurable to different nanomaterial design challenges. WORMS can be easily extended to other symmetric assemblies including 2D arrays and 3D crystals, and should be broadly useful for generating a wide range of protein assemblies.

DNA nanotechnology has had advantages in modularity and simplicity over protein design because the basic interactions (Watson-Crick base pairing) and local structures (the double helix) are always the same. Proteins in nature exhibit vast diversity compared to duplex DNA, and correspondingly, re-engineering naturally occurring proteins and designing new ones has been a more complex task than designing new DNA structures. The large libraries of “clickable” building blocks—helical bundle—repeat protein fusions—and the generalized WORMS software for assembling these into a wide range of user specifiable architectures that we present in this paper are a step towards achieving the modularity and simplicity of DNA nanotechnology with protein building blocks. Although this modularity comes at some cost in that the building blocks are less diverse than proteins in general, they can be readily functionalized by fusion to protein domains with a wide range of functions. We show that it is possible to genetically fuse DHR “adapters” to natural proteins; these proteins can then be used in larger assemblies through WORMS with less likelihood of disrupting the original protein fold. Proteins of biological and medical relevance (binders like protein A, enzymes, etc.) can be used as components and combined with de novo designed HBs and DHRs to form nanocages and other architectures.

### Computational Methods Summary:

Rosetta™ Remodel Forward Folding: To test the extent to which the designed sequences encode the designed structure around the junction site, we used large scale de novo folding calculations. Due to computational limitations with standard full chain forward folding^{36,37}, we developed a similar but alternate approach for larger symmetric structures. Using Rosetta™ Remodel²⁷ in symmetry mode (reversing the anchor residue for cases where the helical bundle was at the C-terminus), we locked all residues outside the junction region as rigid bodies, only allowing 40 residues starting from the end of the HB in the primary sequence direction of the DHR to be re-sampled. The blueprint file was set up to be agnostic of secondary structure in this segment of protein and we deleted all DHR residues past the first two helices after the rigid body region to reduce CPU cost. Each structure was set to at least 2000 trajectories to create a forward folding funnel.

WORMS: The WORMS software overall requires two inputs, a database of building block entries (format described in Supplementary Information in detail) and a configuration file (or command line options) as described in the main text to govern the overall architecture. While some segments can be of single building blocks of interest, to generate a wide variety of outputs, tens to thousands of entries per segment should be used. The number of designs generated also depends on the number of fusion points allowed, as the size of the space being sampled increases multiplicatively with the number of segments being fused. There are many options available to the user to control the fusions which are output as solutions; we have tuned the default options to be relatively general-use (see Supplementary Information for description of options). A key parameter is the tolerance, the allowed deviation of the final segment in the final structure away from its target position given the architecture. For different geometries the optimal values vary; for example the same tolerance values involve more drastic error in icosahedral symmetry than cyclic symmetry. The WORMS code is specifically designed to generate fusions that have a protein core around the fusion joint; unless specified using the ncontact_cut, ncontact_no_helix_cut, and nhelix_contacted_cut option set, the code will not produce single extended helix fusions.

### Brief Experimental Methods:

Gene preparation: All amino acid sequences derived from Rosetta™ were reverse translated to DNA sequences and placed in the pET29b+ vector. For two-component designs, all designs were initially constructed for bi-cistronic expression by appending an additional ribosome binding site (RBS) in front of the second sequence with only one of the components containing a 6xHis tag. Genes were synthesized by commercial companies: Integrated DNA Technologies (IDT), GenScript, Twist Bioscience, or Gen9.

Protein expression and purification: All genes were cloned into E. coli cells (BL21 Lemo21 (DE3)) for expression, using auto-induction³⁸ at 180 or 37° C. for 16-24 hours in 500 mL scale. Post-induction, cultures were centrifuged at 8,000×G for 15 minutes. Cell pellets were then resuspended in 25-30 mL lysis buffer (TBS, 25 mM Tris, 300 mM NaCl, pH8.0, 30 mM imidazole, 0.25 mg/mL DNase I) and sonicated for 2 minutes total on time at 100% power (10 sec on/off) (QSonica). Lysate was then centrifuged at 14,000×G for 30 minutes. Clarified lysates were filtered with a 0.7 um syringe filter and put over 1-4 mL of Ni-NTA resin (QIAgen), washed with wash buffer (TBS, 25 mM Tris, 300 mM NaCl, pH8.0, 60 mM imidazole), then eluted with elution buffer (TBS, 25 mM Tris, 300 mM NaCl, pH8.0, 300 mM imidazole). Eluate was then concentrated with a 10,000 m/w cutoff spin concentrator (Millipore) to approximately 0.5 mL based on yield for SEC.

D2 proteins went through an extra round of bulk purification. Concentrated protein was heated at 90° C. for 30 minutes to further separate bacterial contaminants. Samples were then allowed to cool down to room temperature and any denatured contaminants were removed by centrifuging at 20,000×G.

Size exclusion chromatography (SEC): All small oligomers were passed through a Superdex™ 200 Increase 10/300 GL column (Cytiva) while larger assemblies were passed through a Superose™ 6 Increase 10/300 GL column (Cytiva) on a AKTA PURE™ FPLC system. The mobile phase was TBS (TBS, 25 mM Tris, 300 mM NaCl). Additionally, for the icosahedral assembly, an additional custom packed 10/300 Sephacryl™ 500 column (Cytiva) was used to separate out the void. Samples were run at a speed of 0.75 mL/min and eluted with 0.5 mL fractions.

Protein Characterization: See supplementary information for detailed methods regarding SAXS sample preparation, electron microscopy, and x-ray crystallography.

### REFERENCES

1. Baker, D. What has de novo protein design taught us about protein folding and biophysics?Protein Sci. 28, 678-683 (2019).
2. Huang, P.-S., Boyken, S. E. & Baker, D. The coming of age of de novo protein design. Nature 537, 320-327 (2016).
3. Fallas, J. A. et al. Computational design of self-assembling cyclic protein homo-oligomers. Nat. Chem. 9, 353-360 (2017).
4. Sahasrabuddhe, A. et al. Confirmation of intersubunit connectivity and topology of designed protein complexes by native MS. Proc. Natl. Acad. Sci. 115, 1268-1273 (2018).
5. King, N. P. et al. Accurate design of co-assembling multi-component protein nanomaterials. Nature 510, 103-108 (2014).
6. Bale, J. B. et al. Accurate design of megadalton-scale two-component icosahedral protein complexes. Science 353, 389-394 (2016).
7. Hsia, Y. et al. Design of a hyperstable 60-subunit protein icosahedron. Nature 535, 136-139 (2016).
8. Shen, H. et al. De novo design of self-assembling helical protein filaments. Science 362, 705-709 (2018).
9. Gonen, S., DiMaio, F., Gonen, T. & Baker, D. Design of ordered two-dimensional arrays mediated by noncovalent protein-protein interfaces. Science 348, 1365-1368 (2015).
10. Ueda, G. et al. Tailored Design of Protein Nanoparticle Scaffolds for Multivalent Presentation of Viral Glycoprotein Antigens. http://biorxiv.org/lookup/doi/10.1101/2020.01.29.923862 (2020) doi:10.1101/2020.01.29.923862.
11. Marcandalli, J. et al. Induction of Potent Neutralizing Antibody Responses by a Designed Protein Nanoparticle Vaccine for Respiratory Syncytial Virus. Cell 176, 1420-1431.e17 (2019).
12. Butterfield, G. L. et al. Evolution of a designed protein assembly encapsulating its own RNA genome. Nature 552, 415-420 (2017).
13. King, N. P. et al. Computational design of self-assembling protein nanomaterials with atomic level accuracy. Science 336, 1171-1174 (2012).
14. Leaver-Fay, A. et al. Rosetta3. in Methods in Enzymology vol. 487 545-574 (Elsevier, 2011).
15. McConnell, S. A. et al. Designed Protein Cages as Scaffolds for Building Multienzyme Materials. ACS Synth. Biol. 9, 381-391 (2020).
16. Youn, S.-J. et al. Construction of novel repeat proteins with rigid and predictable structures using a shared helix method. Sci. Rep. 7, 2595 (2017).
17. Brunette, T. et al. Modular repeat protein sculpting using rigid helical junctions. Proc. Natl. Acad. Sci. 117, 8870-8875 (2020).
18. Vulovic, I. et al. Generation of ordered protein assemblies using rigid three-body fusion. http://biorxiv.org/lookup/doi/10.1101/2020.07.18.210294 (2020) doi:10.1101/2020.07.18.210294.
19. Huang, P.-S. et al. High thermodynamic stability of parametrically designed helical bundles. Science 346, 481-485 (2014).
20. Boyken, S. E. et al. De novo design of protein homo-oligomers with modular hydrogen-bond network-mediated specificity. Science 352, 680-687 (2016).
21. Chen, Z. et al. Programmable design of orthogonal protein heterodimers. Nature 565, 106-111 (2019).
22. Thomson, A. R. et al. Computational design of water-soluble α-helical barrels. Science 346, 485-488 (2014).
23. Brunette, T. et al. Exploring the repeat protein universe through computational protein design. Nature 528, 580-584 (2015).
24. Boyken, S. E. et al. De novo design of tunable, pH-driven conformational changes. Science 364, 658-664 (2019).
25. Fallas, J. A. et al. Computational design of self-assembling cyclic protein homo-oligomers. Nat. Chem. 9, 353-360 (2017).
26. Lawrence, M. C. & Colman, P. M. Shape complementarity at protein/protein interfaces. J. Mol. Biol. 234, 946-950 (1993).
27. Huang, P.-S. et al. RosettaRemodel: A Generalized Framework for Flexible Backbone Protein Design. PLoS ONE 6, e24109 (2011).
28. Coventry, B. & Baker, D. Protein sequence optimization with a pairwise decomposable penalty for buried unsatisfied hydrogen bonds. http://biorxiv.org/lookup/doi/10.1101/2020.06.17.156646 (2020) doi:10.1101/2020.06.17.156646.
29. Fullerton, S. W. B. et al. Mechanism of the Class I KDPG aldolase. Bioorg. Med. Chem. 14, 3002-3010 (2006).
30. Geiger-Schuller, K. et al. Extreme stability in de novo-designed repeat arrays is determined by unusually stable short-range interactions. Proc. Natl. Acad. Sci. 115, 7539-7544 (2018).
31. Correnti, C. E. et al. Engineering and functionalization of large circular tandem repeat protein nanoparticles. Nat. Struct. Mol. Biol. 27, 342-350 (2020).
32. Zlotnick, A. To Build a Virus Capsid. J. Mol. Biol. 241, 59-67 (1994).
33. Zlotnick, A., Johnson, J. M., Wingfield, P. W., Stahl, S. J. & Endres, D. A Theoretical Model Successfully Identifies Features of Hepatitis B Virus Capsid Assembly^{†}. Biochemistry 38, 14644-14652 (1999).
34. Ceres, P. & Zlotnick, A. Weak Protein-Protein Interactions Are Sufficient To Drive Assembly of Hepatitis B Virus Capsids^{†}. Biochemistry 41, 11525-11531 (2002).
35. Padilla, J. E., Colovos, C. & Yeates, T. O. Nanohedra: Using symmetry to design self assembling protein cages, layers, crystals, and filaments. Proc. Natl. Acad. Sci. 98, 2217-2221 (2001).
36. Marcos, E. et al. Principles for designing proteins with cavities formed by curved p sheets. Science 355, 201-206 (2017).
37. Marcos, E. & Silva, D.-A. Essentials of de novo protein design: Methods and applications. Wiley Interdiscip. Rev. Comput. Mol. Sci. 8, e1374 (2018).
38. Studier, F. W. Protein production by auto-induction in high-density shaking cultures.

### Supporting Information

### Supplementary Methods

### HelixDock Details

HelixDock designs were first also attempted as “two-body hydrophobic” (2BH) designs, where the extra loop was not added to connect the helical bundle and repeat protein chain termini after a hydrophobic interface was designed between them. A couple dozen designs were tested in this fashion, but they all poor solubility or showed highly heterogeneous assemblies by SEC. This was hypothesized to be caused by the weak association of the repeat protein to the helical bundle; the final assembly was in constant equilibration and did not maintain the full stoichiometry. All further attempts with HelixDock were designed as “one-body hydrophobic” (1BH), where the helical bundle and repeat protein were closed into a single chain, as described in the main text. The HelixDock protocol is broken down into three distinct parts: docking, interface design, and loop closure.

### HelixDock: Docking

The docking was performed using a modified version of the sicdock app, as previously described to generate cyclic oligomers from monomers. A new type of symmetry was added on to allow the docking of monomers in a symmetric manner (in this case, a repeat protein monomer) in all 6 degrees of freedom (dof) to a symmetry matching oligomer in the center that is not perturbed (in this case, a helical bundle). This final resulting architecture can be described as two matching cyclic symmetries stacked on top of each other along the z-axis; this definition will be used again as a symdef file during Rosetta™ Design. The docks were filtered based on their motif score, which is an estimate of the interface size and the likelihood of the dock to generate a decent interface post-design. An additional filter was used to make sure the termini between the helical bundle and repeat protein were compatible; the N- and C-terminus needed to be within 9 angstroms of one another.

### HelixDock: Sequence Design and Loop Closure

To model the dock output correctly in Rosetta™, we generated new symdef files which allowed the architecture as described above. Using SymDofMover™, we were able to regenerate the docked conformation and select the relevant residues for interface design and subsequent filtering. To close the termini between the helical bundle and repeat protein, the ConnectChainsMover™ was used. After loop closure, residues at and around the new loop were redesigned and scored to ensure compatibility. Example *.sym and *.xml files that accomplishes these steps are available in the supplemental materials.

### WORMS Relevant Command Line Options:

The full list of available command line options can be initiated with --help.

TABLE 2
I/O options

| Option | Default | Description |
|---|---|---|
| --geometry | | Specifies geometry (see |
| | | architecture below). |
| --bbconn | | Specifies connectivity |
| | | (see architecture below). |
| --config_{—}file | | Specifies the config file |
| | | to be used (see |
| | | architecture below). |
| --nbblocks | 64 | The maximum number of |
| | | building blocks that can |
| | | be used in each segment. |
| --dbfiles | | Space delimited list of |
| | | database files to be read |
| | | in (see databases below). |
| --shuffle_{—}bblocks | 1 | Uses a random set of |
| | | building blocks instead of |
| | | sequential from the top; |
| | | relevant only if nbblocks < |
| | | total actual bblocks |
| | | available in that segment. |
| --max_{—}output | 1000 | Maximum number of pdbs to |
| | | output. |

TABLE 3
Splice Level Filtering options

| Option | Default | Description |
|---|---|---|
| --no_{—}duplicate_{—}bases | 1 | Prevents duplicated ‘bases’ in the final structure |
| | | (see databases below). |
| --min_{—}seg_{—}len | 15 | Minimum length required for each segment in the |
| | | fusion. |
| --splice_{—}rms_{—}range | 5 | During splicing, this specifies the number of |
| | | residues to check for rms at the fusion junction. |
| | | (+/−) this many residues. |
| --splice_{—}max_{—}rms | 0.7 | Maximum rms allowed at the fusion junction. |
| --splice_{—}ncontact_{—}cut | 38 | Minimum number of contacts required across the |
| | | interface. |
| --splice_{—}ncontact_{—}no_{—}helix_{—}cut | 6 | Minimum number of contacts required across the |
| | | interface after removing the fusion helix. This |
| | | filters against fusions where there are no |
| | | additional interactions between the segments. |
| --splice_{—}nhelix_{—}contacted_{—}cut | 3 | Minimum number of helices in contact after |
| | | removing the fusion helix. |
| --splice_{—}max_{—}chain_{—}length | 450 | Maximum final chain length after fusion. |
| --tolerance | 1.0 | Angstrom deviation from final structure, |
| | | respective to its ideal axis. |

TABLE 4
PyRosetta ™ Level Filtering Options

| | | |
|---|---|---|
| --max_{—}score0 | 4.0 | Asymmetric Score0 filter after Rosetta ™ scoring. |
| --full_{—}score0sym | 1.0 | Symmetric Score0 filter after Rosetta ™ scoring. |
| --max_{—}com_{—}redundancy | 4.0 | Computes the center of mass for each segment |
| | | and filters designs out if the same building block |
| | | is used at the same segments and their center of |
| | | mass are in similar positions. |
| --postfilt_{—}splice_{—}rms_{—}length | 9 | PyRosetta ™ version of --splice_{—}rms_{—}range |
| --postfilt_{—}splice_{—}max_{—}rms | 0.7 | PyRosetta ™ version of --splice_{—}max_{—}rms |
| --postfilt_{—}splice_{—}ncontact_{—}cut | 40 | PyRosetta ™ version of --splice_{—}ncontact_{—}cut |
| --postfilt_{—}splice_{—}ncontact_{—}no_{—}helix_{—}cut | 2 | PyRosetta ™ version of --splice_{—}ncontact_{—}no_{—}helix_{—}cut |
| --postfilt_{—}splice_{—}nhelix_{—}contacted_{—}cut | 3 | PyRosetta ™ version of --splice_{—}nhelix_{—}contacted_{—}cut |

### WORMS Architecture Definition:

The worms architecture can be definition in two different methods, either as an *.config file or as command line options. Described immediately below is the *.config file syntax.

[‘C3_N’,orient(None,‘N’)),(‘Het:CN’,orient(‘C’,‘N’)),
(‘C2_C’,orient(‘C’,None)
]

The first line of the *.config file defines the connections between all the segments, and which building blocks are allowed in each segment, as described in the main text. Marked in bold is a single ‘segment’ of the worm. The first field is the ‘name’, ‘class’ or ‘type’ of the building block(s) that are desired in that segment (see database syntax for more information). The next field is ‘orient(x,y)’, which defines which connections are to be used. The termini assigned here will limit the search in the building block database to those who have that termini available. On the first and last segments, the notation ‘None’ is used to signify that there are no additional connections on that side. The segments in the center need two assignments to which termini are to be connected—this can be ‘C’ or ‘N’, depending on what is available. In the case of a monomer, a single ‘C’ and a single ‘N’ is available. For a hetero-dimer, however, ‘N’,‘N’ or ‘C’,‘C’ assignments are possible. Keep in mind that a ‘N’ must connect to a ‘C’ in the next segment, and vice versa.

At present, the WORMS software supports the following architectures:

Cyclic(symmetry=1)
D2(c2=0, c2b=−1)
D3(c3=0, c2=−1)
D4(c4=0, c2=−1)
D5(c5=0, c2=−1)
D6(c6=0, c2=−1)
Icosahedral(c5=None, c3=None, c2=None)
Octahedral(c4=None, c3=None, c2=None)
Tetrahedral(c3=None, c3b=None, c2=None)

The variable values listed here are the default values; they can be changed to what the user requires. For ‘Cyclic’, the symmetry variable determines what the overall oligomeric state is. For example, ‘symmetry=3’ will generate a C3 architecture. For the remaining architectures, the variables are to assign the terminal segments to their respective symmetry axis. In the ‘D3’ case, the C3 component is assigned to the ‘0th’ segment, which is the first segment listed above. The C2 component is assigned to the ‘−1th’ segment, which is the last segment listed above.

To use the command line format, the following syntax is used, a D2 architecture is shown as an example:

```
--geometry D2(c2a=0, c2b=−1)
--bbconn
  _C C2_C
  NC Monomer
  N_ C2_N
```

The major syntax difference is that instead of ‘None’, a single underscore ‘_’ is used in place for the first and last segment connections.

### WORMS Database Syntax and Example Entries:

```
[
{“file”: “/path/to/pdb/file1.pdb”,
  “name”: “symmetric_cyclic_example_0001” ,
  “class”: [“C3_C”],
  “type”: “example_C3_C” ,
  “base”: “base_scaffold” ,
  “components”: [“component1”,“component2”],
  “validated”: false,
  “protocol”: “made_by_example_protocol”,
  “connections”: [
      {“chain”: 1, “direction”: “C”, “residues”:[“−129:”]},
      {“chain”: 1, “direction”: “N”, “residues”:[“:180”]}
  ]
},
{“file”: “/path/to/pdb/file2.pdb”,
  “name”: “asymmetric_het_example_0001” ,
  “class”: [“Het”],
  “type”: “example_het_C2_C-C” ,
  “base”: “base_scaffold” ,
  “components”: [“component1”, “component2”],
  “validated”: false,
  “protocol”: “made_by_example_protocol”,
  “connections”: [
      {“chain”: 1, “direction”: “C”, “residues”:[“−129:”]},
      {“chain”: 1, “direction”: “N”, “residues”:[“:150”]},
      {“chain”: 2, “direction”: “C”, “residues”:[“−139:”]},
      {“chain”: 2, “direction”: “N”, “residues”:[“:86”]},
    ]
}
]
```

While not all variables need to be populated (only file, name, class, and connections are required), the other variables allow the user to customize their search of building blocks during the WORMS run. The user can specify a specific name in the configuration which will result in that segment being populated by a single building block. Alternatively, by searching with class or type, the user can specify that segment to be any entry that contains the desired keyword. For hetero-oligomeric entries, the class keyword “Het” is used. During the configuration setup (see above), the user can specify what kind of hetero-oligomer is desired:

‘Het: CN’—all hetero-oligomers that have at least 1 C- and 1 N-term available.
‘Het:CNX’—only hetero-trimers, even if you do not require the 3rd terminus
‘Het:CNY’—only hetero-dimers.

The base field can be used in conjunction with the --no_duplicate_bases option to make sure that in a single completed architecture there will not be the same base used in non-symmetrical positions. The components, validated, and protocols fields are strictly for filtering purposes.

The connections field is where the user populates direction, which depicts which termini are available in each chain in a given entry. In the residues field, the user specifies which residues are allowed to be sampled as fusion positions. The numbering follows standard python syntax, for example, [:100] equates to the range: “first residue to residue 100”, and [−100:] equates to the range: “last 100 residues from the end to the end”.

### WORMS Sequence Design:

All outputs from WORMS were sequence-designed using Rosetta™ Scripts with rigid backbone. The residues that need to be designed can be found appended to the WORMS asymmetric unit *.pdb output. These were identified as residues which either “gained a new contact” or “lost an old contact” in the new fused WORMS context. Each chain from the WORMS output was designed separately for computational runtime purposes, under the assumption that the junction regions are not close to one another. Afterwards, all the designed chains were then combined and designed in the symmetrical context to remove residual clashing residues. Example *.xml files can be found as supplemental files.

### Small Angle X-Ray Scattering (SAXS):

Sample handling and SAXS experiments were performed according to previous methods¹. Briefly, proteins were SEC-purified in 25 mM Tris pH 8.0, 150 mM NaCl and 2% glycerol. Purified proteins collected from SEC-fractions were passed through MWCO filter columns (3 or 10 kDa cut off) to concentrate the protein samples, where the passed-through solutions were used as blanks for buffer subtraction. Scattering measurements were performed at the SIBYLS™ 12.3.1 beamline at the Advanced Light Source. The sample-to-detector distance was 1.5 m, and the X-ray wavelength (λ) was 1.27 Å, corresponding to a scattering vector q (q=4π sin θ/λ, where 20 is the scattering angle) range of 0.01 to 0.3 Å⁻¹. A series of exposures were taken of each well, in equal subsecond time slices: 0.3-s exposures for 10 s resulting in 32 frames per sample. Data were collected for two different concentrations for each sample: ‘low’ concentration samples ranged at 1-3 mg/ml and ‘high’ concentration samples at 2-6 mg/ml. Data was processed using the SAXS FrameSlice™ online server and analysed using the ScÅtter™ software package². Experimental scattering profiles to design models were compared using the FoXS™ online server³.

### X-Ray Crystallography:

X-ray crystallography Crystallization All crystallization trials were carried out at 20° C. in 96-well format using the sitting-drop method. Crystal trays were set up using Mosquito™ LCP by SPTLabtech. Drop volumes ranged from 200 to 400 nl and contained protein to crystallization solution in ratios of 1:1, 2:1 and 1:2. Diffraction quality crystals appeared in 0.2M Sodium chloride, 0.1M Sodium/Potassium phosphate pH 6.2 and 50% PEG200 (JCSG+ D3) for C3_HDock-1069; 0.2 M Lithium sulfate, 0.1M Na-acetate pH 4.5 and 2.5 M NaCl for C3_nat_HF-0005; 0.2 M MgCl₂, 0.1 TrisCl pH 8.5, 10% Glycerol and 25% (v/v) 1,2-Propanediol for C3_HF_Wm-0024A; and 0.1M MES pH 5.0, 20% MPD plus an additional 20% MPD as a cryoprotectant for C3_Cm-05. Crystals were subsequently harvested in a cryo-loop and flash frozen directly in liquid nitrogen for synchrotron data collection.

X-ray crystallography Data Collection Data collection from crystal of C3_nat_HF-0005 was performed with synchrotron radiation at the Advanced Photon Source (APS), 24ID-E. Crystals belonged to space group R 3:H with cell dimensions a=b=101.97 Å, and c=78.44 Å, α=β90° and γ=120°. Data collection from the crystal of C3_HF_Wm-0024A was performed with synchrotron radiation at the Advanced Light Source (ALS), 8.2.2. Crystals belonged to space group P43212 with cell dimensions a=b=166.77 Å, and c=223.51 Å, α=β=γ=90°. X-ray intensities and data reduction were evaluated and integrated using XDS⁴ and merged/scaled using Pointless/Aimless in the CCP4 program suite⁵.

Structure determination and refinement Starting phases were obtained by molecular replacement using Phaser⁶ using the designed model for the structures. Following molecular replacement, the models were improved using Phenix™ autobuild⁷; efforts were made to reduce model bias by setting rebuild-in-place to false, and using simulated annealing and prime-and-switch phasing. Structures were refined in Phenix™. Model building was performed using COOT⁸. The final model was evaluated using MolProbity⁹. Data collection and refinement statistics are recorded in Table S1.

### Electron Microscopy: Cyclic Structures (C4, C5 and C6)

Negative stain EM grid preparation, data collection, and data processing Proteins were diluted to 20 μg/ml with TBS, then immediately applied to freshly glow-discharged Formvar™/carbon 400 mesh copper grids (Ted Pella catalog #01754-F). After incubation for 45s, excess protein solution was removed by blotting from the side with filter paper, then grids were inverted onto two successive drops of sample buffer followed by three to five successive drops of 2% uranyl formate, with excess solution removed by blotting after each application. The final stain applied was incubated for 15s before blotting. Air-dried grids were imaged using a FEI Talos™ L120C TEM equipped with a 4K×4K Gatan OneView™ camera, at a nominal magnification of 73,000× and pixel size of 2.0 Å. Micrographs were imported to Relion™ 3.11 and/or cryoSPAR.C™ v2¹¹ and, after picking using automated protocols in each program, particles were subjected to 2D classification. Design model projections were generated using EMAN2™¹² and Relion™, and projections were aligned with experimental 21) class averages using Sparx¹³.

Cryo-EM grid preparation and data collection 3.5 μL of C4_nat_HF-7900 at a concentration of 1 mg/ml was applied to 400 mesh copper Quantifoil™ holey carbon grids 1.2/1.3 coated with graphene oxide (catalog #GOQ400R1213Cu, Electron Microscopy Sciences). C5_HF-3921 and C5_HF-0019 were diluted with TBS to final concentrations of 0.75 mg/ml and 0.45 mg/ml, respectively, immediately before applying to glow-discharged 400 mesh copper Quantifoil™ holey carbon grids 1.2/1.3 (3.5 μL of C5_HF-3921 and 3.0 μL of C5_HF-0019). All grids were plunge-frozen using a Vitrobot™ Mark IV. Grids were pre-screened on a Talos Arctica™ microscope operated at 200 kV with a Gatan K3™ camera (NYU) and C4_nat_HF-7900 movies were collected with this setup. C5_HF-3921 movies were acquired on a Titan Krios™ microscope (“Krios™ 3”) operated at 300 kV with Gatan K3™ camera and located at the New York Structural Biology Center. To address preferred orientation of particles, C5_HF-3921 movies were acquired at both 0° and 35° tilt angles, and for tilted movies a 4s pre-exposure wait time was added. Data acquisition was controlled via Leginon¹⁴ and pre-processing was performed with Appion¹⁵. Data collection parameters are shown in Supplementary Table S2.

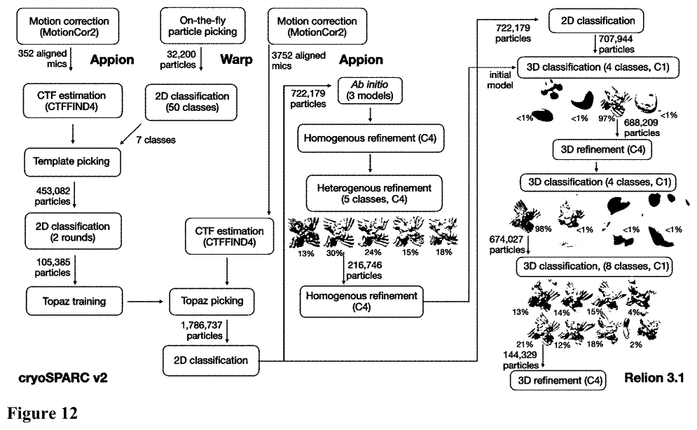

Cryo-EM data processing Detailed processing workflows are shown in FIGS. 12 and 14. Movies were motion-corrected and dose-weighted using MotionCor2™¹⁶ within Leginon/Appion, then imported to cryoSPARC™ v2 for CTF estimation, particle picking, 2D classification, and ab inilio 3D reconstruction. For C4_nat_HF-7900, particles picked “on-the-fly” with Warp™¹⁷ were imported to cryoSPAR_C™ for 2D classification to generate particles to use as templates for template-based picking. Multiple rounds of 2D classification and manual curation were used to generate a set of particles to use as a training set for Topaz™¹⁸. Topaz-picked particles were then used for further 2D/3D classification and 3D refinement in cryoSPARC™. For C5 HF-3921, images collected at both 0° and 35° were processed together following patch CTF estimation for 2D classification, ab initio 31) reconstruction, and initial 3D refinement. The best C5_HF-3921 map resulted from 3D refinement of data collected at a 350 tilt angle only. For C4_nat_HF-7900, after initial processing in cryoSPARC™, particles picked by Topaz™ were imported to Relion™ 3.1 for further 2D/3D classification and 3D refinement. 3D refinements were performed both with and without symmetry imposed. For C4_nat_HF-7900, imposing C4 symmetry yielded the highest quality map, whereas for C5_HF-3921 a C1 map had higher overall quality despite lower nominal resolution (due to artifacts introduced by imposing C5 symmetry). Overall map resolutions were estimated using the gold-standard Fourier Shell Correlation criterion (FSC=0.143) within Relion™ (C4_nat_HF-7900) or cryoSPARC™ (C5_HF-3921) and 3D FSC were calculated using the “Remote 3DFSC Processing Server”¹⁹ Soft masks were provided for estimation of local resolution of C4_nat_HF-7900 and C5_HF-3921 maps using implementations within Relion™ and cryoSPARC™, respectively.

C4_nat_HF-7900 model building and refinement The ab initio coordinates of the C4_nat_HF-7900 design were used as the starting model. Four C4_nat_HF-7900 protomers were first individually docked into the cryo-EM map as rigid bodies using Chimera²⁰, then refined using iterative rounds of refinement with real_space_refine in PHENIX™⁷ followed by manual model adjustment in COOT™^{21,22}. Each of the four chains in the tetramer was divided into 2 rigid bodies (residues 1-65 and 66-295; corresponding to the HB and DHR, respectively). Rigid body and ADP refinement were performed, with secondary structure, non-crystallographic symmetry, Ramachandran, and rotamer restraints enabled. The model was then analyzed using COOT™ and residues 261-295 were removed due to weak density in this region of the cryo-EM map. Since the C-terminus of C4_nat_HF-7900 is >95% identical to a previously characterized DHR (PDB ID: 5cwp²³), secondary structure restraints for residues 71-260 were based on the 5cwp structural model. After multiple iterations of real_space_refine and manual model adjustment, all helices except for the two C-terminal helices in the model (residues 212-260) were well-placed within the cryo-EM map density. Inspection of the model and map showed ambiguity in the position of residues 210-213 due to low local resolution and discontinuous density in this region. This loop and the following two C-terminal helices were shifted relative to their position in the 5cwp structure, possibly as a result of incorrect Thr210-Pro213 loop placement. To determine whether this shift reflected a true difference between the C4_nat_HF-7900 DHR and 5cwp structures, we used the 5cwp structural model to drive placement of these helices as follows: 5cwp was aligned to residues 101-260 of the working C4_nat_HF-7900 model (excluding the N-terminal DHR helix in case of distortions introduced from fusion to the HB) and a hybrid model was created by joining residues 1-208 of C4_nat_HF-7900 to residues 140-191 of 5cwp using Chimera. The single amino acid difference between C4_nat_HF-7900 and 5cwp in the grafted C-terminus was mutated to restore the C4_nat_HF-7900 design sequence, and this model was subjected to additional rounds of PHENIX real_space_refine and manual refinement in COOT. After refinement, the backbone of the Thr210-Pro213 loop and C-terminal helices remained in position, leading to close alignment of C4_nat_HF-7900 residues 101-260 with 5cwp and a better fit of the two C-terminal helices to the cryo-EM map density.

Electron Microscopy: Higher Order Structures (Crowns, Dihedrals, and the Point Group Cages):

Negative-stain electron microscopy (NS-EM) Negative-stained sample grids for transmission electron microscopy were prepared using either Nano-W™ or Uranyl Formate (Nanoprobes) at a sample concentration of 0.01-0.005 mg/mL using manufacturer's standard operating procedure. Stained grids were screened using FEI Morgagni™ transmission electron microscope operating at 100 kV. For 2D averaging, images were collected in a Tecnai T12 electron microscope using Leginon™ image collection software. The parameters of the contrast transfer function (CTF) were estimated using CTFFIND4. All particles were picked in a reference-free manner using DoG Picker™. Reference-free 2D classification was used to select homogeneous subsets of particles using CryoSPARC™. The selected particles were subsequently subjected to ab initio 3D reconstructions and Homogenous 3D refinement using CryoSPARC™.

Cryo-electron microscopy 3 μL of 1 mg ml-1 of C5_Crn_HF_12_26 was loaded onto a freshly glow-discharged (30 s at 20 mA) 1.2/1.3 UltraFoil™ grid (300 mesh) prior to plunge freezing using a Vitrobot™ Mark IV (ThermoFisher Scientific) using a blot force of 0 and 6 second blot time at 100% humidity and 25° C. Data was acquired using an FEI Glacios™ transmission electron microscope operated at 200 kV and equipped with a Gatan K2™ Summit direct detector. Automated data collection was carried out using Leginon at a nominal magnification of 36,000× with a pixel size of 1.16 Å. The dose rate was adjusted to 8 counts/pixel/s, and each movie was acquired in counting mode fractionated in 50 frames of 200 ms. 1,709 micrographs were collected with a defocus range between −1.0 and −3.5 m. Movie frame alignment, estimation of the microscope contrast-transfer function parameters, particle picking, and extraction were carried out using Warp™. Reference-free 2D classification was used to select homogeneous subsets of particles using CryoSPARC™. The selected particles were subsequently subjected to ab initio 3D reconstructions and 3D refinements using CryoSPARC™ 3 μL of 1 mg ml-1 of I32_Wm-42 was loaded onto a freshly glow-discharged (30 s at 20 mA) 2.2 um c-flat grid prior to plunge freezing using a Vitrobot™ Mark IV (ThermoFisher Scientific) using a blot force of 0 and 6 second blot time at 100% humidity and 25° C. Data were acquired using an FEI Glacios™ transmission electron microscope operated at 200 kV and equipped with a Gatan K2™ Summit direct detector. Automated data collection was carried out using Leginon™ at a nominal magnification of 36,000× with a pixel size of 1.16 Å. 618 micrographs were collected with a defocus range between −1.2 μm and −3.5 μm. Movie frame alignment and estimation of the microscope contrast-transfer function parameters were carried out using Warp™. 500 particles were picked initially and 2D classifications were performed in cisTEM™. Eleven representative 2D class averaged images were selected as references for automatic particle picking. 2D classifications were performed in RELION™ 3.0. The selected particles were subsequently subjected to ab initio 3D reconstructions using CryoSPARC™ 3D classification and 3D refinements were performed using RELION™ 3.0.

### Supplementary Tables

TABLE S1
Crystallographic Data Collection and Refinement Statistics

| | C3_nat_HF-0005 | | C3_HF_Wm-0024A | | C3_HD-1069 | | C3_Crn-05 | |
|---|---|---|---|---|---|---|---|---|
| | (PDB: 6XH5) | | (PDB: 6XI6) | | (PDB: 6XT4) | | (PDB: 6XNS) | |
| Data collection | | | | | | | | |
| Space group | P4₃2₁2 | | R3: H | | R3: H | | P22₁2₁ | |
| Cell dimensions | | | | | | | | |
| a, b, c (Å) | 166.77, 166.77, 223.51 | | 101.97, 101.97, 78.44 | | 107.31, 107.31, 56.06 | | 112.13, 145.25, 161.89 | |
| a, β, γ (°) | 90, 90, 90 | | 90, 90, 120 | | 90, 90, 120 | | 90, 90, 90 | |
| Resolution (Å) | 78.12-3.32 | (3.43-3.32)^{a} | 38.48-2.69 | (2.78-2.69) | 35.78-2.4 | (2.486-2.4) | 46.39-3.19 | (3.30-3.19) |
| No. of unique | 47181 | (4621) | 8434 | (844) | 9405 | (928) | 44729 | (4436) |
| reflections | | | | | | | | |
| R_{merge} | 0.238 | (1.824) | 0.071 | (0.577) | 0.139 | (0.467) | 0.098 | (2.98) |
| Rₚᵢₘ | 0.046 | (0.348) | 0.038 | (0.324) | 0.06529 | (0.216) | 0.035 | (1.048) |
| I/σ(I) | 17.98 | (2.09) | 11.4 | (2.3) | 6.13 | (2.26) | 13.18 | (0.93) |
| CC_{1/2} | 0.986 | (0.723) | 0.997 | (0.889) | 0.993 | (0.922) | 0.999 | (0.247) |
| Completeness (%) | 99.88 | (99.98) | 99.59 | (99.41) | 99.27 | (99.15) | 99.79 | (99.57) |
| Redundancy | 27.2 | (28.4) | 4.8 | (4.8) | 5.6 | (5.6) | 8.9 | (9.0) |
| Refinement | | | | | | | | |
| Resolution (Å) | 78.12-3.32 | | 38.48-2.69 | | 35.78-2.4 | | 46.39-3.19 | |
| No. of reflections | 47141 | | 8412 | | 9338 | | 44729 | |
| R_{work}/R_{free} (%) | 22.17/26.48 | (30.12/37.64) | 22.10/27.72 | (33.81/36.32) | 22.75/27.30 | (30.82/34.85) | 27.08/29.56 | (41.15/40.11) |
| No. atoms | 15883 | | 2056 | | 1618 | | 12559 | |
| Protein | 15834 | | 2043 | | 1614 | | 12559 | |
| Water | 49 | | 13 | | 4 | | 0 | |
| Ramachandran | 95.47/4.29 | | 98.50/1.50 | | 99.56/0.44 | | 98.77/1.23 | |
| Favored/allowed | 00.24 | | 00.00 | | 00.00 | | 00.00 | |
| Outlier (%) | | | | | | | | |
| R.m.s. deviations | | | | | | | | |
| Bond lengths (Å) | 0.002 | | 0.001 | | 0.004 | | 0.002 | |
| Bond angles (°) | 0.470 | | 0.330 | | 0.56 | | 0.47 | |
| B_{factors} (Å²) | | | | | | | | |

| Protein | 117.47 | 74.96 | 54.58 | 130.31 |
|---|---|---|---|---|
| Water | 89.90 | 69.13 | 53.08 | — |

TABLE S2
CryoEM data collection parameters for C4_nat_HF-7900 and C5_HF-3921

| | C4_nat_HF-7900 | | C5_HF-3921 | |
|---|---|---|---|---|
| | (6XSS, EMD-22305) | | (EMD-22306) | |
| Microscope | Talos Arctica ™ | | Titan Krios ™ | |
| Electron energy | 200 | kV | 300 | kV |
| Pixel size | 0.859 | Å | 1.083 | Å |
| Total electron dose | 57.05 | e⁻/Å² | 68.61 | e⁻/Å² |
| Number of frames in each movie | 40 | | 50 | |
| Exposure time | 2800 | ms | 2500 | ms |
| Defocus range | −0.2-−4.2 | μm | −1.9-−5.0 | μm |
| Tilt angle(s) | 0° | | 0, 35° | |
| Number of images acquired | 3,752 | | 6,744 | |
| Number of particles used in final map | 144,329 | | 30,659 | |
| Final map resolution (FSC = 0.143) | 3.70 | | 8.06 | |
| B-factor for map sharpening | −180 | Å² | −500 | Å² |

| | | |
|---|---|---|
| Sphericity of 3DFSC | 0.895 | 0.786 |
| EMDB entry number (map) | EMD-22305 | EMD-22306 |
| EMPIAR entry number (data) | XXX | XXX |

TABLE S3
Model statistics for C4_nat_HF-7900 cryoEM structure

| | |
|---|---|
| Map CC (mask) | 0.78 |
| Map CC (volume) | 0.77 |
| Map CC (peaks) | 0.63 |
| rmsd (bonds) | 0.003 Å |
| rmsd (angles) | 0.605° |
| Ramachandran plot values | |
| outliers | 0.00% |
| allowed | 2.52% |
| favored | 97.48% |
| Rotamer outliers | 0.00% |
| C-beta deviations | 0.00% |
| Overall score (Molprobity¹⁶) | 2.04 |
| PDB ID | 6XSS |

### Design Construct Renaming

### HelixDock

| Published name | Original Name |
|---|---|
| C2_HD-1091 | YH_1BH-91 |
| C2_HD-1092 | YH_1BH-92 |
| C2_HD-1093 | YH_1BH-93 |
| C2_HD-1096 | YH_1BH-96 |
| C3_HD-1005 | YH_1BH-05 |
| C3_HD-2019 | UN_1BH-19 |
| C3_HD-1046 | YH_1BH-46 |
| C3_HD-1053 | YH_1BH-53 |
| C3_HD-1058 | YH_1BH-58 |

| Published name | Original Name |
|---|---|
| C3_HD-1064 | YH_1BH-64 |
| C3_HD-1066 | YH_1BH-66 |
| C3_HD-1068 | YH_1BH-68 |
| C3_HD-1069 | YH_1BH-69 |
| C6_HD-1010 | YH_1BH-10 |
| C6_HD-1011 | YH_1BH-11 |
| C6_HD-1013 | YH_1BH-13 |
| C6_HD-3014 | C6-14 |
| C6_HD-3019 | C6-19 |

| Published name | Original Name |
|---|---|
| C5_HF-2101 | C5-21-01 |
| C5_HF-3921 | C5-39-21 |
| C5_HF-0007 | C5_HFuse-0007 |
| C5_HF-0016 | C5_HFuse-0016 |
| C5_HF-0019 | C5_HFuse-0019 |
| C5_HF-0032 | C5_HFuse-0032 |
| C6_HF-0069 | C6-69 |
| C6_HF-0075 | C6-75 |
| C6_HF-0080 | C6-80 |
| C3_nat_HF-0005 | 1wa3_HFuse_BA-05 |
| C4_nat_HF-7900 | C4-79 |

| Published name | Original Name |
|---|---|
| C3_Crn-05 | C3_hetC2_HFuse-05, C3_crown-05 |
| C5_Crn-07 | C5_hetC2_HFuse-07, C5_crown-07 |
| C5_Crn_HF-12 | crn_arm-12, C5_crown-07_HFuse-12 |
| C5_Crn_HF-26 | crn_arm-26, C5_crown-07_HFuse-26 |
| C5_Crn_HF-12_26 | crn_arm-12_26 |

| Published name | Original Name |
|---|---|
| D2_Wm-01 | D2-1 |
| D2_Wm-01_trunc | D2-1_trunc |
| D2_Wm-02 | D2-2 |
| T_Wm-1606 | T16.6 |
| 132_Wm-42 | w2c_DHRsp-42 |
| C3_HF_Wm-0024A | w2c_DHRsp-24A_capped |

### Supplementary Main-Text Protein Sequences

HelixDock
>C3_HD-1069
(SEQ ID NO: 2)
MGHHHHHHGGNDEKEKLKELLKRAEELAKSPDPEDLKEAVRLAEEVVRERPGSNLAKKALEIILRAAEELAKLPDPK
ALIAAVLAAIKVVREQPGSNLAKKALEIILRAAEELAKLPDPLALAAAVVAATIVVLIQPGSELAKKALEIIERAAE
ELKKSPDPLAQLLAIAAEALVIALKSSSEETIKEMVKLITLALLTSLLILILILLDLKEMLERLEKNPDKDVIVKVL
KVIVKAIEASVLNQAISAINQILLALSD
HelixFuse
>C3_nat_HF-0005
(SEQ ID NO: 4)
MGHHHHHHGGSSEEEQERIRRILKEARKSGTEESLRQAIEDVAQLAKKSQDSEVLEEAIRVILRIAKESGSEEALRQ
AIRAVAEIAKEAQDSEVLEEAIRVILRIAKESGSEEALRQALRAVAEIAEEAKDERVRKEAVRVMLQIAKESGSKEA
VKLAFEMILRVVRIIAVLRANSVEEAKEKALAVFEGGVLAIEITFTVPDADTVIKELSFLEKEGAIIGAGTVISVEQ
CRKAVESGALFIVSPHLDEEISQFCDEAGVAYAPGVMTPTELVKAMKLGHRILKLFPGEVVGPQFVKAMKGPFPNVR
FVPIGGVNLDNVAEWFKAGVLAVGVGSALVKGTPDEVREKAKAFVEKIKAA
>C4_nat_HF-7900
(SEQ ID NO: 6)
MASSWVMLGLLLSLLNRLSLAAEAYKKAIELDPNDALAWLLLGSVLLLLGREEEAEEAARKAIELKPEMDSARRLEG
IIELIRRAREAAERAQEAAERTGDPRVRELARELKRLAQEAAEEVRRDPDSKDVNEALKLIVEAIEAAVRALEAAER
TGDPEVRELARELVRLAVEAAEEVQRNPSSSDVNEALKLIVEAIDAAVRALEAAEKTGDPEVRELARELVRLAVEAA
EEVQRNPSSEEVNEALKDIVKAIQEAVESLREAEESGDPEKREKARERVREAVERAEEVQRDPSSGGSWGLEHHHHH
H
>C5_HF-3921
(SEQ ID NO: 8)
MGHHHHHHGSGSENLYFQGGSSDLQEVADRIVEQLKREGRSPEEARKEARRLIEEIKQSAGGDSELIEVAVRIVKEL
EEQGRSPSEAAKEAVELIERIRRAAGGDSELIEVAVRIVKELEEQGRSPSEAAKEAVELIERIRRAAGGDSELIEVA
VRIVKELEEQGRSPSEAAKEAVELIERIRRAAGGDSELIEVAVRIVKELEEQGRSPSEAAKEAVELIERIRRAAGGD
SELIEVAVRIVKFLEEAGMSPSEAAKVAVELIERIRRAAGGDSELIEKAVRIVRRLERRGLSPAEAAKIAVAIIAAE
VLSREAEKIREETEEVKKEIEESKKRPQSESAKNLILIMQLLINQIRLLALQIQMLRLQLEL
>C5_HF-2101
(SEQ ID NO: 10)
MGHHHHHHGSGSENLYFQGGSSEKEKVEELAQRIREQLPDTELAREAQELADEARKSDDSEALKVVYLALRIVQQLP
DTELAREALELAKEAVKSTDSEALKVVYLALRIVQQLPDTELAREALELAKEAVKSTDSEALKVVYLALRIVQQLPD
TELAREALELAKEAVKSTDSEALKVVYLALRIVQQLPDTELAREALELAKEAVKSTDSEALKVVYLALRIVQQLPDT
ELAREALELAKEAVKSTDSEALKVVYLALRIVQLLPDTDLARKALELAKEAVKMDDQEVLKVVYKALQIVADKPNTE
EADEALRDARLKLEAARLRREMEKIREETEEVKKEIEESKKRPQSESAKNLILIMQLLINQIRLLALQIRMLDLQLK
L
>C5_HF-0019
(SEQ ID NO: 12)
MGHHHHHHGGSENLYFQSGGNDEKEKLKELLKRAEELAKSPDPEDLKEAVRLAEEVVRERPGSNLAKKALEIILRAA
EELAKLPDPEALKEAVKAAEKVVREQPGSNLAKKAQEIILRAAEELAKLEDEEALKEAIKAAEKVIELEPGSELAKE
AKRIIEKAAKMLADILRKEMEKIREETEEVKKEIEESKKRPQSESAKNLILIMQLLINQIRLLALQIRMLVLQLIL
>C6_HF-0075
(SEQ ID NO: 14)
MGHHHHHHGWSGSIQEKAKQSVIRKVKEEGGSEEEARERAKEVEERLKKEADDSTLVRAAAAVVLYVLEKGGSTEEA
VQRAREVIERLKKEASDSTLVRAAAAVVLYVLEKGGSTEEAVQRAREVIERLKKEASDSTLVRAAAAVVLYVLEKGG
STEEAVQRAREVIERLKKEASDSTLVRAAAAVVLYVLEKGGSTEEAVQRAREVIERLKKEASDSTLVRAAAAVVLYV
LEKGGSTEEAVQRAREVIERLKKEASDSTLVRAAAAVVLYVLEKGGSTEEAVDRAREVIEALKKFANDEEEIRRAAK
VVLKVLETGGSVEEAMIRAALEILLDMLKEAAKKLKKLEDKTRRSEEISKTDDDPKAQSLQLIAESLMLIAESLLII
AISLLLSSLAG
>C6_HF-0080
(SEQ ID NO: 16)
MGHHHHHHGWSGSTKEKARQLAEEAKETAEKVGDPELIKLAEQASQEGDSEKAKAILLAAEAARVAKEVGDPELIKL
ALEAARRGDSEKAKAILLAAEAARVAKEVGDPELIKLALEAARRGDSEKAKAILLAAEAARVAKEVGDPELIKLALE
AARRGDSEKAKAILLAAEAARVAKEVGDPELIKLALEAARRGDSEKAKAILLAAEAARVAKEVGDPELIKLALEAAR
RGDSEKAKAILLAAEAARVAKEAGIPEMIKAALRAARLGASDAAQAILEAADEARKAREEGDKKKEKSAELKALLAL
AKVKLKRLEDKIRRSEEISKTDDDPKAQSLQLIAESLMLIAESLLIIAISLLLSSDAG
Crowns
>C3_Crn-05
(SEQ ID NO: 18)
MGDRSDHAKKLKTFLENLRRHLDRLDKHIKQLRDILSENPEDERVKDVIDLSERSVRIVKIVIKIFEDSVRKLLKQI
NKEAEELAKSPDPEDLKRAVELAEAVVRADPGSNLSKKALEIILRAAAELAKLPDPDALAAAARAASKVQQEQPGSN
LAKAAQEIMRQASRAAEEAARRAKETLEKAEKDGDPETALKAVETVVKVARALNQIATMAGSEEAQERAARVASEAA
RLAERVLELAEKQGDPEVARRARELQEKVLDILLDILEQILQTATKIIDDANKLLEKLRRSERKDPKVVETYVELLK
RHERLVKQLLEIAKAHAEAVEGGSLEHHHHHH
>C5_Crn-07
(SEQ ID NO: 20)
MGDRSEHAKKLKTFLENLRRHLDRLDKHIKQLRDILSENPEDERVKDVIDLSERSVRIVKIVIKIFEDSVRKLEKQI
LKEAEELAKSPDPEDLKRAVELARAVIEANPGSNLSRKAMEIIERAARELSKLPDPEAQRTAIEAASQLATMAAATG
NTDQVRRAAELMKEIARLAGTEEAKDLALDALLDVLETALQIATKIIDDANKLLEKLRRSERKDPKVVETYVELLKR
HEEAVRLLLEVAKTHADIVEGGSLEHHHHHH
>C5_Crn_HF-12
(SEQ ID NO: 22)
MGDRSEHAKKLKTFLENLRRHLDRLDKHIKQLRDILSEHPHDERVKDVIDLSERSVRIVKKVIKIFEDSVRELEKMI
LKEAEELAKSPDPEDLKRAVELARAVIEANPGSNLSRKAMEIIERAARELSKLPDPEAQRTAIEAASQLATMAAATG
NTDQVRRAAKLMMRIAILAGTEEASDLALDALLDVLETALQIATKIIDDANKLLEKLRRSHHHDPKVVETYVELLKR
HEEAVRLLLDVAIMHALIVVMQDAIEAAREGDKDRARKALQDALELARLAGTTEAVEAALLVVEAVAVAAARAGATD
VVREALEVALEIARESGTTEAVKLALEVVASVAIEAARRGNTDAVREALEVALEIARESGTEEAVRLALEVVKRVSD
EAKKQGNEDAVKEAEEVRKKIEEES
>C5_Crn_HF-26
(SEQ ID NO: 24)
MGTESKVLEAEMSIKKAEWSAREGNPEKATEDLMRAMLLIRELDVLAQKTGSAEVLVKAAALAEKLAKVAREVGDPE
MAREAEKLARALAAKLLSMHAKLLATFLENLRRHLDRLDKHIKQLRDILSEHPHDERVKDVIDLSERSVRIVKIVIK
IFEDSVRKLLKEMLKRAEELAKSPDPLDLKAAVDVARAVIEANPGSNLSRKAMEIIERAARELSKLPDPLAIATAIE
AASQLATMAAAIGNIDQVRRAAELMKEIARLAGIDLAKAAALLALLRVLETALQIATKIIDDANKLLEKLRRSHHHD
PKVVETYVELLKRHEEAVRLLLEVAKTHADIVE
>C5_Crn_HF-12_26
(SEQ ID NO: 26)
MGTESKVLEAEMSIKKAEWSAREGNPEKATEDLMRAMLLIRELDVLAQKTGSAEVLVKAAALAEKLAKVAREVGDPE
MAREAEKLARALAAKLLSMHAKLLATFLENLRRHLDRLDKHIKQLRDILSEHPHDERVKDVIDLSERSVRIVKKVIK
IFEDSVRELLKMMLKRAEELAKSPDPEDLKAAVDVARAVIEANPGSNLSRKAMEIIERAARELSKLPDPEAIATAIE
AASQLATMAAAIGNIDQVRRAAKLMMRIAILAGIDLASAAALDALLRVLETALQIATKIIDDANKLLEKLRRSHHHD
PKVVETYVELLKRHEEAVRLLLDVAIMHALIVVMQDAIEAAREGDKDRARKALQDALELARLAGTTEAVEAALLVVE
AVAVAAARAGATDVVREALEVALEIARESGTTEAVKLALEVVASVAIEAARRGNIDAVREALEVALEIARESGTEEA
VRLALEVVKRVSDEAKKQGNEDAVKEAEEVRKKIEEES
Dihedral rings
>D2_Wm-01A
(SEQ ID NO: 28)
MGTREESLKEQLRSLREQAELAARLLRLLKELERLQREGSSDEDVRELLREIKELVAEIIKLIMEQLLLIAEQLLGR
SEAAELALRAIRLALELCRQSIDLEECLRLLKTAIKALENALRHPDSTTAKARLMAITARLLAQQLRIQHPDSQAAR
DAEKLADQAERAVRLATRLYEEHPNAEISEMCSQAAYAAALMASIAAILAQRHPDSQIARDLIRLASELAEMVKRMC
ERGGSWGLEHHHHHH
>D2_Wm-01B
(SEQ ID NO: 30)
MGTREELAKELLRSLREQAESLARQLRLLKELERLQREGSSDEDVRELLREIKELAAEQIKLIMEQLLLIAELTLGR
SEAAELALDAIRQALEACRTMDNQEACTRLLKLAIQMLELATRAPDAEAAKLALEAAKKAIELANRHPGSQAAEDAT
KLAQQAMEAVRLALKLYEEHPNADIADLCRRAAAEAAEAASKAAELAQRHPDSQAARDAIKLASQAAEAVKLACELA
QEHPNADKAKLCILLASAAALLASIAAMLAQRHPDSQEARDMIRIASELAELVKEICER
>D2_Wm-02A
(SEQ ID NO: 32)
MGTREEIIRELARSLAEQAELTARLERSLREQERLQREGSSDEDVRELIREQKELVREILKLIAEQILLIAELLLAS
TRSEAAELALRAIRNAIEACKNADNEEMCRQLMRMAQNALELATQAPDAEAAKAALRAIDLAVELASRHPGSQAADD
ALKLAQQAAEAVKLALDLYREHPNADIADLCRKAAKEAAEAASKAAELAQRHPDSQAARDAIKLASQAAEAVKLACE
LAQEHPNAEIAKMCILAASAAALMASIAAILAQRHPDSQIARDLIRLASELAEMVKRMCERGGSWGLEHHHHHH
>D2_Wm-02B
(SEQ ID NO: 34)
MGTREELAKELLRSLREQAESLARQLRLLKELERLQREGSSDEDVRELLREIKELAAEQIKLIMEQLLLIAELMLGR
SEAAELALEAIRLALELCRQSTDQEQCIDLLRQATEALETATRYPDDINAKAKLMAITARLLAQQLRIQHPDSQAAR
DAEKLADQAEKAVRLAKRLYEEHPNADKSELCSQLAYAAALLASIAAMLAQRHPDSQEARDMIRIASELAELVKEIC
ER
>D2_Wm-01_truncA
(SEQ ID NO: 36)
MGTREESLKEQLRSLREQAELAARLLRLQREGSSDEDVKELVAEIIKLIMEQLLLIAEQLLGRSEAAELALRAIRLA
LELCRQSIDLEECLRLLKTAIKALENALRHPDSTTAKARLMAITARLLAQQLRIQHPDSQAARDAEKLADQAERAVR
LATRLYEEHPNAEISEMCSQAAYAAALMASIAAILAQRHPDSQIARDLIRLASELAEMVKRMCERGGSWGLEHHHHH
H
>D2_Wm-01_truncB
(SEQ ID NO: 38)
MGTREELAKELLRSLREQAESLARQLRLQREGSSDEDVKELAAEQIKLIMEQLLLIAELTLGRSEAAELALDAIRQA
LEACRTMDNQEACTRLLKLAIQMLELATRAPDAEAAKLALEAAKKAIELANRHPGSQAAEDATKLAQQAMEAVRLAL
KLYEEHPNADIADLCRRAAAEAAEAASKAAELAQRHPDSQAARDAIKLASQAAEAVKLACELAQEHPNADKAKLCIL
LASAAALLASIAAMLAQRHPDSQEARDMIRIASELAELVKEICER
Point Group nanocage
>T_Wm-1606
(SEQ ID NO: 40
MGDEEKKKELLKQLEDSLIELIRILAELKEMLERLEKNPDKDTIVKVLKVIVKAIEASVANQAISAMNQGADANAKD
SDGRTPLHHAAEAGAAAVVKVAIDAGADVNEKDSDGRTPLHHAAENGHAEVVTLLIEKGADVNEKDSDGRTPLHHAA
ENGHDEVVLILLLKGADVNAKDSDGRTPLHHAAENGHKRVVLVLILAGADVNTSDSDGRTPLDLAREHGNEEVVKAL
EKQGGWLEHHHHHH
>I32_Wm-42A
(SEQ ID NO: 42)
MGGSELEIVIRLQILNLELARKLLEAVARLQELNIDLVRKTSELTDEKTIREEIRKVKEESKRIVKEAEDEIKKAAL
ISADLAAKAIKRAIDRAKKLLEKGEKEDAEDVLREARSAIRLVTELLERIAKNSSTPEEALRAAELLVRLIILLIKI
AALLAAAGNKEEADKVLDEAKELIERVRELLEKISKNSDTPELSKRAKELELILRLADLAIKAMKNIGSDEARQAVK
EMARLAKEALEMGMSEAAKAAIELLELLAEAFAGSDVASLAVKAIAKIAETALRNGS
*bolded Ser residue denotes additional mutation of Cys to remove a disulfide
bond at the interface termini
>I32_Wm-42B
(SEQ ID NO: 44)
MGSDTAKEAIQRLEDLARKYSGSDVASLAVKAIEKIARTAVENGSEETAEEAEKRLRELAEDYQGSNVASLAASAIA
EIAAARARFAAREMGDPRVEEIAKELERLAKEAAERVERRPDSEEDYRKLELAALIIKLFVSLLKQKRLAERLKELL
RELERLQREGSSDEDVRELLREIKELVEEIEKLARKQEYLVTELAKMMGGSGGSGGSGGSLEHHHHHH
*bolded Ser residue denotes additional mutation of Cys to remove a disulfide
bond at the interface termini
>C3_HF_Wm_0024A
(SEQ ID NO: 46)
MGKELEIVARLQQLNIELARKLLEAVARLQELNIDLVRKTSELTDEKTIREEIRKVKEESKRIVEEAEQEIRKAEAE
SLRLTAEAAADAARKAALRMGDERVRRLAAELVRLAQEAAEEATRDPNSSDQNEALRLIILAIEAAVRALDKAIEKG
DPEDRERAREMVRAAVRAAELVQRYPSASAANEALKALVAAIDEGDKDAARCAEELVEQAEEALRKKNPEEARAVYE
AARDVLEALQRLEEAKRRGDEEERREAEERLRQACERARKKNGGSLEHHHHHH

*Underline denotes added linker, start codon, and his-tag residues used for Ni-NTA purification.

### REFERENCES

1. Chen, Z. et al. De novo design of protein logic gates. Science 368, 78-84 (2020).
2. Dyer, K. N. et al. High-Throughput SAXS for the Characterization of Biomolecules in Solution: A Practical Approach. in Structural Genomics (ed. Chen, Y. W.) vol. 1091 245-258 (Humana Press, 2014).
3. Schneidman-Duhovny, D., Hammel, M. & Sali, A. FoXS: a web server for rapid computation and fitting of SAXS profiles. Nucleic Acids Res. 38, W540-W544 (2010).
4. Kabsch, W. XDS. Acta Crystallogr. D Biol. Crystallogr. 66, 125-132 (2010).
5. Winn, M. D. et al. Overview of the CCP 4 suite and current developments. Acta Crystallogr. D Biol. Crystallogr. 67, 235-242 (2011).
6. McCoy, A. J. et al. Phaser crystallographic software. J. Appl. Crystallogr. 40, 658-674 (2007).
7. Adams, P. D. et al. PHENIX: a comprehensive Python-based system for macromolecular structure solution. Acta Crystallogr. D Biol. Crystallogr. 66, 213-221 (2010).
8. Emsley, P. & Cowtan, K. Coot: model-building tools for molecular graphics. Acta Crystallogr. D Biol. Crystallogr. 60, 2126-2132 (2004).
9. Williams, C. J. et al. MolProbity: More and better reference data for improved all-atom structure validation: PROTEIN SCIENCE.ORG. Protein Sci. 27, 293-315 (2018).
10. Zivanov, J. et al. New tools for automated high-resolution cryo-EM structure determination in RELION-3. eLife 7, e42166 (2018).
11. Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
12. Bell, J. M., Chen, M., Durmaz, T., Fluty, A. C. & Ludtke, S. J. New software tools in EMAN2 inspired by EMDatabank map challenge. J. Struct. Biol. 204, 283-290 (2018).
13. Hohn, M. et al. SPARX, a new environment for Cryo-EM image processing. J. Struct. Biol. 157, 47-55 (2007).
14. Suloway, C. et al. Automated molecular microscopy: The new Leginon system. J. Struct. Biol. 151, 41-60 (2005).
15. Lander, G. C. et al. Appion: an integrated, database-driven pipeline to facilitate EM image processing. J. Struct. Biol. 166, 95-102 (2009).
16. Zheng, S. Q. et al. MotionCor2: anisotropic correction of beam-induced motion for improved cryo-electron microscopy. Nat. Methods 14, 331-332 (2017).
17. Tegunov, D. & Cramer, P. Real-time cryo-electron microscopy data preprocessing with Warp. Nat. Methods 16, 1146-1152 (2019).
18. Bepler, T. et al. Positive-unlabeled convolutional neural networks for particle picking in cryo-electron micrographs. Nat. Methods 16, 1153-1160 (2019).
19. Tan, Y. Z. et al. Addressing preferred specimen orientation in single-particle cryo-EM through tilting. Nat. Methods 14, 793-796 (2017).
20. Pettersen, E. F. et al. UCSF Chimera—a visualization system for exploratory research and analysis. J. Comput. Chem. 25, 1605-1612 (2004).
21. Emsley, P., Lohkamp, B., Scott, W. G. & Cowtan, K. Features and development of Coot. Acta Crystallogr. D Biol. Crystallogr. 66, 486-501 (2010).
22. Echols, N. et al. Graphical tools for macromolecular crystallography in PHENIX. J. Appl. Crystallogr. 45, 581-586 (2012).
23. Brunette, T. et al. Exploring the repeat protein universe through computational protein design. Nature 528, 580-584 (2015).
24. Brunette, T. et al. Modular repeat protein sculpting using rigid helical junctions. Proc. Natl. Acad. Sci. 117, 8870-8875 (2020).
25. Brunette, T. et al. Exploring the repeat protein universe through computational protein design. Nature 528, 580-584 (2015).
26. Geiger-Schuller, K. et al. Extreme stability in de novo-designed repeat arrays is determined by unusually stable short-range interactions. Proc. Natl. Acad. Sci. 115, 7539-7544 (2018).

We claim:

1. A polypeptide comprising an amino acid sequence at least 50% identical to the amino acid sequence selected from the group consisting of SEQ ID NOS: 1-46, wherein residues in parentheses are optional.

2. The polypeptide of claim 1, comprising an amino acid sequence at least 75% identical to the amino acid sequence selected from the group consisting of SEQ ID NOS:1-46, wherein residues in parentheses are optional.

3. The polypeptide of claim 1, comprising an amino acid sequence at least 90% identical to the amino acid sequence selected from the group consisting of SEQ ID NOS:1-46, wherein residues in parentheses are optional.

4. The polypeptide of claim 1, wherein amino acid changes from the reference polypeptide of any one of SEQ ID NOS:1-46 are conservative amino acid substitutions

5. The polypeptide of claim 1, wherein at least 1 or more of the non-polar residues in bold font as shown in Table 1 are invariant relative to the reference polypeptide.

6. The polypeptide of claim 1, wherein at least 10 or more of the non-polar residues in bold font as shown in Table 1 are invariant relative to the reference polypeptide.

7. The polypeptide of claim 1, wherein at least 1 or more of the residues in bold font as shown in Table 1 are invariant relative to the reference polypeptide.

8. The polypeptide of claim 1, wherein at least 10 or more of the residues in bold font as shown in Table 1 are invariant relative to the reference polypeptide.

9. The polypeptide of claim 1, further comprising an additional functional domain fused to the polypeptide.

10. A nucleic acid encoding the polypeptide of claim 1.

11. An expression vector comprising the nucleic acid of claim 10 operatively linked to a suitable control sequence.

12. A host cell comprising the expression vector of claim 11.

13. An oligomer, comprising two or more polypeptides according to claim 1.

14. The oligomer of claim 15, wherein the oligomer comprises a homo-oligomer comprising two or more identical polypeptides comprising an amino acid sequence at least 50% identical to the amino acid sequence selected from the group consisting of SEQ ID NOS: 1-26, 39-40, and 45-46.

15. The oligomer of claim 13, wherein the oligomer comprises a hetero-oligomer, wherein the hetero-oligomer comprises two different polypeptides comprising an amino acid sequence at least 50% identical to the amino acid sequence selected from the group consisting of:
SEQ ID NO: 27 and 29;
SEQ ID NO:27 and 30;
SEQ ID NO:28 and 29;
SEQ ID NO:28 and 30;
SEQ ID NO: 31 and 33;
SEQ ID NO:31 and 34;
SEQ ID NO:32 and 33;
SEQ ID NO:32 and 34;
SEQ ID NO: 35 and 37;
SEQ ID NO:35 and 38;
SEQ ID NO:36 and 37;
SEQ ID NO:36 and 38;
SEQ ID NO: 41 and 43;
SEQ ID NO:41 and 44;
SEQ ID NO:42 and 43; and
SEQ ID NO:42 and 44.

16. The oligomer of claim 13, wherein the oligomer comprises a two-component dihedral assembly, wherein the two component dihedral assembly comprises two different polypeptide comprising an amino acid sequence at least 50% identical to the amino acid sequence selected from the group consisting of:
SEQ ID NO: 27 and 29;
SEQ ID NO:27 and 30;
SEQ ID NO:28 and 29;
SEQ ID NO:28 and 30;
SEQ ID NO: 31 and 33;
SEQ ID NO:31 and 34;
SEQ ID NO:32 and 33;
SEQ ID NO:32 and 34;
SEQ ID NO: 35 and 37;
SEQ ID NO:35 and 38;
SEQ ID NO:36 and 37; and
SEQ ID NO:36 and 38.

17. The oligomer of claim 13, wherein the oligomer comprises a one-component tetrahedral protein cage, wherein the one-component tetrahedral protein cage comprises an amino acid sequence at least 50% identical to the amino acid sequence of SEQ ID NO:39 or 40.

18. The oligomer of claim 13, wherein the oligomer comprises a two-component icosahedral protein cage, wherein the two icosahedral protein cage comprises two different polypeptide comprising an amino acid sequence at least 50% identical to the amino acid sequence selected from the group consisting of:
SEQ ID NO: 41 and 43;
SEQ ID NO:41 and 44;
SEQ ID NO:42 and 43; and
SEQ ID NO:42 and 44.

19. A composition comprising the oligomer of claim 13 and a therapeutic moiety or diagnostic moiety covalently attached to the oligomer.

20. A method for designing multi-scale protein complexes by combinatorial assembly of oligomeric helical bundle and repeat protein building blocks, comprising any methods described herein.


SEQUENCE LISTING (46 sequences)

SEQ ID NO: 1 - protein, 249 aa, Artificial Sequence
  Synthetic
NDEKEKLKELLKRAEELAKSPDPEDLKEAVRLAEEVVRERPGSNLAKKALEIILRAAEELAKLPDPKALIAAVLAAIKVVREQPGSNLAKKALEIILRAAEELAKLPDPLALAAAVVAATIVVLTQPGSELAKKALEIIERAAEELKKSPDPLAQLLAIAAEALVIALKSSSEETIKEMVKLTTLALLTSLLILILILLDLKEMLERLEKNPDKDVIVKVLKVIVKAIEASVLNQAISAINQILLALSD

SEQ ID NO: 2 - protein, 259 aa, Artificial Sequence
  Synthetic
  MISC_FEATURE (1)..(10): optionally absent
MGHHHHHHGGNDEKEKLKELLKRAEELAKSPDPEDLKEAVRLAEEVVRERPGSNLAKKALEIILRAAEELAKLPDPKALIAAVLAAIKVVREQPGSNLAKKALEIILRAAEELAKLPDPLALAAAVVAATIVVLTQPGSELAKKALEIIERAAEELKKSPDPLAQLLAIAAEALVIALKSSSEETIKEMVKLTTLALLTSLLILILILLDLKEMLERLEKNPDKDVIVKVLKVIVKAIEASVLNQAISAINQILLALSD

SEQ ID NO: 3 - protein, 348 aa, Artificial Sequence
  Synthetic
SEEEQERIRRILKEARKSGTEESLRQAIEDVAQLAKKSQDSEVLEEAIRVILRIAKESGSEEALRQAIRAVAEIAKEAQDSEVLEEAIRVILRIAKESGSEEALRQALRAVAEIAEEAKDERVRKEAVRVMLQIAKESGSKEAVKLAFEMILRVVRIIAVLRANSVEEAKEKALAVFEGGVLAIEITFTVPDADTVIKELSFLEKEGAIIGAGTVTSVEQCRKAVESGALFIVSPHLDEEISQFCDEAGVAYAPGVMTPTELVKAMKLGHRILKLFPGEVVGPQFVKAMKGPFPNVRFVPTGGVNLDNVAEWFKAGVLAVGVGSALVKGTPDEVREKAKAFVEKIKAA

SEQ ID NO: 4 - protein, 359 aa, Artificial Sequence
  Synthetic
  MISC_FEATURE (1)..(11): optionally absent
MGHHHHHHGGSSEEEQERIRRILKEARKSGTEESLRQAIEDVAQLAKKSQDSEVLEEAIRVILRIAKESGSEEALRQAIRAVAEIAKEAQDSEVLEEAIRVILRIAKESGSEEALRQALRAVAEIAEEAKDERVRKEAVRVMLQIAKESGSKEAVKLAFEMILRVVRIIAVLRANSVEEAKEKALAVFEGGVLAIEITFTVPDADTVIKELSFLEKEGAIIGAGTVTSVEQCRKAVESGALFIVSPHLDEEISQFCDEAGVAYAPGVMTPTELVKAMKLGHRILKLFPGEVVGPQFVKAMKGPFPNVRFVPTGGVNLDNVAEWFKAGVLAVGVGSALVKGTPDEVREKAKAFVEKIKAA

SEQ ID NO: 5 - protein, 295 aa, Artificial Sequence
  Synthetic
ASSWVMLGLLLSLLNRLSLAAEAYKKAIELDPNDALAWLLLGSVLLLLGREEEAEEAARKAIELKPEMDSARRLEGIIELIRRAREAAERAQEAAERTGDPRVRELARELKRLAQEAAEEVRRDPDSKDVNEALKLIVEAIEAAVRALEAAERTGDPEVRELARELVRLAVEAAEEVQRNPSSSDVNEALKLIVEAIDAAVRALEAAEKTGDPEVRELARELVRLAVEAAEEVQRNPSSEEVNEALKDIVKAIQEAVESLREAEESGDPEKREKARERVREAVERAEEVQRDPSS

SEQ ID NO: 6 - protein, 309 aa, Artificial Sequence
  Synthetic
  MISC_FEATURE (1)..(1): optionally absent
  MISC_FEATURE (297)..(309): optionally absent
MASSWVMLGLLLSLLNRLSLAAEAYKKAIELDPNDALAWLLLGSVLLLLGREEEAEEAARKAIELKPEMDSARRLEGIIELIRRAREAAERAQEAAERTGDPRVRELARELKRLAQEAAEEVRRDPDSKDVNEALKLIVEAIEAAVRALEAAERTGDPEVRELARELVRLAVEAAEEVQRNPSSSDVNEALKLIVEAIDAAVRALEAAEKTGDPEVRELARELVRLAVEAAEEVQRNPSSEEVNEALKDIVKAIQEAVESLREAEESGDPEKREKARERVREAVERAEEVQRDPSSGGSWGLEHHHHHH

SEQ ID NO: 7 - protein, 349 aa, Artificial Sequence
  Synthetic
SDLQEVADRIVEQLKREGRSPEEARKEARRLIEEIKQSAGGDSELIEVAVRIVKELEEQGRSPSEAAKEAVELIERIRRAAGGDSELIEVAVRIVKELEEQGRSPSEAAKEAVELIERIRRAAGGDSELIEVAVRIVKELEEQGRSPSEAAKEAVELIERIRRAAGGDSELIEVAVRIVKELEEQGRSPSEAAKEAVELIERIRRAAGGDSELIEVAVRIVKFLEEAGMSPSEAAKVAVELIERIRRAAGGDSELIEKAVRIVRRLERRGLSPAEAAKIAVAIIAAEVLSREAEKIREETEEVKKEIEESKKRPQSESAKNLILIMQLLINQIRLLALQIQMLRLQLEL

SEQ ID NO: 8 - protein, 370 aa, Artificial Sequence
  Synthetic
  MISC_FEATURE (1)..(21): optionally absent
MGHHHHHHGSGSENLYFQGGSSDLQEVADRIVEQLKREGRSPEEARKEARRLIEEIKQSAGGDSELIEVAVRIVKELEEQGRSPSEAAKEAVELIERIRRAAGGDSELIEVAVRIVKELEEQGRSPSEAAKEAVELIERIRRAAGGDSELIEVAVRIVKELEEQGRSPSEAAKEAVELIERIRRAAGGDSELIEVAVRIVKELEEQGRSPSEAAKEAVELIERIRRAAGGDSELIEVAVRIVKFLEEAGMSPSEAAKVAVELIERIRRAAGGDSELIEKAVRIVRRLERRGLSPAEAAKIAVAIIAAEVLSREAEKIREETEEVKKEIEESKKRPQSESAKNLILIMQLLINQIRLLALQIQMLRLQLEL

SEQ ID NO: 9 - protein, 365 aa, Artificial Sequence
  Synthetic
SEKEKVEELAQRIREQLPDTELAREAQELADEARKSDDSEALKVVYLALRIVQQLPDTELAREALELAKEAVKSTDSEALKVVYLALRIVQQLPDTELAREALELAKEAVKSTDSEALKVVYLALRIVQQLPDTELAREALELAKEAVKSTDSEALKVVYLALRIVQQLPDTELAREALELAKEAVKSTDSEALKVVYLALRIVQQLPDTELAREALELAKEAVKSTDSEALKVVYLALRIVQLLPDTDLARKALELAKEAVKMDDQEVLKVVYKALQIVADKPNTEEADEALRDARLKLEAARLRREMEKIREETEEVKKEIEESKKRPQSESAKNLILIMQLLINQIRLLALQIRMLDLQLKL

SEQ ID NO: 10 - protein, 386 aa, Artificial Sequence
  Synthetic
  MISC_FEATURE (1)..(21): optionally absent
MGHHHHHHGSGSENLYFQGGSSEKEKVEELAQRIREQLPDTELAREAQELADEARKSDDSEALKVVYLALRIVQQLPDTELAREALELAKEAVKSTDSEALKVVYLALRIVQQLPDTELAREALELAKEAVKSTDSEALKVVYLALRIVQQLPDTELAREALELAKEAVKSTDSEALKVVYLALRIVQQLPDTELAREALELAKEAVKSTDSEALKVVYLALRIVQQLPDTELAREALELAKEAVKSTDSEALKVVYLALRIVQLLPDTDLARKALELAKEAVKMDDQEVLKVVYKALQIVADKPNTEEADEALRDARLKLEAARLRREMEKIREETEEVKKEIEESKKRPQSESAKNLILIMQLLINQIRLLALQIRMLDLQLKL

SEQ ID NO: 11 - protein, 210 aa, Artificial Sequence
  Synthetic
NDEKEKLKELLKRAEELAKSPDPEDLKEAVRLAEEVVRERPGSNLAKKALEIILRAAEELAKLPDPEALKEAVKAAEKVVREQPGSNLAKKAQEIILRAAEELAKLEDEEALKEAIKAAEKVIELEPGSELAKEAKRIIEKAAKMLADILRKEMEKIREETEEVKKEIEESKKRPQSESAKNLILIMQLLINQIRLLALQIRMLVLQLIL

SEQ ID NO: 12 - protein, 230 aa, Artificial Sequence
  Synthetic
  MISC_FEATURE (1)..(20): optionally absent
MGHHHHHHGGSENLYFQSGGNDEKEKLKELLKRAEELAKSPDPEDLKEAVRLAEEVVRERPGSNLAKKALEIILRAAEELAKLPDPEALKEAVKAAEKVVREQPGSNLAKKAQEIILRAAEELAKLEDEEALKEAIKAAEKVIELEPGSELAKEAKRIIEKAAKMLADILRKEMEKIREETEEVKKEIEESKKRPQSESAKNLILIMQLLINQIRLLALQIRMLVLQLIL

SEQ ID NO: 13 - protein, 384 aa, Artificial Sequence
  Synthetic
SIQEKAKQSVIRKVKEEGGSEEEARERAKEVEERLKKEADDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVDRAREVIEALKKFANDEEEIRRAAKVVLKVLETGGSVEEAMIRAALEILLDMLKEAAKKLKKLEDKTRRSEEISKTDDDPKAQSLQLIAESLMLIAESLLIIAISLLLSSLAG

SEQ ID NO: 14 - protein, 396 aa, Artificial Sequence
  Synthetic
  MISC_FEATURE (1)..(12): optionally absent
MGHHHHHHGWSGSIQEKAKQSVIRKVKEEGGSEEEARERAKEVEERLKKEADDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVQRAREVIERLKKEASDSTLVRAAAAVVLYVLEKGGSTEEAVDRAREVIEALKKFANDEEEIRRAAKVVLKVLETGGSVEEAMIRAALEILLDMLKEAAKKLKKLEDKTRRSEEISKTDDDPKAQSLQLIAESLMLIAESLLIIAISLLLSSLAG

SEQ ID NO: 15 - protein, 354 aa, Artificial Sequence
  Synthetic
STKEKARQLAEEAKETAEKVGDPELIKLAEQASQEGDSEKAKAILLAAEAARVAKEVGDPELIKLALEAARRGDSEKAKAILLAAEAARVAKEVGDPELIKLALEAARRGDSEKAKAILLAAEAARVAKEVGDPELIKLALEAARRGDSEKAKAILLAAEAARVAKEVGDPELIKLALEAARRGDSEKAKAILLAAEAARVAKEVGDPELIKLALEAARRGDSEKAKAILLAAEAARVAKEAGIPEMIKAALRAARLGASDAAQAILEAADEARKAREEGDKKKEKSAELKALLALAKVKLKRLEDKTRRSEEISKTDDDPKAQSLQLIAESLMLIAESLLIIAISLLLSSDAG

SEQ ID NO: 16 - protein, 366 aa, Artificial Sequence
  Synthetic
  MISC_FEATURE (1)..(12): optionally absent
MGHHHHHHGWSGSTKEKARQLAEEAKETAEKVGDPELIKLAEQASQEGDSEKAKAILLAAEAARVAKEVGDPELIKLALEAARRGDSEKAKAILLAAEAARVAKEVGDPELIKLALEAARRGDSEKAKAILLAAEAARVAKEVGDPELIKLALEAARRGDSEKAKAILLAAEAARVAKEVGDPELIKLALEAARRGDSEKAKAILLAAEAARVAKEVGDPELIKLALEAARRGDSEKAKAILLAAEAARVAKEAGIPEMIKAALRAARLGASDAAQAILEAADEARKAREEGDKKKEKSAELKALLALAKVKLKRLEDKTRRSEEISKTDDDPKAQSLQLIAESLMLIAESLLIIAISLLLSSDAG

SEQ ID NO: 17 - protein, 328 aa, Artificial Sequence
  Synthetic
GDRSDHAKKLKTFLENLRRHLDRLDKHIKQLRDILSENPEDERVKDVIDLSERSVRIVKTVIKIFEDSVRKLLKQINKEAEELAKSPDPEDLKRAVELAEAVVRADPGSNLSKKALEIILRAAAELAKLPDPDALAAAARAASKVQQEQPGSNLAKAAQEIMRQASRAAEEAARRAKETLEKAEKDGDPETALKAVETVVKVARALNQIATMAGSEEAQERAARVASEAARLAERVLELAEKQGDPEVARRARELQEKVLDILLDILEQILQTATKIIDDANKLLEKLRRSERKDPKVVETYVELLKRHERLVKQLLEIAKAHAEAVE

SEQ ID NO: 18 - protein, 340 aa, Artificial Sequence
  Synthetic
  MISC_FEATURE (1)..(1): optionally absent
  MISC_FEATURE (330)..(340): optionally absent
MGDRSDHAKKLKTFLENLRRHLDRLDKHIKQLRDILSENPEDERVKDVIDLSERSVRIVKTVIKIFEDSVRKLLKQINKEAEELAKSPDPEDLKRAVELAEAVVRADPGSNLSKKALEIILRAAAELAKLPDPDALAAAARAASKVQQEQPGSNLAKAAQEIMRQASRAAEEAARRAKETLEKAEKDGDPETALKAVETVVKVARALNQIATMAGSEEAQERAARVASEAARLAERVLELAEKQGDPEVARRARELQEKVLDILLDILEQILQTATKIIDDANKLLEKLRRSERKDPKVVETYVELLKRHERLVKQLLEIAKAHAEAVEGGSLEHHHHHH

SEQ ID NO: 19 - protein, 250 aa, Artificial Sequence
  Synthetic
GDRSEHAKKLKTFLENLRRHLDRLDKHIKQLRDILSENPEDERVKDVIDLSERSVRIVKTVIKIFEDSVRKLEKQILKEAEELAKSPDPEDLKRAVELARAVIEANPGSNLSRKAMEIIERAARELSKLPDPEAQRTAIEAASQLATMAAATGNTDQVRRAAELMKEIARLAGTEEAKDLALDALLDVLETALQIATKIIDDANKLLEKLRRSERKDPKVVETYVELLKRHEEAVRLLLEVAKTHADIVE

SEQ ID NO: 20 - protein, 262 aa, Artificial Sequence
  Synthetic
  MISC_FEATURE (1)..(1): optionally absent
  MISC_FEATURE (252)..(262): optionally absent
MGDRSEHAKKLKTFLENLRRHLDRLDKHIKQLRDILSENPEDERVKDVIDLSERSVRIVKTVIKIFEDSVRKLEKQILKEAEELAKSPDPEDLKRAVELARAVIEANPGSNLSRKAMEIIERAARELSKLPDPEAQRTAIEAASQLATMAAATGNTDQVRRAAELMKEIARLAGTEEAKDLALDALLDVLETALQIATKIIDDANKLLEKLRRSERKDPKVVETYVELLKRHEEAVRLLLEVAKTHADIVEGGSLEHHHHHH

SEQ ID NO: 21 - protein, 409 aa, Artificial Sequence
  Synthetic
GDRSEHAKKLKTFLENLRRHLDRLDKHIKQLRDILSEHPHDERVKDVIDLSERSVRIVKKVIKIFEDSVRELEKMILKEAEELAKSPDPEDLKRAVELARAVIEANPGSNLSRKAMEIIERAARELSKLPDPEAQRTAIEAASQLATMAAATGNTDQVRRAAKLMMRIAILAGTEEASDLALDALLDVLETALQIATKIIDDANKLLEKLRRSHHHDPKVVETYVELLKRHEEAVRLLLDVAIMHALIVVMQDAIEAAREGDKDRARKALQDALELARLAGTTEAVEAALLVVEAVAVAAARAGATDVVREALEVALEIARESGTTEAVKLALEVVASVAIEAARRGNTDAVREALEVALEIARESGTEEAVRLALEVVKRVSDEAKKQGNEDAVKEAEEVRKKIEEES

SEQ ID NO: 22 - protein, 410 aa, Artificial Sequence
  Synthetic
  MISC_FEATURE (1)..(1): optionally absent
MGDRSEHAKKLKTFLENLRRHLDRLDKHIKQLRDILSEHPHDERVKDVIDLSERSVRIVKKVIKIFEDSVRELEKMILKEAEELAKSPDPEDLKRAVELARAVIEANPGSNLSRKAMEIIERAARELSKLPDPEAQRTAIEAASQLATMAAATGNTDQVRRAAKLMMRIAILAGTEEASDLALDALLDVLETALQIATKIIDDANKLLEKLRRSHHHDPKVVETYVELLKRHEEAVRLLLDVAIMHALIVVMQDAIEAAREGDKDRARKALQDALELARLAGTTEAVEAALLVVEAVAVAAARAGATDVVREALEVALEIARESGTTEAVKLALEVVASVAIEAARRGNTDAVREALEVALEIARESGTEEAVRLALEVVKRVSDEAKKQGNEDAVKEAEEVRKKIEEES

SEQ ID NO: 23 - protein, 340 aa, Artificial Sequence
  Synthetic
GTESKVLEAEMSIKKAEWSAREGNPEKATEDLMRAMLLIRELDVLAQKTGSAEVLVKAAALAEKLAKVAREVGDPEMAREAEKLARALAAKLLSMHAKLLATFLENLRRHLDRLDKHIKQLRDILSEHPHDERVKDVIDLSERSVRIVKTVIKIFEDSVRKLLKEMLKRAEELAKSPDPLDLKAAVDVARAVIEANPGSNLSRKAMEIIERAARELSKLPDPLAIATAIEAASQLATMAAATGNTDQVRRAAELMKEIARLAGTDLAKAAALLALLRVLETALQIATKIIDDANKLLEKLRRSHHHDPKVVETYVELLKRHEEAVRLLLEVAKTHADIVE

SEQ ID NO: 24 - protein, 341 aa, Artificial Sequence
  Synthetic
  MISC_FEATURE (1)..(1): optionally absent
MGTESKVLEAEMSIKKAEWSAREGNPEKATEDLMRAMLLIRELDVLAQKTGSAEVLVKAAALAEKLAKVAREVGDPEMAREAEKLARALAAKLLSMHAKLLATFLENLRRHLDRLDKHIKQLRDILSEHPHDERVKDVIDLSERSVRIVKTVIKIFEDSVRKLLKEMLKRAEELAKSPDPLDLKAAVDVARAVIEANPGSNLSRKAMEIIERAARELSKLPDPLAIATAIEAASQLATMAAATGNTDQVRRAAELMKEIARLAGTDLAKAAALLALLRVLETALQIATKIIDDANKLLEKLRRSHHHDPKVVETYVELLKRHEEAVRLLLEVAKTHADIVE

SEQ ID NO: 25 - protein, 499 aa, Artificial Sequence
  Synthetic
GTESKVLEAEMSIKKAEWSAREGNPEKATEDLMRAMLLIRELDVLAQKTGSAEVLVKAAALAEKLAKVAREVGDPEMAREAEKLARALAAKLLSMHAKLLATFLENLRRHLDRLDKHIKQLRDILSEHPHDERVKDVIDLSERSVRIVKKVIKIFEDSVRELLKMMLKRAEELAKSPDPEDLKAAVDVARAVIEANPGSNLSRKAMEIIERAARELSKLPDPEAIATAIEAASQLATMAAATGNTDQVRRAAKLMMRIAILAGTDLASAAALDALLRVLETALQIATKIIDDANKLLEKLRRSHHHDPKVVETYVELLKRHEEAVRLLLDVAIMHALIVVMQDAIEAAREGDKDRARKALQDALELARLAGTTEAVEAALLVVEAVAVAAARAGATDVVREALEVALEIARESGTTEAVKLALEVVASVAIEAARRGNTDAVREALEVALEIARESGTEEAVRLALEVVKRVSDEAKKQGNEDAVKEAEEVRKKIEEES

SEQ ID NO: 26 - protein, 500 aa, Artificial Sequence
  Synthetic
  MISC_FEATURE (1)..(1): optionally absent
MGTESKVLEAEMSIKKAEWSAREGNPEKATEDLMRAMLLIRELDVLAQKTGSAEVLVKAAALAEKLAKVAREVGDPEMAREAEKLARALAAKLLSMHAKLLATFLENLRRHLDRLDKHIKQLRDILSEHPHDERVKDVIDLSERSVRIVKKVIKIFEDSVRELLKMMLKRAEELAKSPDPEDLKAAVDVARAVIEANPGSNLSRKAMEIIERAARELSKLPDPEAIATAIEAASQLATMAAATGNTDQVRRAAKLMMRIAILAGTDLASAAALDALLRVLETALQIATKIIDDANKLLEKLRRSHHHDPKVVETYVELLKRHEEAVRLLLDVAIMHALIVVMQDAIEAAREGDKDRARKALQDALELARLAGTTEAVEAALLVVEAVAVAAARAGATDVVREALEVALEIARESGTTEAVKLALEVVASVAIEAARRGNTDAVREALEVALEIARESGTEEAVRLALEVVKRVSDEAKKQGNEDAVKEAEEVRKKIEEES

SEQ ID NO: 27 - protein, 232 aa, Artificial Sequence
  Synthetic
GTREESLKEQLRSLREQAELAARLLRLLKELERLQREGSSDEDVRELLREIKELVAEIIKLIMEQLLLIAEQLLGRSEAAELALRAIRLALELCRQSTDLEECLRLLKTAIKALENALRHPDSTTAKARLMAITARLLAQQLRTQHPDSQAARDAEKLADQAERAVRLATRLYEEHPNAEISEMCSQAAYAAALMASIAAILAQRHPDSQIARDLIRLASELAEMVKRMCER

SEQ ID NO: 28 - protein, 246 aa, Artificial Sequence
  Synthetic
  MISC_FEATURE (1)..(1): optionally absent
  MISC_FEATURE (234)..(246): optionally absent
MGTREESLKEQLRSLREQAELAARLLRLLKELERLQREGSSDEDVRELLREIKELVAEIIKLIMEQLLLIAEQLLGRSEAAELALRAIRLALELCRQSTDLEECLRLLKTAIKALENALRHPDSTTAKARLMAITARLLAQQLRTQHPDSQAARDAEKLADQAERAVRLATRLYEEHPNAEISEMCSQAAYAAALMASIAAILAQRHPDSQIARDLIRLASELAEMVKRMCERGGSWGLEHHHHHH

SEQ ID NO: 29 - protein, 289 aa, Artificial Sequence
  Synthetic
GTREELAKELLRSLREQAESLARQLRLLKELERLQREGSSDEDVRELLREIKELAAEQIKLIMEQLLLIAELTLGRSEAAELALDAIRQALEACRTMDNQEACTRLLKLAIQMLELATRAPDAEAAKLALEAAKKAIELANRHPGSQAAEDATKLAQQAMEAVRLALKLYEEHPNADIADLCRRAAAEAAEAASKAAELAQRHPDSQAARDAIKLASQAAEAVKLACELAQEHPNADKAKLCILLASAAALLASIAAMLAQRHPDSQEARDMIRIASELAELVKEICER

SEQ ID NO: 30 - protein, 290 aa, Artificial Sequence
  Synthetic
  MISC_FEATURE (1)..(1): optionally absent
MGTREELAKELLRSLREQAESLARQLRLLKELERLQREGSSDEDVRELLREIKELAAEQIKLIMEQLLLIAELTLGRSEAAELALDAIRQALEACRTMDNQEACTRLLKLAIQMLELATRAPDAEAAKLALEAAKKAIELANRHPGSQAAEDATKLAQQAMEAVRLALKLYEEHPNADIADLCRRAAAEAAEAASKAAELAQRHPDSQAARDAIKLASQAAEAVKLACELAQEHPNADKAKLCILLASAAALLASIAAMLAQRHPDSQEARDMIRIASELAELVKEICER

SEQ ID NO: 31 - protein, 291 aa, Artificial Sequence
  Synthetic
GTREEIIRELARSLAEQAELTARLERSLREQERLQREGSSDEDVRELIREQKELVREILKLIAEQILLIAELLLASTRSEAAELALRAIRNAIEACKNADNEEMCRQLMRMAQNALELATQAPDAEAAKAALRAIDLAVELASRHPGSQAADDALKLAQQAAEAVKLALDLYREHPNADIADLCRKAAKEAAEAASKAAELAQRHPDSQAARDAIKLASQAAEAVKLACELAQEHPNAEIAKMCILAASAAALMASIAAILAQRHPDSQIARDLIRLASELAEMVKRMCER

SEQ ID NO: 32 - protein, 305 aa, Artificial Sequence
  Synthetic
  MISC_FEATURE (1)..(1): optionally absent
  MISC_FEATURE (293)..(305): optionally absent
MGTREEIIRELARSLAEQAELTARLERSLREQERLQREGSSDEDVRELIREQKELVREILKLIAEQILLIAELLLASTRSEAAELALRAIRNAIEACKNADNEEMCRQLMRMAQNALELATQAPDAEAAKAALRAIDLAVELASRHPGSQAADDALKLAQQAAEAVKLALDLYREHPNADIADLCRKAAKEAAEAASKAAELAQRHPDSQAARDAIKLASQAAEAVKLACELAQEHPNAEIAKMCILAASAAALMASIAAILAQRHPDSQIARDLIRLASELAEMVKRMCERGGSWGLEHHHHHH

SEQ ID NO: 33 - protein, 232 aa, Artificial Sequence
  Synthetic
GTREELAKELLRSLREQAESLARQLRLLKELERLQREGSSDEDVRELLREIKELAAEQIKLIMEQLLLIAELMLGRSEAAELALEAIRLALELCRQSTDQEQCTDLLRQATEALETATRYPDDTNAKAKLMAITARLLAQQLRTQHPDSQAARDAEKLADQAEKAVRLAKRLYEEHPNADKSELCSQLAYAAALLASIAAMLAQRHPDSQEARDMIRIASELAELVKEICER

SEQ ID NO: 34 - protein, 233 aa, Artificial Sequence
  Synthetic
  MISC_FEATURE (1)..(1): optionally absent
MGTREELAKELLRSLREQAESLARQLRLLKELERLQREGSSDEDVRELLREIKELAAEQIKLIMEQLLLIAELMLGRSEAAELALEAIRLALELCRQSTDQEQCTDLLRQATEALETATRYPDDTNAKAKLMAITARLLAQQLRTQHPDSQAARDAEKLADQAEKAVRLAKRLYEEHPNADKSELCSQLAYAAALLASIAAMLAQRHPDSQEARDMIRIASELAELVKEICER

SEQ ID NO: 35 - protein, 218 aa, Artificial Sequence
  Synthetic
GTREESLKEQLRSLREQAELAARLLRLQREGSSDEDVKELVAEIIKLIMEQLLLIAEQLLGRSEAAELALRAIRLALELCRQSTDLEECLRLLKTAIKALENALRHPDSTTAKARLMAITARLLAQQLRTQHPDSQAARDAEKLADQAERAVRLATRLYEEHPNAEISEMCSQAAYAAALMASIAAILAQRHPDSQIARDLIRLASELAEMVKRMCER

SEQ ID NO: 36 - protein, 232 aa, Artificial Sequence
  Synthetic
  MISC_FEATURE (1)..(1): optionally absent
  MISC_FEATURE (220)..(232): optionally absent
MGTREESLKEQLRSLREQAELAARLLRLQREGSSDEDVKELVAEIIKLIMEQLLLIAEQLLGRSEAAELALRAIRLALELCRQSTDLEECLRLLKTAIKALENALRHPDSTTAKARLMAITARLLAQQLRTQHPDSQAARDAEKLADQAERAVRLATRLYEEHPNAEISEMCSQAAYAAALMASIAAILAQRHPDSQIARDLIRLASELAEMVKRMCERGGSWGLEHHHHHH

SEQ ID NO: 37 - protein, 275 aa, Artificial Sequence
  Synthetic
GTREELAKELLRSLREQAESLARQLRLQREGSSDEDVKELAAEQIKLIMEQLLLIAELTLGRSEAAELALDAIRQALEACRTMDNQEACTRLLKLAIQMLELATRAPDAEAAKLALEAAKKAIELANRHPGSQAAEDATKLAQQAMEAVRLALKLYEEHPNADIADLCRRAAAEAAEAASKAAELAQRHPDSQAARDAIKLASQAAEAVKLACELAQEHPNADKAKLCILLASAAALLASIAAMLAQRHPDSQEARDMIRIASELAELVKEICER

SEQ ID NO: 38 - protein, 276 aa, Artificial Sequence
  Synthetic
  MISC_FEATURE (1)..(1): optionally absent
MGTREELAKELLRSLREQAESLARQLRLQREGSSDEDVKELAAEQIKLIMEQLLLIAELTLGRSEAAELALDAIRQALEACRTMDNQEACTRLLKLAIQMLELATRAPDAEAAKLALEAAKKAIELANRHPGSQAAEDATKLAQQAMEAVRLALKLYEEHPNADIADLCRRAAAEAAEAASKAAELAQRHPDSQAARDAIKLASQAAEAVKLACELAQEHPNADKAKLCILLASAAALLASIAAMLAQRHPDSQEARDMIRIASELAELVKEICER

SEQ ID NO: 39 - protein, 233 aa, Artificial Sequence
  Synthetic
GDEEKKKELLKQLEDSLIELIRILAELKEMLERLEKNPDKDTIVKVLKVIVKAIEASVANQAISAMNQGADANAKDSDGRTPLHHAAEAGAAAVVKVAIDAGADVNEKDSDGRTPLHHAAENGHAEVVTLLIEKGADVNEKDSDGRTPLHHAAENGHDEVVLILLLKGADVNAKDSDGRTPLHHAAENGHKRVVLVLILAGADVNTSDSDGRTPLDLAREHGNEEVVKALEKQ

SEQ ID NO: 40 - protein, 245 aa, Artificial Sequence
  Synthetic
  MISC_FEATURE (1)..(1): optionally absent
  MISC_FEATURE (235)..(245): optionally absent
MGDEEKKKELLKQLEDSLIELIRILAELKEMLERLEKNPDKDTIVKVLKVIVKAIEASVANQAISAMNQGADANAKDSDGRTPLHHAAEAGAAAVVKVAIDAGADVNEKDSDGRTPLHHAAENGHAEVVTLLIEKGADVNEKDSDGRTPLHHAAENGHDEVVLILLLKGADVNAKDSDGRTPLHHAAENGHKRVVLVLILAGADVNTSDSDGRTPLDLAREHGNEEVVKALEKQGGWLEHHHHHH

SEQ ID NO: 41 - protein, 287 aa, Artificial Sequence
  Synthetic
  MISC_FEATURE (245)..(245): X is S or C
  MISC_FEATURE (287)..(287): X is S or C
GGSELEIVIRLQILNLELARKLLEAVARLQELNIDLVRKTSELTDEKTIREEIRKVKEESKRIVKEAEDEIKKAALISADLAAKAIKRAIDRAKKLLEKGEKEDAEDVLREARSAIRLVTELLERIAKNSSTPEEALRAAELLVRLIILLIKIAALLAAAGNKEEADKVLDEAKELIERVRELLEKISKNSDTPELSKRAKELELILRLADLAIKAMKNTGSDEARQAVKEMARLAKEALEMGMXEAAKAAIELLELLAEAFAGSDVASLAVKAIAKIAETALRNGX

SEQ ID NO: 42 - protein, 288 aa, Artificial Sequence
  Synthetic
  MISC_FEATURE (1)..(1): optionally absent
  MISC_FEATURE (246)..(246): X is S or C
  MISC_FEATURE (288)..(288): X is S or C
MGGSELEIVIRLQILNLELARKLLEAVARLQELNIDLVRKTSELTDEKTIREEIRKVKEESKRIVKEAEDEIKKAALISADLAAKAIKRAIDRAKKLLEKGEKEDAEDVLREARSAIRLVTELLERIAKNSSTPEEALRAAELLVRLIILLIKIAALLAAAGNKEEADKVLDEAKELIERVRELLEKISKNSDTPELSKRAKELELILRLADLAIKAMKNTGSDEARQAVKEMARLAKEALEMGMXEAAKAAIELLELLAEAFAGSDVASLAVKAIAKIAETALRNGX

SEQ ID NO: 43 - protein, 201 aa, Artificial Sequence
  Synthetic
  MISC_FEATURE (2)..(2): X is S or C
  MISC_FEATURE (44)..(44): X is S or C
GXDTAKEAIQRLEDLARKYSGSDVASLAVKAIEKIARTAVENGXEETAEEAEKRLRELAEDYQGSNVASLAASAIAEIAAARARFAAREMGDPRVEEIAKELERLAKEAAERVERRPDSEEDYRKLELAALIIKLFVSLLKQKRLAERLKELLRELERLQREGSSDEDVRELLREIKELVEEIEKLARKQEYLVTELAKMM

SEQ ID NO: 44 - protein, 222 aa, Artificial Sequence
  Synthetic
  MISC_FEATURE (1)..(1): optionally absent
  MISC_FEATURE (3)..(3): X is S or C
  MISC_FEATURE (45)..(45): X is S or C
  MISC_FEATURE (203)..(222): optionally absent
MGXDTAKEAIQRLEDLARKYSGSDVASLAVKAIEKIARTAVENGXEETAEEAEKRLRELAEDYQGSNVASLAASAIAEIAAARARFAAREMGDPRVEEIAKELERLAKEAAERVERRPDSEEDYRKLELAALIIKLFVSLLKQKRLAERLKELLRELERLQREGSSDEDVRELLREIKELVEEIEKLARKQEYLVTELAKMMGGSGGSGGSGGSLEHHHHHH

SEQ ID NO: 45 - protein, 272 aa, Artificial Sequence
  Synthetic
GKELEIVARLQQLNIELARKLLEAVARLQELNIDLVRKTSELTDEKTIREEIRKVKEESKRIVEEAEQEIRKAEAESLRLTAEAAADAARKAALRMGDERVRRLAAELVRLAQEAAEEATRDPNSSDQNEALRLIILAIEAAVRALDKAIEKGDPEDRERAREMVRAAVRAAELVQRYPSASAANEALKALVAAIDEGDKDAARCAEELVEQAEEALRKKNPEEARAVYEAARDVLEALQRLEEAKRRGDEEERREAEERLRQACERARKKN

SEQ ID NO: 46 - protein, 284 aa, Artificial Sequence
  Synthetic
  MISC_FEATURE (1)..(1): optionally absent
  MISC_FEATURE (274)..(284): optionally absent
MGKELEIVARLQQLNIELARKLLEAVARLQELNIDLVRKTSELTDEKTIREEIRKVKEESKRIVEEAEQEIRKAEAESLRLTAEAAADAARKAALRMGDERVRRLAAELVRLAQEAAEEATRDPNSSDQNEALRLIILAIEAAVRALDKAIEKGDPEDRERAREMVRAAVRAAELVQRYPSASAANEALKALVAAIDEGDKDAARCAEELVEQAEEALRKKNPEEARAVYEAARDVLEALQRLEEAKRRGDEEERREAEERLRQACERARKKNGGSLEHHHHHH